(19) United States
(12) Patent Application Publication
(10) Pub. No.: US 2006/0217390 A1
(43) Pub. Date: Sep. 28, 2006

(54) Cycloalkl, aryl and heteroaryl amino isothiazoles for the treatment of Hepatitis C virus

(76) Inventors: Esmir Gunic, Irvine, CA (US); Todd Appleby, Laguna Niguel, CA (US); Weidong Zhong, Laguna Niguel, CA (US); Shunqi Yan, Irvine, CA (US); Ching Ha (Viel) Lai, Cambrige, MA (US)

Correspondence Address: BROWN, RAYSMAN, MILLSTEIN, FELDER & STEINER LLP, 900 THIRD AVENUE, NEW YORK, NY, 10022, US

(21) Appl. No.: 11/361,581

(22) Filed: Feb. 24, 2006

Related U.S. Application Data
(60) Provisional application No. 60/655,746, filed on Feb. 24, 2005.
(60) Provisional application No. 60/668,936, filed on Apr. 6, 2005.
(60) Provisional application No. 60/669,547, filed on Apr. 8, 2005.
(60) Provisional application No. 60/669,791, filed on Apr. 8, 2005.
(60) Provisional application No. 60/669,792, filed on Apr. 8, 2005.

(51) Int. Cl. A61K 31/506; A61K 31/501; A61K 31/497; A61K 31/4545; A61K 31/4439; C07D 417/14

(52) U.S. Cl. 514/252.05; 514/255.05; 514/256; 514/318; 514/340; 544/238; 544/333; 544/405; 546/193; 546/271.1

(57) ABSTRACT

This invention concerns certain 5-(substituted amino) isothiazoles, compounds of Formula I, and salts thereof, 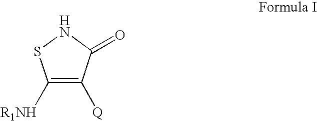
where Q is CN, NHCONRₐR_{b}, or CONRₐR_{b}, where Rₐ and R_{b} are defined herein; and R₁ is cyclohexyl, adamantan-1-yl, indan-1-yl, phenyl, benzyl, pyridyl, pyridylmethyl, or pyrimidyl, where all aromatic R₁ groups are optionally substituted, provided that when R₁ is phenyl, then R₁ bears at least one non-alkyl substituent, and further provided that R₁ is not 4-chloro-3-trichloromethyl-phenyl; The invention also concerns the tautomeric 5-(substituted amino)-isothiazol-3(2H)-ones, and the use of such compounds to treat Hepatitis C infection. It also concerns thiocarbamoyl acetamides, which are synthetic precursors to the isothiazoles.

### CROSS-REFERENCES TO RELATED APPLICATIONS

This application claims the benefit of U.S. Provisional Application No. 60/655,746, filed Feb. 24, 2005; U.S. Provisional Application No. 60/668,936, filed Apr. 6, 2005; U.S. Provisional Application No. 60/669,547, filed Apr. 8, 2005; U.S. Provisional Application No. 60/669,791, filed Apr. 8, 2005; and U.S. Provisional Application No. 60/669,792, filed Apr. 8, 2005. All of these applications are hereby incorporated herein by reference.

### BACKGROUND OF THE INVENTION

### Field of the Invention

This invention concerns certain 5-(substituted amino) isothiazoles, as well as the tautomeric 5-(substituted amino)-isothiazol-3(2H)-ones, and the use of such compounds to treat Hepatitis C infection. It also concerns thiocarbamoyl acetamides which are synthetic precursors to the isothiazoles.

Hepatitis C virus (HCV) infection presents a significant worldwide health problem that affects approximately 170 million people, with about 30,000 new cases in the United States each year. HCV is not easily cleared by the host's immunological defenses, and as many as 85% of the people infected with HCV become chronically infected, often resulting in chronic liver disease (Hoofnagle, J. H. 1997, Hepatology 26: 15S-20S). A substantial portion of these infected individuals will slowly progress into severe liver diseases, including cirrhosis, liver failure and hepatocellular carcinoma (Lauer, G. M.; Walker B. D. 2001, N. Engl. J. Med. 345: 41-52). In addition, liver failure due to HCV is the leading cause of liver transplantation in the United States and Europe (Seeff, L. B. 2002, Hepatology 36(Suppl 1): 35S-46S; Adam R., et al. 2000, Lancet 356:621-627). The Centers for Disease Control and Prevention estimate that chronic hepatitis C virus infection is responsible for approximately 10,000 to 12,000 deaths in the United States annually. This number is expected to triple in the next 10 to 20 years without effective intervention.

The development of effective vaccines for prophylaxis and treatment of HCV infection has been unsuccessful due to various virus-specific difficulties, especially immune evasion. Nonetheless, treatment of chronic hepatitis C has achieved significant advances in recent years. The current standard therapy for chronic hepatitis C consists of a combination of pegylated interferon-alpha (IFNα) and ribavirin. It confers an overall sustained viral response (SVR) of around 54-56% among treated patients, but is less efficacious against infection by HCV genotype 1. The limited effectiveness and adverse side effects of the current therapy underscore the urgent need for development of more effective and HCV-specific antiviral therapeutics.

HCV is a single-stranded, positive-sense RNA virus belonging to the hepacivirus genus of the Flaviviridae family (Choo, Q. L. et al., 1989, Science 244:359-364). The 9.6 kb genome of HCV encodes a single polyprotein which is cleaved co- and post-translationally by cellular and viral proteases into at least four structural (C, E1, E2, and p7) and six non-structural (NS2, NS3, NS4A, NS4B, NS5A and NS5B) proteins. One of these nonstructural proteins is NS5B, the RNA-dependent RNA polymerase, which plays a central role in viral RNA replication and is therefore an attractive target for the development of antiviral intervention.

Similar to the replication of most positive-strand RNA viruses, it is believed that HCV forms membrane-associated replication complexes in catalyzing RNA synthesis during viral RNA replication. These replication complexes contain viral non-structural proteins (NS3, NS4A, NS4B, NS5A and NS5B), viral RNA and unidentified host cellular proteins. Recently, several groups have demonstrated the in vitro replication activity of HCV replicase complexes in a crude membrane fraction isolated from the HCV subgenomic replicon cells (Ali, N. et al. 2002, J. Virol. 76:12001-12007; Hardy, R. W. et al. 2003, J. Virol. 77:2029-2037; Lai, V. C. H. et al. 2003, J. Virol. 77:2295-2300). The successful replication of HCV RNA using authentic replicase complexes in vitro will facilitate molecular dissection of the replication process and provide a system to evaluate potential antiviral drugs against the entire replicase complex of HCV.

Substituted heterocyclic compounds have previously been described for use in various therapeutic applications. For example, selected 4-pyridimidinone derivatives have been described as immunomodulatory agents, and as active against tumors, inflammatory disease, certain viruses, parasites, and other pests (see e.g., WO 98/24782, U.S. Pat. No. 5,149,810, or EP No. 238059A3). In another example, selected substituted diamino-1,3,5 triazine derivatives were reported to exhibit HIV replication inhibiting properties (see, e.g., U.S. Pat. No. 6,380,194). In still other examples, use of various pyridine derivatives was reported as therapeutic for treatment of nitric oxide synthase (NOS)-mediated diseases, including adult respiratory distress syndrome, insulin-dependent diabetes mellitus, systemic lupus erythematosus, rejection after organ transplantation, asthma, pain, or ulcers, as described in U.S. Pat. No. 6,521,643.

However, none of the known heterocyclic derivatives have been shown to exhibit activity against RNA-dependent RNA polymerases, including the RNA polymerase NS5B of HCV. The absence of RNA-dependent RNA polymerases in mammals, and the fact that this enzyme appears to be essential to viral replication, suggest that the NS5B polymerase is an ideal target for anti-HCV therapeutics.

Thus, in view of the prevalence of HCV infection and the limited effectiveness and adverse side effects of the current therapies, there is a need for compositions and methods for effective treatment of viral infections, especially for effective treatment of HCV infections.

All references cited herein are hereby incorporated herein by reference.

### BRIEF SUMMARY OF THE INVENTION

This invention concerns certain 3-hydroxy-isothiazoles, substituted at the 4-position with a cyano, carbamoyl, or ureido group, and substituted at the 5-position with an arylamino, heteroarylamino, benzylamino, or heteroarylmethylamino group or with a cycloalkylamino group. These compounds have antiviral activity. This invention includes the tautomeric forms of these compounds, namely 4- and 5-substituted amino-isothiazol-3(2H)-ones, although all compounds here are depicted in the keto form. The invention also concerns certain substituted aryl- and heteroaryl-thiocarbamoyl-acetamides which are intermediates in the synthesis of the isothiazoles. This invention also includes all salts of these compounds.

Embodiments of this invention include compounds of Formula I, or a salt thereof, 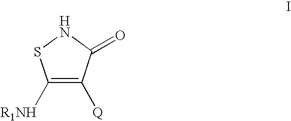
where all substituents are defined below in the detailed description; compounds of Formula IX, 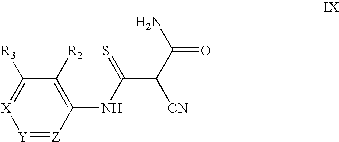
which are precursors of compounds of Formula I; and methods of treating Hepatitis C infection in a human comprising providing to a person in need of treatment thereof a therapeutically effective concentration of a compound of Formula I.

### DETAILED DESCRIPTION OF THE INVENTION

In one embodiment, the present invention provides a compound of Formula I or a salt thereof 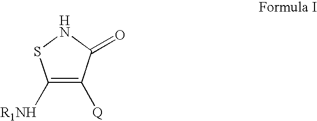
where Q is CN, NHCONRₐR_{b}, or CONRₐR_{b}, where Rₐ is C₁-C₃ alkyl or C₁-C₃ alkenyl, said alkyl and said alkenyl groups optionally substituted with, independently, 1, 2, or 3 halogen atoms, O—C₁₋₂ alkyl, C₁-C₃ alkenyl, and N(H)C₁₋₂ alkyl, R_{b} is H or C₁-C₃ alkyl; or Rₐ and R_{b}, together with the atoms to which they are attached, form a 5- or 6-membered ring, optionally containing one or two ring double bonds, and optionally containing one ring oxygen atom or one additional ring nitrogen atom; and R₁ is cyclohexyl, adamantan-1-yl, indan-1-yl, Ar¹ (CH₂)ₙ, or Ar², where n is zero or 1, Ar¹ is 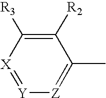
where X is C—R₄ or N, Y is C—R₅ or N, and Z is CH or N, provided that Ar contains no more than two ring nitrogen atoms; R₂, R₃, R₄, and R₅ are, independently, H, F, Cl, Br, I, OH, CN, CF₃, NO₂, C₁-C₆ alkyl, C₂-C₆ alkenyl, C₃-C₆ cycloalkyl, C₅-C₆ cycloalkenyl, O—C₁-C₆ alkyl, O—C₂-C₆ alkenyl, C₁-C₆ alkyl-C(═O)—, C₁-C₆ alkyl-O—C(═O)—, C₁-C₆ alkenyl-O—C(═O)—, C₁-C₆ alkyl-C(═O)O—, C₁-C₆ alkenyl-C(═O)O, isothiazolyl, isoxazolyl, isoxazolinyl, isoxazolidinyl, oxazolyl, oxazolinyl, oxazolidinyl, thiazolyl, thienyl, furyl, pyrrolyl, pyrrolinyl, pyrrolidinyl, thiazolinyl, thiazolidinyl, isothiazolinyl, isothiazolidinyl, imidazolinyl, imidazolyl, pyridyl, piperidinyl, phenyl, CH₃SO₂—, NH₂SO₂—, CH₃NHSO₂—, CH₃SO₂NH—, R₆R₇N—, R₆R₇NCH₂—, R₇C(═O)NH, or R₆R₇NC(═O), wherein R₆ and R₇ are, independently, H, C₁-C₄ alkyl, C₂-C₆ alkenyl; R₈—C≡C—, wherein R₈ is H, C₁-C₆ alkyl, C₂-C₆ alkenyl, C₃-C₆ cycloalkyl, C₅-C₆ cycloalkenyl, (CH₃)₂NCH₂—, (CH₃)₂NCH₂CH₂—, or phenyl; wherein all alkyl, cycloalkyl, alkenyl, and cycloalkenyl groups in Rₐ-R_{b} and R₂-R₈ are optionally substituted with one, two, or three halogen atoms, one C₁-C₃ alkyl group, or one hydroxyl group; and all aryl and heteroaryl groups in Rₐ-R_{b} and R₂—R₈ are optionally substituted with one hydroxy group and with one, two, or three groups selected from F, Cl, Br, I, and C₁-C₄ alkyl, provided that when n is zero and Ar is phenyl, then 1) R₂ is either H or halogen; and 2) at least one of R₂, R₃, R₄, and R₅ is neither H nor Cl; and further provided that Ar¹ is not 4-chloro-3-trichloromethyl-phenyl; and Ar² is 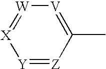
wherein V is N or CR₂, W is N or CR₃, X is N or CR₄, Y is N or CR₅, and Z is N or CH, provided that exactly two of V, W, X, Y, and Z are N, and further provided that the two ring nitrogen atoms are not adjacent, and R₂-R₅ are, independently, H, F, Cl, Br, I, OH, CN, CF₃, NO₂, C₁-C₆ alkyl, C₂-C₆ alkenyl, C₃-C₆ cycloalkyl, C₅-C₆ cycloalkenyl, C₅-C₆ cycloalkadienyl, O—C₁-C₆ alkyl, O—C₂-C₆ alkenyl, C₁-C₆ alkyl-C(═O)—, C₁-C₆ alkyl-O—C(═O)—, C₁-C₆ alkenyl-O—C(═O)—, C₁-C₆ alkyl-C(═O)O—, C₁-C₆ alkenyl-C(═O)O, isothiazolyl, isoxazolyl, isoxazolinyl, isoxazolidinyl, oxazolyl, oxazolinyl, oxazolidinyl, thiazolyl, thienyl, furyl, pyrrolyl, pyrrolinyl, pyrrolidinyl, thiazolinyl, thiazolidinyl, isothiazolinyl, isothiazolidinyl, imidazolinyl, imidazolyl, pyridyl, piperidinyl, phenyl, CH₃SO₂—, NH₂SO₂—, CH₃NHSO₂—, CH₃SO₂NH—, R₆R₇N—, R₆R₇NCH₂—, R₇C(═O)NH, or R₆R₇NC(═O), wherein R₆ and R₇ are, independently, H, C₁-C₄ alkyl, C₂-C₆ alkenyl; R₈—C≡C—, wherein R₈ is H, C₁-C₆ alkyl, C₂-C₆ alkenyl, C₃-C₆ cycloalkyl, C₅-C₆ cycloalkenyl, (CH₃)₂NCH₂—, (CH₃)₂NCH₂CH₂—, or phenyl; wherein all alkyl, cycloalkyl, alkenyl, and cycloalkenyl groups in R₂-R₅ and R₆-R₈ are optionally substituted with one, two, or three halogen atoms, one C₁-C₃ alkyl group, or one hydroxyl group; and all aryl and heteroaryl groups in R₂-R₅ and R₆-R₈ are optionally substituted with one, two, or three groups selected from F, Cl, Br, I, and C₁-C₄ alkyl;
Rₐ is H, C₁-C₃ alkyl or C₁-C₃ alkenyl, optionally substituted with O—C₁₋₂ alkyl, C₁-C₃ alkenyl, or N(H)C₁₋₂ alkyl; and R_{b} is C₁-C₃ alkyl, or Rₐ and R_{b}, together with the atoms to which they are attached, form a 5- or 6-membered ring, optionally containing one or two ring double bonds, and optionally containing one ring oxygen atom or one additional ring nitrogen atom.

In one subgeneric embodiment, the invention provides a compound of Formula Ia or a salt thereof.

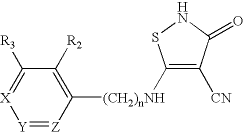

In one subgeneric embodiment, the invention provides a compound according to Formula Ia, in which n is zero, X is C—R₄, Y is C—R₅, and Z is CH.

In another embodiment the invention provides a compound according to Formula Ia, in which n is zero, X is C—R₄, Y is C—R₅, Z is CH and R₃ is CF₃, CH₃ or OCH₃.

In another embodiment the invention provides a compound according to Formula Ia, in which n is zero, X is C—R₄, Y is C—R₅, Z is CH, and R₄ is CF₃, CH₃ or OCH₃.

In another embodiment the invention provides a compound according to Formula Ia, in which n is zero, X is C—R₄, Y is C—R₅, Z is CH, and one of R₃ and R₄ is R₆—C≡C—.

In another embodiment the invention provides a compound according to Formula Ia, in which n is zero, X is C—R₄, Y is C—R₅, Z is CH, and one of R₃ and R₄ is CH₃C(═O)O— or CH₃C(═O)—.

In another embodiment the invention provides a compound according to Formula Ia, or a salt thereof, in which n is zero, X is C—R₄, Y is C—R₅, Z is CH, and one of R₃ and R₄ is furyl, pyrrolyl, pyrrolinyl, pyrrolidinyl, thiazolinyl, thiazolidinyl, isothiazolinyl, isothiazolidinyl, imidazolinyl, or imidazolyl.

In another embodiment the invention provides a compound according to Formula Ia, or a salt thereof, in which n is zero, X is C—R₄, Y is C—R₅, Z is CH, and one of R₃ and R₄ is pyridyl, piperidinyl, or phenyl.

In another embodiment the invention provides a compound according to Formula Ia, or a salt thereof, in which n is zero, X is C—R₄, Y is C—R₅, Z is CH, and one of R₃ and R₄ is C₃-C₆ cycloalkyl, mono-halo-substituted C₃-C₆ cycloalkyl, or C₅-C₆ cycloalkenyl.

In another embodiment the invention provides a compound according to Formula Ia, or a salt thereof, in which n is zero, X is C—R₄, Y is C—R₅, Z is CH, and one of R₃ and R₄ is cyclopropyl or methylcyclopropyl.

In another embodiment the invention provides a compound according to Formula Ia, or a salt thereof, in which n is zero, X is C—R₄, Y is C—R₅, Z is CH and one of R₃ and R₄ is OCH═CH₂, OCH₂CH₃, or OCH₂CH₂Cl.

In another embodiment the invention provides a compound according to Formula Ia, or a salt thereof, in which n is zero, X is C—R₄, Y is C—R₅, Z is CH, and one of R₃ and R₄ is halogen.

In another embodiment the invention provides a compound according to Formula Ia, or a salt thereof, in which n is zero, X is C—R₄, Y is C—R₅, Z is CH, and one of R₃ and R₄ is nitro or cyano.

In another embodiment the invention provides a compound according to Formula Ia, or a salt thereof, in which n is zero, X is C—R₄, Y is C—R₅, Z is CH, and one of R₃ and R₄ is R₆R₇N—, R₆R₇NCH₂—, R₇C(═O)NH, or R₆R₇NC(═O).

In another embodiment the invention provides a compound according to Formula Ia, or a salt thereof, in which n is zero, X is C—R₄, Y is C—R₅, Z is CH, and one of R₃ and R₄ is CH₃SO₂—, NH₂SO₂—, CH₃NHSO₂—, CH₃SO₂NH—.

In another embodiment the invention provides a compound according to Formula Ia, or a salt thereof, in which n is zero, X is C—R₄, Y is C—R₅, Z is CH, where none of R₃, R₄, or R₅ is H.

In another embodiment the invention provides a compound according to Formula Ia, or a salt thereof, in which n is zero, X is C—R₄, Y is C—R₅, Z is CH, where neither R₃ nor R₅ is H.

In another embodiment the invention provides a compound according to Formula Ia, or a salt thereof, in which n is zero, X is C—R₄, Y is C—R₅, Z is CH, and R₃ and R₅ are both halogen, both methyl, or both trifluoromethyl.

In another embodiment the invention provides a compound according to Formula Ia, or a salt thereof, in which n is zero, X is C—R₄, Y is C—R₅, Z is CH, and R₃ and R₅ are, independently, halo, C₁-C₄ alkoxy, cyclopropyl, C₂-C₄ alkenyl, or C₁-C₄ alkyl, said alkyl groups and the alkyl moieties of said alkoxy groups optionally substituted with 1-3 halogen atoms.

In another embodiment the invention provides a compound according to Formula Ia, or a salt thereof, in which n is zero, X is C—R₄, Y is C—R₅, Z is CH, and R₃ and R₅ are both methyl, both chloro or both trifluoromethyl.

In another embodiment the invention provides a compound according to Formula Ia, or a salt thereof, in which n is zero, X is C—H, Y is C—R₅, Z is CH, and R₃ and R₅ are both methyl, both chloro, or both trifluoromethyl.

In another embodiment the invention provides a compound according to Formula Ia, or a salt thereof, in which n is zero, X is C—R₄, Y is C—R₅, Z is CH, and R₃ and R₄ are both methyl, both chloro, or both trifluoromethyl.

In another subgeneric embodiment the invention provides a compound according to Formula Ia, or a salt thereof, in which n is 1.

In another subgeneric embodiment the invention provides a compound according to Formula Ia, or a salt thereof, in which n is 1; R₂ is H, halogen, CF₃, or CH₃; X is C—R₄; Y is C—R₅; Z is CH; and R₃ is CF₃, CH₃ or OCH₃.

In another embodiment the invention provides a compound according to Formula Ia, or a salt thereof, in which n is 1; R₂ is H, halogen, CF₃, or CH₃; X is C—R₄; Y is C—R₅; Z is CH; and R₄ is CF₃, CH₃ or OCH₃.

In another embodiment the invention provides a compound according to Formula Ia, or a salt thereof, in which n is 1; R₂ is H, halogen, CF₃, or CH₃; X is C—R₄; Y is C—R₅; Z is CH; and one of R₃ and R₄ is R₆—C≡C—.

In another embodiment the invention provides a compound according to Formula Ia, or a salt thereof, in which n is 1; R₂ is H, halogen, CF₃, or CH₃; X is C—R₄; Y is C—R₅; Z is CH; and one of R₃ and R₄ is CH₃C(═O)O— or CH₃C(═O)—.

In another embodiment the invention provides a compound according to Formula Ia, or a salt thereof, in which n is 1; R₂ is H, halogen, CF₃, or CH₃; X is C—R₄; Y is C—R₅; Z is CH; and one of R₃ and R₄ is furyl, pyrrolyl, pyrrolinyl, pyrrolidinyl, thiazolinyl, thiazolidinyl, isothiazolinyl, isothiazolidinyl, imidazolinyl, or imidazolyl.

In another embodiment the invention provides a compound according to Formula Ia, or a salt thereof, in which n is 1; R₂ is H, halogen, CF₃, or CH₃; X is C—R₄; Y is C—R₅; Z is CH; and one of R₃ and R₄ is pyridyl, piperidinyl, or phenyl.

In another embodiment the invention provides a compound according to Formula Ia, or a salt thereof, in which n is 1; R₂ is H, halogen, CF₃, or CH₃; X is C—R₄; Y is C—R₅; Z is CH; and one of R₃ and R₄ is C₃-C₆ cycloalkyl, mono-halo-substituted C₃-C₆ cycloalkyl, or C₅-C₆ cycloalkenyl.

In another embodiment the invention provides a compound according to Formula Ia, or a salt thereof, in which n is 1; R₂ is H, halogen, CF₃, or CH₃; X is C—R₄; Y is C—R₅; Z is CH; and one of R₃ and R₄ is cyclopropyl or methylcyclopropyl.

In another embodiment the invention provides a compound according to Formula Ia, or a salt thereof, in which n is 1; R₂ is H, halogen, CF₃, or CH₃; X is C—R₄; Y is C—R₅; Z is CH; and one of R₃ and R₄ is OCH═CH₂, OCH₂CH₃, or OCH₂CH₂Cl.

In another embodiment the invention provides a compound according to Formula Ia, or a salt thereof, in which n is 1; R₂ is H, halogen, CF₃, or CH₃; X is C—R₄; Y is C—R₅; Z is CH; and one of R₃ and R₄ is halogen.

In another embodiment the invention provides a compound according to Formula Ia, or a salt thereof, in which n is 1; R₂ is H, halogen, CF₃, or CH₃; X is C—R₄; Y is C—R₅; Z is CH; and one of R₃ and R₄ is nitro or cyano.

In another embodiment the invention provides a compound according to Formula Ia, or a salt thereof, in which n is 1; R₂ is H, halogen, CF₃, or CH₃; X is C—R₄; Y is C—R₅; Z is CH; and one of R₃ and R₄ is R₇N—, R₆R₇NCH₂—, R₇C(═O)NH, or R₆R₇NC(═O).

In another embodiment the invention provides a compound according to Formula Ia, or a salt thereof, in which n is 1; R₂ is H, halogen, CF₃, or CH₃; X is C—R₄; Y is C—R₅; Z is CH; and one of R₃ and R₄ is CH₃SO₂—, NH₂SO₂—, CH₃NHSO₂—, CH₃SO₂NH—.

In another embodiment the invention provides a compound according to Formula Ia, or a salt thereof, in which n is 1; R₂ is H, halogen, CF₃, or CH₃; X is C—R₄; Y is C—R₅; and Z is CH; where none of R₃, R₄, or R₅ is H.

In another embodiment the invention provides a compound according to Formula Ia, or a salt thereof, in which n is 1; R₂ is H, halogen, CF₃, or CH₃; X is C—R₄; Y is C—R₅; and Z is CH; where neither R₃ nor R₅ is H.

In another embodiment the invention provides a compound according to Formula Ia, or a salt thereof, in which n is 1, X is C—R₄, Y is C—R₅, Z is CH, where none of R₃, R₄, or R₅ is H.

In another embodiment the invention provides a compound according to Formula Ia, or a salt thereof, in which n is 1, X is C—R₄, Y is C—R₅, Z is CH, where neither R₃ nor R₄ is H.

In another embodiment the invention provides a compound according to Formula Ia, or a salt thereof, in which n is 1, X is C—R₄, Y is C—R₅, Z is CH, and R₃ and R₅ are both halogen.

In another embodiment the invention provides a compound according to Formula Ia, or a salt thereof, in which n is 1, X is C—R₄, Y is C—R₅, Z is CH, and R₃ and R₅ are both C₁-C₃ alkyl, each such C₁-C₃ alkyl optionally substituted with 1-3 halogen atoms.

In another embodiment the invention provides a compound according to Formula Ia, or a salt thereof, in which n is 1, X is C—R₄, Y is C—R₅, Z is CH, and R₃ and R₅ are either both methyl, both chloro, or both trifluoromethyl.

In another embodiment the invention provides a compound according to Formula Ia, or a salt thereof, in which n is 1, X is C—H, Y is C—R₅, Z is CH, and R₃ and R₅ are either both chloro or both trifluoromethyl.

In another embodiment the invention provides a compound according to Formula Ia, or a salt thereof, in which n is 1, X is C—R₄, Y is C—R₅, Z is CH, and R₃ and R₄ are either both chloro or both trifluoromethyl.

In another embodiment, the invention provides a compound according to Formula Ia, or a salt thereof, in which n is zero, and one of X, Y, and Z is N.

In one subgeneric embodiment, the invention provides a compound according to Formula Ia, or a salt thereof, in which n is 1, and one of X, Y, and Z is N.

In another embodiment, the invention provides a compound according to Formula Ia, or a salt thereof, in which n is zero, and one of X, Y, and Z is N.

In one subgeneric embodiment, the invention provides a compound according to Formula Ia, or a salt thereof, in which n is 1, and one of X, Y, and Z is N.

In another embodiment, the invention provides a compound according to Formula Ia, or a salt thereof, in which n is zero, X is N, and R₂, R₃ and R₅ are, independently, H, halogen, methyl, or trifluoromethyl.

In another embodiment, the invention provides a compound according to Formula Ia, or a salt thereof, in which n is zero, X is N, and two of R₂, R₃, and R₅ are H.

In another embodiment, the invention provides a compound according to Formula Ia, or a salt thereof, in which n is zero, X is N, R₃ is C₁-C₆ alkyl, C₂-C₆ alkenyl, C₃-C₆ cycloalkyl, C₅-C₆ cycloalkenyl, C₅-C₆ cycloalkadienyl, and R₂ and R₅ are H.

In another embodiment, the invention provides a compound according to Formula Ia, or a salt thereof, in which n is zero, X is N, R₃ is C₁-C₆ alkyl-C(═O)—, C₁-C₆ alkyl-O—C(═O)—, C₁-C₆ alkenyl-O—C(═O)—, C₁-C₆ alkyl-C(═O)O—, C₁-C₆ alkenyl-C(═O)O, and R₂ and R₅ are H.

In another embodiment, the invention provides a compound according to Formula Ia, or a salt thereof, in which n is zero, X is N, R₃ is C₁-C₄ alkyl, or C₁-C₄ alkoxy, each optionally substituted with 1-3 halogen atoms, and R₂ and R₅ are H.

In another embodiment, the invention provides a compound according to Formula Ia, or a salt thereof, in which n is zero, Y is N, R₂ or R₃ is C₁-C₄ alkyl, or C₁-C₄ alkoxy, each optionally substituted with 1-3 halogen atoms, and remaining R's are H.

In another embodiment, the invention provides a compound according to Formula Ia, or a salt thereof, in which n is zero, Y is N, R₄ is halogen or cyano, and R₂ and R₃ are H.

In another embodiment, the invention provides a compound according to Formula Ia, or a salt thereof, in which n is zero, Y is N, and two of R₂, R₃, and R₄ are H.

In another embodiment, the invention provides a compound according to Formula Ia, or a salt thereof, in which n is zero, Y is N, and R₂, R₃, and R₄ are, independently, H, halogen, methyl, or trifluoromethyl.

In another embodiment, the invention provides a compound according to Formula Ia, or a salt thereof, in which n is zero, Z is N, and R₂, R₃, R₄, and R₅ are, independently, H, halogen, methyl, or trifluoromethyl.

In another embodiment, the invention provides a compound according to Formula Ia, or a salt thereof, in which n is zero, Z is N, and three of R₂, R₃, R₄, and R₅ are H.

In another embodiment, the invention provides a compound according to Formula Ia, or a salt thereof, in which n is zero, Z is N, and two of R₂, R₃, R₄, and R₅ are H.

In another embodiment, the invention provides a compound according to Formula Ia, or a salt thereof, in which n is zero, Z is N, and one of R₂, R₃, R₄, and R₅ is halo, nitro, hydroxy, or alkoxy.

In another embodiment, the invention provides a compound according to Formula Ia, or a salt thereof, in which n is zero, Z is N, and one of R₂, R₃, R₄, and R₅ is CH₃SO₂—, NH₂SO₂—, CH₃NHSO₂—, CH₃SO₂NH—, R₆R₇N—, R₆R₇NCH₂—, R₇C(═O)NH, or R₆R₇NC(═O).

In another embodiment, the invention provides a compound according to Formula Ia, or a salt thereof, in which n is zero, Z is N, and one of R₂, R₃, R₄, and R₅ is oxazolyl, oxazolinyl, oxazolidinyl, thiazolyl, thienyl, furyl, pyrrolyl, pyrrolinyl, pyrrolidinyl, thiazolinyl, thiazolidinyl, or isothiazolinyl.

In another embodiment, the invention provides a compound according to Formula Ia, or a salt thereof, in which n is zero, Z is N, and one of R₂, R₃, R₄, and R₅ is C₃-C₆ cycloalkyl, C₅-C₆ cycloalkenyl, or C₅-C₆ cycloalkadienyl, optionally substituted as described above.

In another embodiment the invention provides a compound according to Formula Ia, or a salt thereof, in which n is zero, X is C—R₄, Y is C—R₅, Z is CH, and one of R₃ and R₄ is CH₃SO₂—, NH₂SO₂—, CH₃NHSO₂—, CH₃SO₂NH—.

In another embodiment the invention provides a compound according to Formula Ia, or a salt thereof, in which n is zero, X is C—R₄, Y is C—R₅, Z is CH, where none of R₃, R₄, or R₅ is H.

In another embodiment the invention provides a compound according to Formula Ia, or a salt thereof, in which n is zero, X is C—R₄, Y is C—R₅, Z is CH, where neither R₃ nor R₅ is H.

In another embodiment the invention provides a compound according to Formula Ia, or a salt thereof, in which n is zero, X is C—R₄, Y is C—R₅, Z is CH, and R₃ and R₅ are both halogen.

In another embodiment the invention provides a compound according to Formula Ia, or a salt thereof, in which n is zero, X is C—R₄, Y is C—R₅, Z is CH, and R₃ and R₅ are both C₁-C₃ alkyl, each such C₁-C₃ alkyl optionally substituted with 1-3 halogen atoms.

In another embodiment the invention provides a compound according to Formula Ia, or a salt thereof, in which n is zero, X is C—R₄, Y is C—R₅, Z is CH, and R₃ and R₅ are either both chloro or both trifluoromethyl.

In another embodiment the invention provides a compound according to Formula Ia, or a salt thereof, in which n is zero, X is C—H, Y is C—R₅, Z is CH, and R₃ and R₅ are either both chloro or both trifluoromethyl.

In another subgeneric embodiment, the invention provides a compound according to Formula Ia, or a salt thereof, in which n is zero or 1 and X is N.

In another subgeneric embodiment, the invention provides a compound according to Formula Ia, or a salt thereof, in which n is zero or 1 and Y is N.

In another subgeneric embodiment, the invention provides a compound according to Formula Ia, or a salt thereof, in which n is zero or 1 and Z is N.

In another embodiment, the invention provides a compound according to Formula Ia, or a salt thereof, in which n is zero or 1, X is N, and R₂, R₃ and R₅ are, independently, H, halogen, C₁-C₆ alkyl, C₂-C₆ alkenyl, C₂-C₆ alkynyl, or C₃-C₆ cycloalkyl, all optionally substituted as described above.

In another embodiment, the invention provides a compound according to Formula Ia, or a salt thereof, in which n is zero or 1, Y is N, and R₂, R₃ and R₄ are, independently, H, halogen, C₁-C₆ alkyl, C₂-C₆ alkenyl, C₂-C₆ alkynyl, or C₃-C₆ cycloalkyl, all optionally substituted as described above.

In another embodiment, the invention provides a compound according to Formula Ia, or a salt thereof, in which n is zero or 1, Z is N, and R₂, R₃, R₄, and R₅ are, independently, H, halogen, C₁-C₆ alkyl, C₂-C₆ alkenyl, C₂-C₆ alkynyl, or C₃-C₆ cycloalkyl, all optionally substituted as described above.

In another embodiment, the invention provides a compound according to Formula Ia, or a salt thereof, in which n is zero or 1, X is N, and R₂, R₃ and R₅ are, independently, H, halogen, methyl, methoxy, nitro, cyano, CH₃C≡C—, Ph-C≡C—, or trifluoromethyl.

In another embodiment, the invention provides a compound according to Formula Ia, or a salt thereof, in which n is zero or 1, Y is N, and R₂, R₃ and R₄ are, independently, H, halogen, methyl, methoxy, nitro, cyano, CH₃C≡C—, Ph-C≡C—, or trifluoromethyl.

In another embodiment, the invention provides a compound according to Formula Ia, or a salt thereof, in which n is zero or 1, Z is N, and R₂, R₃, R₄, and R₅ are, independently, H, halogen, methyl, methoxy, nitro, cyano, CH₃C≡C—, Ph-C≡C—, or trifluoromethyl.

In another embodiment the invention provides a compound according to Formula Ia, or a salt thereof, in which n is zero or 1, X is N, Y is C—R₅, Z is CH, and R₃ and R₄ are either both chloro or both trifluoromethyl.

In another embodiment, the invention provides a compound according to Formula Ia, or a salt thereof, in which n is zero or 1, X is N, and two of R₂, R₃, and R₅ are H.

In another embodiment, the invention provides a compound according to Formula Ia, or a salt thereof, in which n is zero or 1, Y is N, and two of R₂, R₃, and R₄ are H.

In another embodiment, the invention provides a compound according to Formula Ia, or a salt thereof, in which n is zero or 1, Y is N, and R₂, R₃, and R₄ are, independently, H, halogen, methyl, or trifluoromethyl.

In another embodiment, the invention provides a compound according to Formula Ia, or a salt thereof, in which n is zero or 1, Z is N, and R₂, R₃, R₄, and R₅ are, independently, H, halogen, methyl, or trifluoromethyl.

In another embodiment, the invention provides a compound according to Formula Ia, or a salt thereof, in which n is zero or 1, Z is N, and three of R₂, R₃, R₄, and R₅ are H.

In another embodiment, the invention provides a compound according to Formula Ia, or a salt thereof, in which n is zero or 1, Z is N, and two of R₂, R₃, R₄, and R₅ are H.

In another embodiment the invention provides a compound according to Formula Ia, or a salt thereof, in which n is zero or 1, X is CR₄, Y is C—R₅, Z is N, and R₃ and R₄ are, independently, methyl, methoxy, fluoro, chloro, bromo, iodo, acetyl, acetamido, amino sulfonyl, or methylamino sulfonyl.

In another embodiment the invention provides a compound according to Formula Ia, or a salt thereof, in which n is zero or 1, X is CH, Y is C—R₅, Z is N, and R₃ and R₅ are, independently, methyl, methoxy, fluoro, chloro, bromo, iodo, acetyl, acetamido, amino sulfonyl, or methylamino sulfonyl.

In another embodiment the invention provides a compound according to Formula Ia, or a salt thereof, in which n is zero or 1, X is CR₄, Y is C—R₅, Z is N, R₄ is methyl, methoxy, hydroxy, halo, or cyano, and R₃ and R₅ are, independently, methyl, methoxy, fluoro, chloro, bromo, iodo, acetyl, acetamido, or methylamino sulfonyl.

In another embodiment the invention provides a compound according to Formula Ia, or a salt thereof, in which n is zero or 1, X is CH, Y is C—R₅, Z is N, and R₃ and R₄ are either both chloro or both trifluoromethyl.

In another embodiment, the invention provides a compound according to Formula Ia, or a salt thereof, in which n is zero or 1, X is N, R₂ is H, and one of R₃ and R₅ is a monocyclic heteroaryl group, optionally substituted as described above.

In another embodiment, the invention provides a compound according to Formula Ia, or a salt thereof, in which n is zero or 1, Y is N, R₂ is H, and one of R₃ and R₄ is a monocyclic heteroaryl group, optionally substituted as described above.

In another embodiment, the invention provides a compound according to Formula Ia, or a salt thereof, in which n is zero or 1, Z is N, R₂ is H, and one of R₃, R₄, and R₅ is a monocyclic heteroaryl group, optionally substituted

In another embodiment, the invention provides a compound according to Formula Ia, or a salt thereof, in which n is zero, X is N, R₂ is H, and one of R₃ and R₅ is a monocyclic heteroaryl group, optionally substituted with methyl, trifluoromethyl, or halogen.

In another embodiment, the invention provides a compound according to Formula Ia, or a salt thereof, in which n is zero, Y is N, R₂ is H, and one of R₃ and R₄ is a monocyclic heteroaryl group, optionally substituted with methyl, trifluoromethyl, or halogen.

In another embodiment, the invention provides a compound according to Formula Ia, or a salt thereof, in which n is zero, Z is N, R₂ is H, and one of R₃, R₄, and R₅ is a monocyclic heteroaryl group, optionally substituted with methyl, trifluoromethyl, or halogen.

In another embodiment, this invention provides a compound of Formula II or a salt thereof, which is a compound of Formula I in which n=zero or 1, and R₁ is cyclohexyl, adamantan-1-yl, indan-1-yl.

In another embodiment, the invention provides a compound of Formula III or a salt thereof 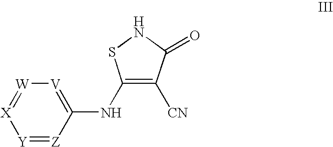
where all variables and substituents are defined as for formula I.

In one subgeneric embodiment this invention provides a compound of Formula IIIa or a salt thereof, 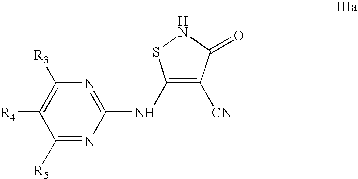
where R₃, R₄, and R₅ are defined as for Formula I.
In another subgeneric embodiment, this invention provides a compound of Formula IIIb or a salt thereof 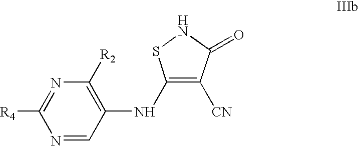
where R₂ and R₄ are defined as for Formula I.
In another subgeneric embodiment, this invention provides a compound of Formula IIIc or a salt thereof 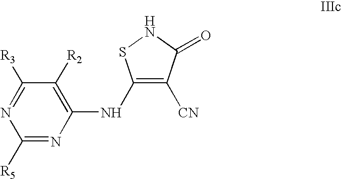
where R₂, R₃, and R₅ are defined as for Formula I.

In another embodiment, this invention provides a compound of formula IIIa or IIIb, or a salt thereof, where R₄ is C₁-C₄ alkyl, C₁-C₄ alkoxy, C₂-C₄ alkenyl, C₂-C₄ alkynyl, phenyl, or C₂-C₆ cycloalkyl, all optionally substituted as described above, or H, halo, cyano, nitro, acetyl, or amino sulfonyl.

In another embodiment, this invention provides a compound of formula IIIa or IIIc, or a salt thereof, where R₃ is C₁-C₄ alkyl, C₁-C₄ alkoxy, C₂-C₄ alkenyl, C₂-C₄ alkynyl, phenyl, or C₂-C₆ cycloalkyl, all optionally substituted as described above, or H, halo, cyano, nitro, acetyl, or amino sulfonyl.

In another embodiment, this invention provides a compound of formula IIIa or IIIc, or a salt thereof, where R₅ is C₁-C₄ alkyl, C₁-C₄ alkoxy, C₂-C₄ alkenyl, C₂-C₄ alkynyl, phenyl, or C₂-C₆ cycloalkyl, all optionally substituted as described above, or H, halo, cyano, nitro, acetyl, or amino sulfonyl.

In another embodiment, this invention provides a compound of formula IIIa, or a salt thereof, where R₄ is H, CF₃, or methyl, and R₃ and R₅ are, independently, C₁-C₄ alkyl, C₁-C₄ alkoxy, C₂-C₄ alkenyl, C₂-C₄ alkynyl, phenyl, or C₂-C₆ cycloalkyl, all optionally substituted as described above, or H, halo, cyano, nitro, acetyl, or amino sulfonyl.

In another embodiment, this invention provides a compound of formula IIIa, or a salt thereof, where R₄ is H, CF₃, or methyl, and R₃ and R₅ are, independently, H, halo, cyano, nitro, acetyl, acetamido, CH₃C(═O), CH₃C(═O)O, C₂-C₆ cycloalkyl, all alkyl groups optionally substituted as described above.

In another embodiment, this invention provides a compound of Formula I, or a salt thereof, wherein is zero and Q is NHCONRₐR_{b}.

In another embodiment, this invention provides a compound of Formula IVa or IVb or a salt thereof

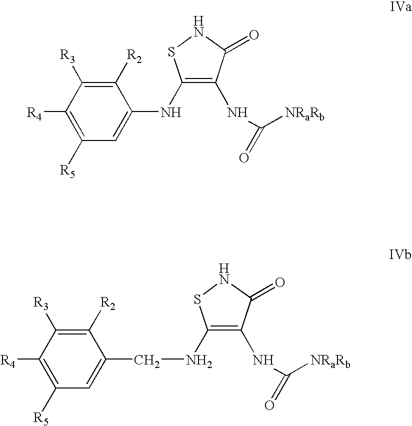

In another embodiment, this invention provides a compound of formula IVa or IVb, in which NRₐR_{b} is NH(CH₃) or N(CH₃)₂.

In another embodiment, this invention provides a compound of formula IVa or IVb or a salt thereof, in which NRₐR_{b} is a 5- or 6-membered ring, containing zero, one, or two ring double bonds.

In a more specific embodiment, this invention provides a compound of formula IVa or IVb or a salt thereof, in which NRₐR_{b} is morpholyl, isoxazolyl, oxazolyl, oxazolinyl, oxazolidinyl, piperidyl, pyrrolyl, pyrrolinyl, or pyrrolidinyl.

In another embodiment the invention provides a compound according to Formula Iva or IVb or a salt thereof, in which R₂ and R₅ are, independently, H, halogen, CF₃, or CH₃; and one of R₃ and R₄ is R₆R₇N—, R₆R₇NCH₂—, R₇C(═O)NH, or R₆R₇NC(═O).

In another embodiment the invention provides a compound according to Formula IVa or IVb or a salt thereof, in which R₂ and R₅ are, independently, H, halogen, CF₃, or CH₃; and one of R₃ and R₄ is CH₃SO₂—, NH₂SO₂—, CH₃NHSO₂—, CH₃SO₂NH—.

In another embodiment the invention provides a compound according to Formula IVa or IVb or a salt thereof, in which R₂ and R₅ are, independently, H, halogen, CF₃, or CH₃; and where neither of R₃ and R₄ is H.

In another embodiment the invention provides a compound according to Formula IVa or IVb or a salt thereof, in which R₂ is H, halogen, CF₃, or CH₃; and where neither R₄ nor R₅ is H.

In another embodiment, this invention provides a compound of formula IVa or IVb or a salt thereof, in which NRₐR_{b} is NH(CH₃) or N(CH₃)₂, where none of R₃, R₄, or R₅ is H.

In another embodiment, this invention provides a compound of formula IVa or IVb or a salt thereof, in which NRₐR_{b} is NH(CH₃) or N(CH₃)₂, R₂ and R₄ are H, and R₃ and R₅ are, independently, halogen, methyl, methoxy, trifluoromethyl, isopropyl, cyclopropyl, HC≡C—, CH₃C≡C—, or nitro.

In another embodiment, this invention provides a compound of formula IVa or IVb or a salt thereof, in which NRₐR_{b} is NHCH₃ or N(CH₃)₂; and R₄ is C₁-C₄ alkyl, HC≡C—, or C₃-C₆ cycloalkyl.

In another embodiment, this invention provides a compound of formula IVa or IVb or a salt thereof, in which R₄ is pyrrolyl, pyrrolidinyl, morpholyl, piperidinyl, piperazinyl, thiazolidinyl, oxazolidinyl, or imidazolinyl.

In another embodiment, this invention provides a compound of formula IVa or IVb or a salt thereof, in which NRₐR_{b} is NHEt or NEt₂.

In another embodiment, this invention provides a compound of formula IVa or IVb or a salt thereof, in which NRₐR_{b} is NH(C₁-C₃)alkyl or NH(C₁-C₃)alkenyl, wherein the alkyl or alkenyl group is optionally substituted with methoxy or ethoxy.

In another more specific embodiment, this invention provides a compound of formula IVa or IVb or a salt thereof, in which R₂ and R₃ are both H, and in which R₄ is other than H.

In another more specific embodiment, this invention provides a compound of IVa or IVb or a salt thereof, in which R₂ and R₃ are both H, and in which R₅ is other than H.

In another more specific embodiment, this invention provides a compound of formula IVa or IVb or a salt thereof, in which R₂ and R₃ are both H, in which and R₅ is other than H.

In another more specific embodiment, this invention provides a compound of formula IVa or IVb or a salt thereof, in which NRₐR_{b} is NHCH₃, and R₄ is thiazol-2-yl, thien-2-yl, 2-furyl, pyrrol-2-yl, oxazol-2-yl, isothiazol-3-yl, isoxazol-3-yl, phenyl, imidazol-2-yl, thiazol-4-yl, thien-3-yl, 3-furyl, pyrrol-3-yl, oxazol-4-yl, 3-isothiazol-3-yl, isoxazol-4-yl, 4-methylphenyl, or imidazol-4-yl.

In another more specific embodiment, this invention provides a compound of formula IVa or IVb or a salt thereof, in which NRₐR_{b} is NHCH₃, and R₃ or R₄ is CH₃C≡C—, phenyl-C≡C—, CH₃CH(OH)C≡C—, CH₃CH₂C≡C—, cyclopropyl-C≡C—, 1-hydroxy-cyclopentyl-C≡C—, 2-hydroxy-cyclopentyl-C≡C—, 3-hydroxy-cyclopentyl-C≡C—, cyclopentyl-C≡C—, isopropyl-C≡C—, tert-butyl-C≡C—, cyclohexyl-C≡C—, cyclohexen-1-yl-C≡C—, or (CH₃)₂NCH₂—C≡C—.

In another more specific embodiment, this invention provides a compound of formula IVa or IVb or a salt thereof, in which NRₐR_{b} is NHCH₃, and R₃ or R₄ is thiazol-2-yl, thien-2-yl, 2-furyl, pyrrol-2-yl, oxazol-2-yl, isothiazol-3-yl, isoxazol-3-yl, phenyl, imidazol-2-yl, thiazol-4-yl, thien-3-yl, 3-furyl, pyrrol-3-yl, oxazol-4-yl, 3-isothiazol-3-yl, isoxazol-4-yl, 4-methylphenyl, or imidazol-4-yl.

In another more specific embodiment, this invention provides a compound of formula IVA OR IVB or a salt thereof, in which NRₐR_{b} is NHCH₃, and R₃ or R₄ is CH₃C≡C—, phenyl-C≡C—, CH₃CH(OH)C≡C—, CH₃CH₂C≡C—, cyclopropyl-C≡C—, 1-hydroxy-cyclopentyl-C≡C—, 2-hydroxy-cyclopentyl-C—C—, 3-hydroxy-cyclopentyl-C≡C—, cyclopentyl-C≡C—, isopropyl-C≡C—, tert-butyl-C—C—, cyclohexyl-C≡C—, cyclohexen-1-yl-C≡C—, or (CH₃)₂NCH₂—C≡C—.

In another more specific embodiment, this invention contemplates a compound of formula IVa or IVb or a salt thereof, in which NRₐR_{b} is pyrrolidinyl; R₂ and R₃ are both H; and R₄ is ethoxy.

In another more specific embodiment, this invention contemplates a compound of formula IVa or IVb or a salt thereof, in which NR₄R₅ is pyrrolidinyl; R₂ and R₃ are both H; and R₄ is chloromethyl.

In another more specific embodiment, this invention contemplates a compound of formula IVa or IVb or a salt thereof, in which NRₐR_{b} is pyrrolidinyl; R₂ and R₃ are both H; and R₄ or R₅ is amino, dimethylamino, or methylamino.

In another more specific embodiment, this invention contemplates a compound of formula IVa or IVb or a salt thereof, in which NRₐR_{b} is pyrrolidinyl; R₂ and R₃ are both H; and R₄ or R₅ is cyclopentadien-5-yl, acetyl, vinyl, t-butyl, acetoxy, acetamide, methylaminocarbonyl, methanesulfonylamino, or methanesulfonyl.

In another more specific embodiment, this invention contemplates a compound of formula IVa or IVb or a salt thereof, in which NRₐR_{b} is NHCH₃; R₂ and R₃ are both H; and R₄ is selected from n-propanoyl, chloroacetyl, acetoxy, iso-propylcarbonyl, iso-propylcarbonyloxy, 2-chloro-n-propanoyl, 2-chloro-iso-propoxy, (CH₂Cl)₂—CH—, (CH₂Cl)₂—CHO—, 2-methyl- or 2-chloro-cyclopentyl; and 2-methyl- or 2-chloro-cyclohexyl.

In another more specific embodiment, this invention contemplates a compound of formula IVa or IVb or a salt thereof, in which NRₐR_{b} is NHCH₃; R₂ and R₃ are both H; and R₄ or R₅ is methanesulfonyl, methanesulfonylamino, or cyclopentadien-5-yl.

In another more specific embodiment, this invention contemplates a compound of formula IVa or IVb or a salt thereof, in which NRₐ is NHCH₃; R₂ and R₃ are both H; and R₄ is propen-2-yl, 2-hydroxyethyl, ethoxy, chloromethyl, amino, methylamino, dimethylamino, or dimethylaminomethyl.

In a further more specific embodiment, this invention contemplates a compound of formula IVa or IVb or a salt thereof, in which R₅ is CH₃; R₂ and R₃ are both H; and R₄ is acetyl.

In a further more specific embodiment, this invention contemplates a compound of formula IVa or IVb or a salt thereof, in which R₅ is CH₃; R₂ and R₃ are both H; and R₄ is vinyl.

In another more specific embodiment, this invention contemplates a compound of formula IVa or IVb or a salt thereof, which R₄ is propyn-1-yl.

In another more specific embodiment, this invention contemplates a compound of formula IVa or IVb or a salt thereof, in which NRₐR_{b} is pyrrolidinyl; R₂ and R₃ are both H; and R₅ is dimethylamino.

In another more specific embodiment, this invention contemplates a compound of formula IVa or IVb or a salt thereof, in which NRₐR_{b} is pyrrolidinyl; R₂ and R₃ are both H; and R₅ is cyclopentadien-5-yl.

In another more specific embodiment, this invention contemplates a compound of formula IVa or IVb or a salt thereof, in which NRₐR_{b} is pyrrolidinyl; R₂ and R₃ are both H; and R₅ is acetyl or acetoxy.

In another more specific embodiment, this invention contemplates a compound of formula IVa or IVb or a salt thereof, in which NRₐR_{b} is pyrrolidinyl; R₂ and R₃ are both H; and R₅ is vinyl, t-butyl, acetoxy, acetamido, methanesulfonyl, methanesulfonylamino, or methylaminocarbonyl.

In another more specific embodiment, this invention contemplates a compound of formula IVa or IVb or a salt thereof, in which NR₄R₅ is pyrrolidinyl; R₂ and R₃ are both H; and R₅ is propen-2-yl, 2-hydroxyethyl, ethoxy, chloromethyl, amino, dimethylamino, or dimethylaminomethyl.

In another more specific embodiment, this invention contemplates a compound of formula IVa or IVb or a salt thereof, in which NRₐR_{b} is NHCH₃; R₂ and R₃ are both H; and R₅ is cyclopentadien-5-yl, acetyl, acetoxy, vinyl, acetamido, methylaminocarbonyl, or t-butyl.

In another more specific embodiment, this invention contemplates a compound of formula IVa or IVb or a salt thereof, in which NRₐR_{b} is NHCH₃ and R₅ is propyn-1-yl, cyclopentadien-5-yl, acetyl, or vinyl.

In another more specific embodiment, this invention contemplates a compound of formula IVa or IVb or a salt thereof, in which R₄ and R₅ are either both methyl or both ethyl; and in which R₂ and R₃ are both H.

In a more specific embodiment, this invention contemplates a compound of formula IVa or IVb or a salt thereof, in which R₄ and R₅ are either both halo or both trifluoromethyl; and R₂ and R₃ are both H.

In another more specific embodiment, this invention contemplates a compound of formula IVa or IVb or a salt thereof, in which R₃ and R₅ are the same and are halo, methyl, or trifluoromethyl; and R₂ and R₄ are both H.

In another more specific embodiment, this invention contemplates a compound of formula IVa or IVb or a salt thereof, in which R₄ and R₅ are both CH₃; R₂ and R₃ are both H; and R₄ is dimethylaminomethyl.

In another more specific embodiment, this invention contemplates a compound of formula IVa or IVb or a salt thereof, in which R₄ and R₅ are both CH₃; R₂ and R₃ are both H; and R₄ is propyn-1-yl.

In another more specific embodiment, this invention contemplates a compound of formula IVa or IVb or a salt thereof, in which NRₐR_{b} is pyrrolyl; R₃ and R₅ are both H; and either R₂ or R₄ is fluoromethyl.

In another more specific embodiment, this invention contemplates a compound of formula IVa or IVb or a salt thereof, in which NRₐR_{b} is pyrrolidinyl or NHCH₃; R₃ and R₄ are both H; and R₂ is methoxy, acetyl, methyl, fluoro, vinyl, or ethoxy.

In another more specific embodiment, this invention contemplates a compound of formula IVa or IVb or a salt thereof, in which NRₐR_{b} is NHCH₃ or dimethylamino; R₂ and R₃ are both H; R₅ is H, methyl, or halo; and R₄ is thiazol-2-yl, thien-2-yl, 2-furyl, pyrrol-2-yl, oxazol-2-yl, isothiazol-3-yl, isoxazol-3-yl, phenyl, imidazol-2-yl, thiazol-4-yl, thien-3-yl, 3-furyl, pyrrol-3-yl, oxazol-4-yl, 3-isothiazol-3-yl, isoxazol-3-yl, 4-methylphenyl, or imidazol-4-yl.

In another more specific embodiment, this invention contemplates a compound of formula IVa or IVb or a salt thereof, in which NRₐR_{b} is NHCH₃, R₂, R₃, and R₄ are H, and R₅ is thiazol-2-yl, thien-2-yl, 2-furyl, pyrrol-2-yl, oxazol-2-yl, isothiazol-3-yl, isoxazol-3-yl, phenyl, imidazol-2-yl, thiazol-4-yl, thien-3-yl, 3-furyl, pyrrol-3-yl, oxazol-4-yl, 3-isothiazol-3-yl, isoxazol-3-yl, 4-methylphenyl, or imidazol-4-yl.

In another embodiment, this invention provides a compound of Formula V, or a salt thereof, 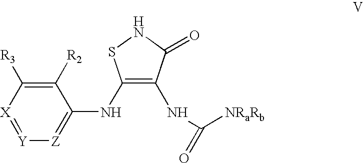
wherein one of X, Y, and Z is N.

In a more specific generic embodiment, the invention provides a compound of Formula V, or a salt thereof, wherein X is N.

In a more specific generic embodiment, the invention provides a compound of Formula V, or a salt thereof, wherein Y is N.

In a more specific generic embodiment, the invention provides a compound of Formula V, or a salt thereof, wherein Z is N.

In a more specific generic embodiment, this invention provides a compound of Formula V, or a salt thereof, in which NRₐR_{b} is NH(C₁-C₃)alkyl or NH(C₁-C₃)alkenyl, wherein the alkyl or alkenyl group is optionally substituted with methoxy or ethoxy.

In another more specific generic embodiment, the invention provides a compound of Formula V, or a salt thereof, wherein NRₐR_{b} is pyrrolyl, pyrrolidinyl, NHCH₃, or N(CH₃)₂.

In another embodiment, this invention provides a compound of formula V, or a salt thereof, in which NRₐR_{b} is NHEt or NEt₂.

In another embodiment, this invention provides a compound of formula V, or a salt thereof, in which NRₐR_{b} is piperidinyl, piperazinyl, or morpholinyl. in another subgeneric embodiment the invention provides a compound of Formula V, or a salt thereof, wherein one of R₂, R₃, or R₄ is thiazolyl, thienyl, furyl, pyrrolyl, oxazolyl, or imidazolyl.

In a more specific embodiment the invention provides a compound of Formula V, or a salt thereof, wherein X is N; R₂ and R₃ are both hydrogen; and R₅ is halogen.

In a more specific embodiment the invention provides a compound of Formula V, or a salt thereof, wherein X is N; R₂ and R₃ are both hydrogen; and R₅ is isothiazolyl, isoxazolyl, oxazolyl, oxazolinyl, oxazolidinyl, thiazolyl; thienyl; furyl; pyrrolyl; pyrrolinyl, pyrrolidinyl, thiazolinyl, thiazolidinyl, isothiazolinyl, isothiazolidinyl, imidazolinyl, imidazolyl; pyridyl; or phenyl.

In a more specific embodiment the invention provides a compound of Formula V, or a salt thereof, wherein Y is N; R₂ and R₃ are both hydrogen; and R₄ is isothiazolyl, isoxazolyl, oxazolyl, oxazolinyl, oxazolidinyl, thiazolyl; thienyl; furyl; pyrrolyl; pyrrolinyl, pyrrolidinyl, thiazolinyl, thiazolidinyl, isothiazolinyl, isothiazolidinyl, imidazolinyl, imidazolyl; pyridyl; or phenyl.

In another more specific embodiment, this invention contemplates a compound of Formula V, or a salt thereof, wherein X is N; R₃ is methylamino, dimethylamino, methylaminocarbonyl, ethylaminocarbonyl, dimethylaminocarbonyl, CH₃O(C═O), CH₃CH₂O(C═O), CH₃(C═O)O—, CH₃CH₂(C═O)O—, HOCH₂O(C═O), CH₂(OH)CH₂O(C═O), ClCH₂(C═O)O—, CH₃SO₂—, ClCH₂CH₂(C═O)O—, CH₃(C═O)NH—, CH₃CH₂(C═O)NH—, NH₂SO₂—; CH₃NHSO₂—; CH₃SO₂NH—, R₅ is methyl; and R₂ is H.

In another more specific embodiment, this invention contemplates a compound of Formula V, or a salt thereof, wherein X is N, R₂ is hydroxymethyl, 1-hydroxyethyl, 2-hydroxyethyl, propen-3-yl, 1-methylethenyl, or n-propyl; and R₃ and R₄ are both H.

In another more specific embodiment, this invention contemplates a compound of Formula V, or a salt thereof, wherein X is N; R₅ is hydroxymethyl, 1-hydroxyethyl, 2-hydroxyethyl, propen-3-yl, 1-methylethenyl, or n-propyl; R₃ is methyl; and R₂ is H.

In another, more specific embodiment, this invention contemplates a compound of Formula V, or a salt thereof, wherein X is N; R₅ is cyclopropyl, cyclobutyl, or cyclopentyl; and R₂ and R₃ are both H.

In another embodiment the invention contemplates a compound of Formula V, or a salt thereof, wherein Y is N; R₄ is thiazol-2-yl, thien-2-yl, thien-3-yl, 2-furyl, 3-furyl, pyrrol-2-yl, isothiazol-3-yl, isoxazol-3-yl, phenyl, pyrrol-3-yl, oxazol-2-yl, 4-oxazolin-3-yl, oxazolidin-5-yl, 4-methylphenyl, or imidazol-4-yl, or imidazol-2-yl; NRₐR_{b} is dimethylamino; and R₂ and R₃ are both H.

In another embodiment the invention contemplates a compound of Formula V, or a salt thereof, wherein X is N; R₃ is thiazol-2-yl, thien-2-yl, thien-3-yl, 2-furyl, 3-furyl, pyrrol-2-yl, isothiazol-3-yl, oxazol-2-yl, isoxazol-3-yl, phenyl, pyrrol-3-yl, 2-pyrrolin-3-yl, oxazol-4-yl, 4-methylphenyl, or imidazol-4-yl, or imidazol-2-yl, NRₐR_{b} is pyrrolyl; and R₂ and R₅ are both H.

In another, more specific embodiment, this invention contemplates a compound of Formula V, or a salt thereof, wherein Y is N; R₄ is cyclopropyl, cyclobutyl, cyclopentyl, cyclopent-2-en-yl, cyclopent-1-en-yl, cyclopent-3-en-yl, cyclohexyl; NRₐR_{b} is dimethylamino; and R₂ and R₃ are, independently, H or methyl.

In another, more specific embodiment, this invention contemplates a compound of Formula V, or a salt thereof, wherein Y is N; R₄ is 2-methyl- or 2-chloro-cyclopropyl; 2-methyl- or 2-chloro-cyclobutyl; 2-methyl- or 2-chloro-cyclopentyl; or 2-methyl- or 2-chloro-cyclohexyl; NRₐR_{b} is dimethylamino; and R₂ and R₃ are both H.

In another more specific embodiment, this invention contemplates a compound of Formula V, or a salt thereof, wherein Y is N; R₄ is hydroxymethyl, 1-hydroxyethyl, 2-hydroxyethyl, propen-3-yl, 1-methylethenyl, or n-propyl; NRₐR_{b} is dimethylamino; and R₂ and R₃ are both H.

In another, more specific embodiment, this invention contemplates a compound of Formula V, or a salt thereof, wherein Y is N; R₄ is acetyl, n-propanoyl, chloroacetyl, acetoxy, iso-propylcarbonyl, iso-propylcarbonyloxy, 2-chloro-n-propanoyl, 2-chloro-iso-propoxy, (CH₂Cl)₂—CH—, (CH₂Cl)₂—CHO—, 2-methyl- or 2-chloro-cyclopentyl; or 2-methyl- or 2-chloro-cyclohexyl; NRₐR_{b} is pyrrolyl; and R₂ and R₃ are both H.

In another, more specific embodiment, this invention contemplates a compound of Formula V, or a salt thereof, wherein Y is N; R₄ is acetyl, n-propanoyl, chloroacetyl, acetoxy, iso-propylcarbonyl, iso-propylcarbonyloxy, 2-chloro-n-propanoyl, 2-chloro-iso-propoxy, (CH₂Cl)₂—CH—, (CH₂Cl)₂—CHO—, 2-methyl- or 2-chloro-cyclopentyl; or 2-methyl- or 2-chloro-cyclohexyl; NRₐR_{b} is NHCH₃; and R₂ and R₃ are both H.

In another embodiment the invention contemplates a compound of Formula V, or a salt thereof, wherein Z is N, R₂ is thiazol-2-yl, thien-2-yl, 2-furyl, pyrrol-2-yl, oxazol-2-yl, isothiazol-3-yl, isoxazol-3-yl, phenyl, imidazol-2-yl, thiazol-4-yl, thien-3-yl, 3-furyl, pyrrol-3-yl, oxazol-4-yl, 3-isothiazol-3-yl, isoxazol-3-yl, 4-methylphenyl, or imidazol-4-yl; NRₐR_{b} is NHCH₃; and R₂ and R₃ are both H.

In another, more specific embodiment, the invention contemplates a compound of Formula V, or a salt thereof, wherein Y is N; R₄ is thiazol-2-yl, thien-2-yl, 2-furyl, pyrrol-2-yl, oxazol-2-yl, imidazol-2-yl, isothiazol-3-yl, isoxazol-3-yl, phenyl, imidazol-4-yl, thiazol-4-yl, thien-3-yl, 3-furyl, pyrrol-3-yl, oxazol-4-yl, isothiazol-4-yl, isoxazol-4-yl, 4-methylphenyl, or imidazol-4-yl; NRₐR_{b} is NHCH₃; and R₂ and R₃ are both H.

In another, more specific embodiment, the invention contemplates a compound of Formula V, or a salt thereof, wherein Z is N; R₄ is thiazol-2-yl, thien-2-yl, 2-furyl, pyrrol-2-yl, oxazol-2-yl, imidazol-2-yl, isothiazol-3-yl, isoxazol-3-yl, phenyl, imidazol-4-yl, thiazol-4-yl, thien-3-yl, 3-furyl, pyrrol-3-yl, oxazol-4-yl, isothiazol-4-yl, isoxazol-4-yl, 4-methylphenyl, or imidazol-4-yl; NRₐR_{b} is NHCH₃; and R₂ and R₃ are both H.

In another embodiment the invention contemplates a compound of Formula V, or a salt thereof; wherein Y is N; R₄ is CH₃C≡C—, phenyl-C≡C—, CH₃CH(OH)C≡C—, CH₃CH₂C≡C—, cyclopropyl-C≡C—, 1-hydroxy-cyclopentyl-C≡C—, 2-hydroxy-cyclopentyl-C≡C—, 3-hydroxy-cyclopentyl-C≡C—, cyclopentyl-C≡C—, isopropyl-C≡C—, tert-butyl-C≡C—, cyclohexyl-C≡C—, cyclohexen-1-yl-C≡C—, or (CH₃)₂NCH₂—C≡C—; NRₐR_{b} is NHCH₃; and R₂ and R₃ are both H.

In another, more specific embodiment, this invention contemplates a compound of Formula V, or a salt thereof, wherein Y is N; R₄ is cyclopropyl, cyclobutyl, cyclopentyl, cyclopent-2-en-yl, cyclopent-1-en-yl, cyclopent-3-en-yl, cyclohexyl; NRₐR_{b} is N(CH₃)₂; and R₂ and R₃ are both H.

In another, more specific embodiment, this invention contemplates a compound of Formula V, or a salt thereof, wherein Y is N; R₄ is 2-methyl-cyclopropyl or 2-chloro-cyclopropyl; 2-methyl- or 2-chloro-cyclobutyl; 2-methyl- or 2-chloro-cyclopentyl; or 2-methyl- or 2-chloro-cyclohexyl; NRₐR_{b} is N(CH₃)₂; and R₂ and R₃ are both H.

In another more specific embodiment, this invention contemplates a compound of Formula V, or a salt thereof, wherein Y is N; R₄ is hydroxymethyl, 1-hydroxyethyl, 2-hydroxyethyl, propen-3-yl, 1-methylethenyl, or n-propyl; NRₐR_{b} is N(CH₃)₂; and R₂ and R₃ are both H.

In another, more specific embodiment, this invention contemplates a compound of Formula V, or a salt thereof, wherein Y is N; R₄ is acetyl, n-propanoyl, chloroacetyl, acetoxy, iso-propylcarbonyl, iso-propylcarbonyloxy, 2-chloro-n-propanoyl, 2-chloro-iso-propoxy, (CH₂Cl)₂—CH—, (CH₂Cl)₂—CHO—, 2-methyl-cyclopentyl; 2-chloro-cyclopentyl; or 2-methyl-cyclohexyl or 2-chloro-cyclohexyl; NRₐR_{b} is pyrrolidinyl; and R₂ and R₃ are both H.

In another embodiment the invention contemplates a compound of Formula V, or a salt thereof, wherein Z is N; R₃ is thiazol-2-yl, thien-2-yl, 2-furyl, pyrrol-2-yl, oxazol-2-yl, isothiazol-3-yl, isoxazol-3-yl, phenyl, imidazol-2-yl, thiazol-4-yl, thien-3-yl, 3-furyl, pyrrol-3-yl, oxazol-4-yl, 3-isothiazol-3-yl, isoxazol-3-yl, 4-methylphenyl, or imidazol-4-yl; NR₄R₅ is pyrrolidinyl; and R₂ and R₄ are both H.

In another embodiment the invention contemplates a compound of Formula V, or a salt thereof, wherein Z is N; R₃ is thiazol-2-yl, thien-2-yl, 2-furyl, pyrrol-2-yl, oxazol-2-yl, isothiazol-3-yl, isoxazol-3-yl, phenyl, imidazol-2-yl, thiazol-4-yl, thien-3-yl, 3-furyl, pyrrol-3-yl, oxazol-4-yl, 3-isothiazol-3-yl, isoxazol-3-yl, 4-methylphenyl, or imidazol-4-yl; NRₐR_{b} is pyrrolidinyl; and R₂ and R₄ are both H.

In another, more specific embodiment, the invention contemplates a compound of Formula V, or a salt thereof, wherein X is N; R₅ is thiazol-2-yl, thien-2-yl, 2-furyl, pyrrol-2-yl, oxazol-2-yl, imidazol-2-yl, isothiazol-3-yl, isoxazol-3-yl, phenyl, imidazol-4-yl, thiazol-4-yl, thien-3-yl, 3-furyl, pyrrol-3-yl, oxazol-4-yl, isothiazol-4-yl, isoxazol-4-yl, 4-methylphenyl, or imidazol-4-yl; NRₐR_{b} is pyrrolidinyl; and R₂ and R₃ are both H.

In another, more specific embodiment, the invention contemplates a compound of Formula V, or a salt thereof, wherein Y is N; R₄ is thiazol-2-yl, thien-2-yl, 2-furyl, pyrrol-2-yl, oxazol-2-yl, imidazol-2-yl, isothiazol-3-yl, isoxazol-3-yl, phenyl, imidazol-4-yl, thiazol-4-yl, thien-3-yl, 3-furyl, pyrrol-3-yl, oxazol-4-yl, isothiazol-4-yl, isoxazol-4-yl, 4-methylphenyl, or imidazol-4-yl, NRₐR_{b} is pyrrolidinyl; and R₂ and R₃ are both H.

In another, more specific embodiment, the invention contemplates a compound of Formula V, or a salt thereof, wherein Z is N; R₄ is thiazol-2-yl, thien-2-yl, 2-furyl, pyrrol-2-yl, oxazol-2-yl, imidazol-2-yl, isothiazol-3-yl, isoxazol-3-yl, phenyl, imidazol-4-yl, thiazol-4-yl, thien-3-yl, 3-furyl, pyrrol-3-yl, oxazol-4-yl, isothiazol-4-yl, isoxazol-4-yl, 4-methylphenyl, or imidazol-4-yl; NRₐR_{b} is pyrrolidinyl; and R₂ and R₃ are both H.

In another embodiment the invention contemplates a compound of Formula V, or a salt thereof, wherein Y is N; R₄ is CH₃C≡C—, phenyl-C≡C—, CH₃CH(OH)C≡C—, CH₃CH₂C≡C—, cyclopropyl-C≡C—, 1-hydroxy-cyclopentyl-C≡C—, 2-hydroxy-cyclopentyl-C≡C—, 3-hydroxy-cyclopentyl-CE-C—, cyclopentyl-C≡C—, isopropyl-C≡C—, tert-butyl-C≡C—, cyclohexyl-C≡C—, cyclohexen-1-yl-C≡C—, or (CH₃)₂NCH₂—C≡C—; NRₐR_{b} is pyrrolidinyl; and R₂ and R₃ are both H.

In another embodiment, this invention contemplates a compound of Formula V, or a salt thereof, wherein X is N; R₃ is cyclopropyl, cyclobutyl, or cyclopentyl; NRₐR_{b} is pyrrolidinyl; and R₂ and R₅ are both H.

In another embodiment, this invention contemplates a compound of Formula V, or a salt thereof, wherein X is N, R₃ is 2-fluorocyclopropyl, 2-fluorocyclobutyl, 2-hydroxycyclopropyl, 2-hydroxycyclobutyl, 2-hydroxycyclopentyl, or 2-fluorocyclopentyl, NRₐR_{b} is pyrrolidinyl, and R₂ and R₅ are both H.

In another embodiment the invention contemplates a compound of Formula V, or a salt thereof, wherein Z is N; R₃ is thiazol-2-yl, thien-2-yl, 2-furyl, pyrrol-2-yl, oxazol-2-yl, isothiazol-3-yl, isoxazol-3-yl, phenyl, imidazol-2-yl, thiazol-4-yl, thien-3-yl, 3-furyl, pyrrol-3-yl, oxazol-4-yl, 3-isothiazol-3-yl, isoxazol-3-yl, 4-methylphenyl, or imidazol-4-yl; NR₄R₅ is pyrrolinyl; and R₂ and R₄ are both H.

In another embodiment the invention contemplates a compound of Formula V, or a salt thereof, wherein Z is N; R₃ is thiazol-2-yl, thien-2-yl, 2-furyl, pyrrol-2-yl, oxazol-2-yl, isothiazol-3-yl, isoxazol-3-yl, phenyl, imidazol-2-yl, thiazol-4-yl, thien-3-yl, 3-furyl, pyrrol-3-yl, oxazol-4-yl, 3-isothiazol-3-yl, isoxazol-3-yl, 4-methylphenyl, or imidazol-4-yl; NRₐR_{b} is pyrrolinyl; and R₂ and R₄ are both H.

In another, more specific embodiment, the invention contemplates a compound of Formula V, or a salt thereof, wherein X is N; R₂ is thiazol-2-yl, thien-2-yl, 2-furyl, pyrrol-2-yl, oxazol-2-yl, imidazol-2-yl, isothiazol-3-yl, isoxazol-3-yl, phenyl, imidazol-4-yl, thiazol-4-yl, thien-3-yl, 3-furyl, pyrrol-3-yl, oxazol-4-yl, isothiazol-4-yl, isoxazol-4-yl, 4-methylphenyl, or imidazol-4-yl; NRₐR_{b} is pyrrolinyl; and R₃ and R₅ are both H.

In another, more specific embodiment, the invention contemplates a compound of Formula V, or a salt thereof, wherein Y is N; R₄ is thiazol-2-yl, thien-2-yl, 2-furyl, pyrrol-2-yl, oxazol-2-yl, imidazol-2-yl, isothiazol-3-yl, isoxazol-3-yl, phenyl, imidazol-4-yl, thiazol-4-yl, thien-3-yl, 3-furyl, pyrrol-3-yl, oxazol-4-yl, isothiazol-4-yl, isoxazol-4-yl, 4-methylphenyl, or imidazol-4-yl, NRₐR_{b} is pyrrolinyl; and R₂ and R₃ are both H.

In another, more specific embodiment, the invention contemplates a compound of Formula V, or a salt thereof, wherein Z is N; R₄ is thiazol-2-yl, thien-2-yl, 2-furyl, pyrrol-2-yl, oxazol-2-yl, imidazol-2-yl, isothiazol-3-yl, isoxazol-3-yl, phenyl, imidazol-4-yl, thiazol-4-yl, thien-3-yl, 3-furyl, pyrrol-3-yl, oxazol-4-yl, isothiazol-4-yl, isoxazol-4-yl, 4-methylphenyl, or imidazol-4-yl; NRₐR_{b} is pyrrolinyl; and R₂ and R₃ are both H.

In another embodiment the invention contemplates a compound of Formula V, or a salt thereof, wherein Y is N; R₄ is CH₃C≡C—, phenyl-C≡C—, CH₃CH(OH)C≡C—, CH₃CH₂C≡C—, cyclopropyl-C≡C—, 1-hydroxy-cyclopentyl-C≡C—, 2-hydroxy-cyclopentyl-C≡C—, 3-hydroxy-cyclopentyl-C≡C—, cyclopentyl-C≡C—, isopropyl-C≡C—, tert-butyl-C≡C—, cyclohexyl-C≡C—, cyclohexen-1-yl-C≡C—, or (CH₃)₂NCH₂—C≡C—; NRₐR_{b} is pyrrolinyl; and R₂ and R₃ are both H.

In another embodiment, this invention contemplates a compound of Formula V, or a salt thereof, wherein X is N; R₃ is cyclopropyl, cyclobutyl, or cyclopentyl; NRₐR_{b} is pyrrolinyl; and R₂ and R₅ are both H.

In another embodiment, this invention contemplates a compound of Formula V, or a salt thereof, wherein X is N, R₃ is 2-fluorocyclopropyl, 2-fluorocyclobutyl, 2-hydroxycyclopropyl, 2-hydroxycyclobutyl, 2-hydroxycyclopentyl, or 2-fluorocyclopentyl, NRₐR_{b} is pyrrolinyl, and R₂ and R₅ are both H.

In another embodiment, this invention provides a compound of formula VI or a salt thereof.

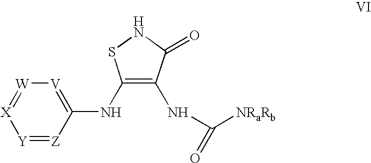

In one subgeneric embodiment, this invention provides a compound of Formula VIa, or a salt thereof.

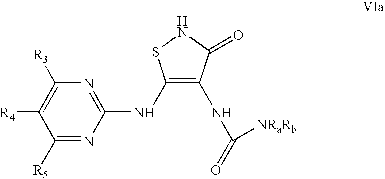

In another subgeneric embodiment, this invention provides a compound of Formula VIb, or a salt thereof.

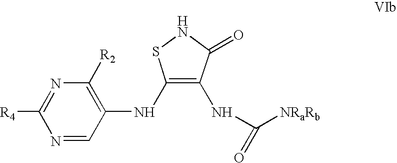

In one subgeneric embodiment, this invention provides a compound of Formula VIc, or a salt thereof. 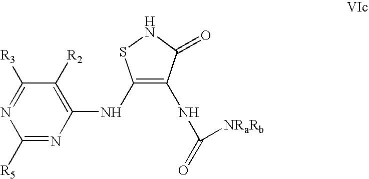
In another subgeneric embodiment, this invention provides a compound of any of Formulas VIa, VIb, or VIc, or a salt thereof, in which NRₐR_{b} is NHCH₃, N(CH₃)₂, pyrrolyl, pyrrolinyl, or pyrrolidinyl.

In another subgeneric embodiment, this invention provides a compound of either of Formulas VIa, or VIc, or a salt thereof, in which R₃ and R₅ are, independently, H, methyl, methoxy, halo, or trifluoromethyl.

In another subgeneric embodiment, this invention provides a compound Formula VIa, or a salt thereof, in which NRₐR_{b} is NHCH₃) or N(CH₃)₂; R₃ is acetyl, acetoxy; methyloxycarbonyl; hydroxymethyl, ethylaminocarbonyl, 2-chloroethyl, iso-propoxy, t-butyl, vinyl, propen-2-yl or cyclopenten-2-yl; and R₄ and R₅ are H.

In another subgeneric embodiment, this invention provides a compound Formula VIa, or a salt thereof, in which NRₐR_{b} is NHCH₃) or N(CH₃)₂; R₄ is F; Cl; Br; I; OH; CN; CF₃; NO₂; methyl, ethyl, n-propyl, iso-propyl, t-butyl, vinyl, propen-2-yl or cyclopropyl; and R₃ and R₅ are H.

In another subgeneric embodiment, this invention provides a compound Formula VIb, or a salt thereof, in which NRₐR_{b} is NHCH₃) or N(CH₃)₂; R₄ is acetyl, acetoxy; methyloxycarbonyl; hydroxymethyl, ethylaminocarbonyl, 2-chloroethyl, iso-propoxy, t-butyl, vinyl, propen-2-yl or cyclopenten-2-yl.

In another subgeneric embodiment, this invention provides a compound Formula VIb, or a salt thereof, in which NRₐR_{b} is NHCH₃) or N(CH₃)₂; R₄ is F; Cl; Br; I; OH; CN; CF₃; NO₂; methyl, ethyl, n-propyl, iso-propyl, t-butyl, vinyl, propen-2-yl or cyclopropyl; R₂ is H.

In another subgeneric embodiment, this invention provides a compound of any of Formulas VIb, or a salt thereof, in which NRₐR_{b} is NHCH₃ or N(CH₃)₂; R₄ is acetyl, acetoxy; methyloxycarbonyl; hydroxymethyl, ethylaminocarbonyl, 2-chloroethyl, iso-propoxy, t-butyl, vinyl, propen-2-yl or cyclopenten-2-yl; and R₂ is H.

In another subgeneric embodiment, this invention provides a compound of Formula VIc, or a salt thereof, in which NRₐR_{b} is NHCH₃ or N(CH₃)₂; and R₃, R₄, and R₅ are, independently, H, halo, cyclopropyl, methyl, methoxy, ethyl, n-propyl, iso-propyl, t-butyl, vinyl, or propen-2-yl.

In another subgeneric embodiment, this invention provides a compound of Formula VIc, or a salt thereof, in which NRₐR_{b} is NHCH₃ or N(CH₃)₂; R₄ is acetyl, acetoxy; methyloxycarbonyl; hydroxymethyl, ethylaminocarbonyl, 2-chloroethyl, iso-propoxy, t-butyl, vinyl, propen-2-yl or cyclopenten-2-yl; and R₃ and R₅ are H.

In another subgeneric embodiment, this invention provides a compound of Formula VIc, or a salt thereof, in which NRₐR_{b} is NHCH₃ or N(CH₃)₂; R₃ is F; Cl; Br; I; OH; CN; CF₃; NO₂; methyl, ethyl, n-propyl, iso-propyl, t-butyl, vinyl, propen-2-yl or cyclopropyl; and R₂ and R₅ are H.

In another subgeneric embodiment, this invention provides a compound of Formula VIc, or a salt thereof, in which NRₐR_{b} is NHCH₃ or N(CH₃)₂; R₃ is acetyl, acetoxy; methyloxycarbonyl; hydroxymethyl, ethylaminocarbonyl, 2-chloroethyl, iso-propoxy, t-butyl, vinyl, propen-2-yl or cyclopenten-2-yl; R₂ and R₅ are H.

In another subgeneric embodiment, this invention provides a compound of Formula VIc, or a salt thereof, in which NRₐR_{b} is pyrrolidinyl; R₃ is F; Cl; Br; I; OH; CN; CF₃; NO₂; methyl, ethyl, n-propyl, iso-propyl, t-butyl, vinyl, propen-2-yl or cyclopropyl; and R₄ and R₅ are H.

In another subgeneric embodiment, this invention provides a compound of Formula VIc, or a salt thereof, in which NRₐR_{b} is pyrrolidinyl; R₃ is acetyl, acetoxy, methyloxycarbonyl, hydroxymethyl, ethylaminocarbonyl, 2-chloroethyl, iso-propoxy, t-butyl, vinyl, propen-2-yl or cyclopenten-2-yl; and R₄ and R₅ are H.

In another embodiment, this invention provides a compound of Formula VII or a salt thereof,

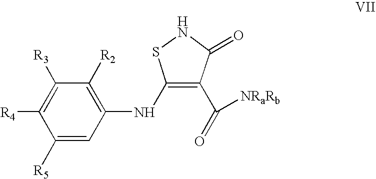

In one subgeneric embodiment, the invention provides a compound of Formula VII or a salt thereof, in which NRₐR_{b} is a 5- or 6-membered ring, containing zero, one, or two ring double bonds.

In a more specific subgeneric embodiment, this invention provides a compound of formula VII in which NRₐR_{b} is morpholyl, isoxazolyl, oxazolyl, oxazolinyl, oxazolidinyl, piperidyl, pyrrolyl, pyrrolinyl, or pyrrolidinyl.

In another subgeneric embodiment, this invention provides a compound of formula VII in which NRₐR_{b} is NHCH₃ or N(CH₃)₂.

In one subgeneric embodiment, the invention provides a compound of Formula VII or a salt thereof, in which NRₐR_{b} is NHCH₃ or N(CH₃)₂ and three of R₂-R₅ are hydrogen.

In one subgeneric embodiment, the invention provides a compound of Formula VII or a salt thereof, in which NRₐR_{b} is NHCH₃ or N(CH₃)₂ and two of R₂-R₅ are hydrogen.

In one subgeneric embodiment, the invention provides a compound of Formula VII or a salt thereof, in which NRₐR_{b} is NHCH₃ or N(CH₃)₂, R₅ is H, and one of R₂-R₄ is C₁-C₄ alkyl, HC≡C—, or C₃-C₆ cycloalkyl, all optionally substituted as described for Formula I.

In one subgeneric embodiment, the invention provides a compound of Formula VII or a salt thereof, in which NRₐR_{b} is NHCH₃ or N(CH₃)₂; R₅ is H; and one of R₂-R₄ is pyrrolyl, pyrrolidinyl, morpholyl, piperidinyl, piperazinyl, thiazolidinyl, oxazolidinyl, or imidazolinyl.

In one subgeneric embodiment, the invention provides a compound of Formula VII or a salt thereof, in which NRₐR_{b} is NHCH₃ or N(CH₃)₂; R₂, R₃ and R₅ are H; and R₄ is thiazol-2-yl, thien-2-yl, 2-furyl, pyrrol-2-yl, oxazol-2-yl, isothiazol-3-yl, isoxazol-3-yl, phenyl, imidazol-2-yl, thiazol-4-yl, thien-3-yl, 3-furyl, pyrrol-3-yl, oxazol-4-yl, 3-isothiazol-3-yl, isoxazol-4-yl, 4-methylphenyl, or imidazol-4-yl.

In one subgeneric embodiment, the invention provides a compound of Formula VII or a salt thereof, in which NRₐR_{b} is NHCH₃ or N(CH₃)₂; R₅ is H; and R₄ is CH₃C≡C—, phenyl-C≡C—, CH₃CH(OH)C≡C—, CH₃CH₂C≡C—, cyclopropyl-C≡C—, 1-hydroxy-cyclopentyl-C≡C—, 2-hydroxy-cyclopentyl-C≡C—, 3-hydroxy-cyclopentyl-C≡C—, cyclopentyl-C≡C—, isopropyl-C≡C—, tert-butyl-C—C—, cyclohexyl-C≡C—, cyclohexen-1-yl-C≡C—, or (CH₃)₂NCH₂—C≡C—.

In one subgeneric embodiment, the invention provides a compound of Formula VII or a salt thereof, in which NRₐR_{b} is NHCH₃ or N(CH₃)₂; R₂, R₃ and R₄ are H, and R₅ is thiazol-2-yl, thien-2-yl, 2-furyl, pyrrol-2-yl, oxazol-2-yl, isothiazol-3-yl, isoxazol-3-yl, phenyl, imidazol-2-yl, thiazol-4-yl, thien-3-yl, 3-furyl, pyrrol-3-yl, oxazol-4-yl, 3-isothiazol-3-yl, isoxazol-4-yl, 4-methylphenyl, or imidazol-4-yl.

In one subgeneric embodiment, the invention provides a compound of Formula VII or a salt thereof, in which NRₐR_{b} is NHCH₃ or N(CH₃)₂; R₂, R₃ and R₄ are H, and R₅ is CH₃C≡C—, phenyl-C—C—, CH₃CH(OH)C≡C—, CH₃CH₂C≡C—, cyclopropyl-C≡C—, 1-hydroxy-cyclopentyl-C≡C—, 2-hydroxy-cyclopentyl-C≡C—, 3-hydroxy-cyclopentyl-C≡C—, cyclopentyl-C≡C—, isopropyl-C≡C—, tert-butyl-C≡C—, cyclohexyl-C≡C—, cyclohexen-1-yl-C≡C—, or (CH₃)₂NCH₂—C≡C—.

In one subgeneric embodiment, the invention provides a compound of Formula VII or a salt thereof, in which NRₐR_{b} is NHCH₃ or N(CH₃)₂; R₂ and R₄ are H; and R₃ and R₅ are, independently, C₁-C₄ alkyl, C₁-C₄ alkoxy, C₂-C₄ alkenyl, all optionally substituted as described for Formula I, or halo.

In another embodiment, the invention provides a compound of Formula VIII or a salt thereof

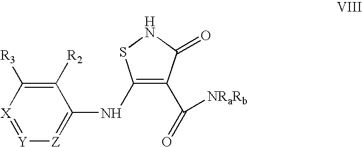

In one subgeneric embodiment, the invention provides a compound of Formula VIIIa or a salt thereof,

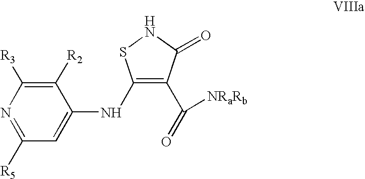

In one subgeneric embodiment, the invention provides a compound of Formula VIIIb or a salt thereof,

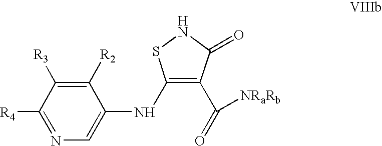

In one subgeneric embodiment, the invention provides a compound of Formula VIIIc or a salt thereof,

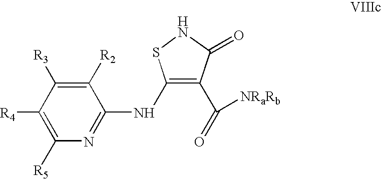

In one subgeneric embodiment, the invention provides a compound of Formula VIIIa, VIIIb, or VIIIc, or a salt thereof, in which NRₐR_{b} is NHCH₃ or N(CH₃)₂; and R₂ and R₃ are H.

In one subgeneric embodiment, the invention provides a compound of Formula VIIIa, or a salt thereof, in which NRₐR_{b} is NHCH₃ or N(CH₃)₂; R₂ is H; and R₃ and R₅ are, independently, F, Cl, CH₃, CF₃, and OCH₃.

In one subgeneric embodiment, the invention provides a compound of Formula VIIIa, VIIIb, or VIIIc, or a salt thereof, in which NRₐR_{b} is NHCH₃ or N(CH₃)₂; R₂ is H, and R₃ and R₅ are, independently, H, F, Cl, CH₃, CF₃, vinyl, and cyclopropyl.

In one subgeneric embodiment, the invention provides a compound of Formula VIIIa, VIIIb, or VIIIc, or a salt thereof, in which NRₐR_{b} is NHCH₃ or N(CH₃)₂; R₅ is H, and R₂ and R₃ are, independently, H, halo, or C₁-C₄ alkyl.

In one subgeneric embodiment, the invention provides a compound of Formula VIIIa, VIIIb, or VIIIc, or a salt thereof, in which NRₐR_{b} is NHCH₃ or N(CH₃)₂; R₂ is H; and R₃ and R₄ are, independently, H, halo, C₁-C₆ alkyl, C₁-C₆ alkoxy, C₂-C₆ alkenyl, C₃-C₆ cycloalkyl, acetamide, acetyl, acetoxy, 2-hydroxyethyl, methylamino, dimethylaminomethyl, or cyclopentadien-5-yl.

In one subgeneric embodiment, the invention provides a compound of Formula VIIIa, VIIb, or VIIIc, or a salt thereof, in which NRₐR_{b} is NHCH₃ or N(CH₃)₂; R₂ is H, and R₃ is acetyl, vinyl, t-butyl, acetoxy, acetamido, methylaminocarbonyl, methanesulfonylamino, methanesulfonyl, propen-2-yl, 2-hydroxyethyl, propyn-1-yl, ethoxy, -chloromethyl, amino, dimethylamino, cyclopentadien-5-yl, vinyl, acetyl, or acetoxy.

In one subgeneric embodiment, the invention provides a compound of Formula VIIIc or a salt thereof, in which NRₐR_{b} is NHCH₃ or N(CH₃)₂; and R₃ and R₅ are, independently, methyl, trifluoromethyl, methoxy, or halo.

In another embodiment, this invention provides a compound of Formula IX or a salt thereof, 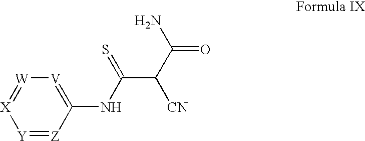
where V-Z as defined as for Formula I.

In one subgeneric embodiment this invention provides a compound of Formula IXa, where V is CR₂, W is CR₃, X is CR₄, Y is CR₅, and Z is CH.

In another subgeneric embodiment the invention provides a compound of Formula IXb, 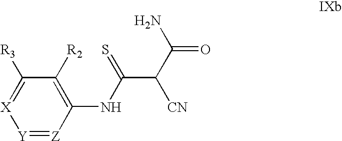
wherein X is N or CR₄, Y is N or CR₅, and Z is N or CH, provided that no more than one of X, Y, and Z is N or CH, where all substituents are as described for Formula I, provided that at least one of R₂-R₅ is other than H.

In another embodiment the invention provides a compound of Formula IX, wherein X is N.

In another embodiment the invention provides a compound of Formula IX or a salt thereof, wherein Y is N.

In another embodiment the invention provides a compound of Formula IX or a salt thereof, wherein Z is N

In another embodiment the invention provides a compound of Formula IX or a salt thereof, wherein none of X, Y, and Z is N.

In a still more specific embodiment the invention provides a compound of Formula IX or a salt thereof, wherein X is N, R₂ and R₃ are both halogen, and R₅ is hydrogen.

In another more specific embodiment, this invention contemplates a compound of Formula IX or a salt thereof, wherein X is N, R₃ is methylamino, dimethylamino, methylaminocarbonyl, ethylaminocarbonyl, dimethylaminocarbonyl, CH₃O(C═O), CH₃CH₂O(C═O), CH₃(C═O)—O—, CH₃CH₂(C═O)—O—, HOCH₂O(C═O), CH₂(OH)CH₂O(C═O), ClCH₂(C═O)—O—, CH₃SO₂—, ClCH₂CH₂(C═O)—O, CH₃(C═O)—NH—, CH₃CH₂(C═O)—NH—, NH₂SO₂—; CH₃NHSO₂—; CH₃SO₂NH—, and R₂ and R₅ are both H.

In another more specific embodiment, this invention contemplates a compound of Formula IX or a salt thereof, wherein X is N, R₃ is hydroxymethyl, 1-hydroxyethyl, 2-hydroxyethyl, propen-3-yl, 1-methylethenyl, or n-propyl, and R₂ and R₅ are both H.

In another embodiment the invention provides a compound of Formula IX or a salt thereof, wherein R₃ is thiazolyl, thienyl, furyl, pyrrolyl, oxazolyl, or imidazolyl.

In another embodiment, this invention provides a compound of Formula IX or a salt thereof, wherein R₃ is cyclopropyl, cyclobutyl, or cyclopentyl.

In another embodiment the invention contemplates a compound of Formula IX or a salt thereof, wherein Y is N, R₃ is thiazol-2-yl, thien-2-yl, thien-3-yl, 2-furyl, 3-furyl, pyrrol-2-yl, isothiazol-2-yl, isoxazol-2-yl, phenyl, pyrrol-3-yl, oxazol-3-yl, 4-methylphenyl, or imidazol-4-yl, or imidazol-2-yl, and R₂ and R₅ are both H.

In another, more specific embodiment, this invention contemplates a compound of Formula IX or a salt thereof, wherein Y is N, R₄ is cyclopropyl, cyclobutyl, cyclopentyl, cyclopent-2-en-yl, cyclopent-1-en-yl, cyclopent-3-en-yl, cyclohexyl, and R₂ and R₃ are both H.

In another, more specific embodiment, this invention contemplates a compound of Formula IX or a salt thereof, wherein Y is N, R₃ is 2-methyl- or 2-chloro-cyclopropyl; 2-methyl- or 2-chloro-cyclobutyl; 2-methyl- or 2-chloro-cyclopentyl; or 2-methyl- or 2-chloro-cyclohexyl; and R₂ and R₄ are both H.

In another more specific embodiment, this invention contemplates a compound of Formula IX or a salt thereof, wherein Y is N, R₃ is hydroxymethyl, 1-hydroxyethyl, 2-hydroxyethyl, propen-3-yl, 1-methylethenyl, or n-propyl, and R₂ and R₄ are both H.

In another, more specific embodiment, this invention contemplates a compound of Formula IX or a salt thereof, wherein Y is N, R₃ is acetyl, n-propanoyl, chloroacetyl, acetoxy, iso-propylcarbonyl, iso-propylcarbonyloxy, 2-chloro-n-propanoyl, 2-chloro-iso-propoxy, (CH₂Cl)₂—CH—, (CH₂Cl)₂—CHO—, 2-methyl- or 2-chloro-cyclopentyl; or 2-methyl- or 2-chloro-cyclohexyl; and R₂ and R₄ are both H.

In another, more specific embodiment, the invention contemplates a compound of Formula IX or a salt thereof, wherein X is N, R₃ is thiazol-2-yl, thien-2-yl, 2-furyl, pyrrol-2-yl, oxazol-2-yl, or imidazol-2-yl, isothiazol-3-yl, isoxazol-3-yl, phenyl, imidazol-4-yl, thiazol-4-yl, thien-3-yl, 3-furyl, pyrrol-3-yl, oxazol-4-yl, isothiazol-4-yl, isoxazol-4-yl, 4-methylphenyl, or imidazol-4-yl, and R₂ and R₅ are both H.

In another embodiment the invention contemplates a compound of Formula IX or a salt thereof, wherein Y is N, R₄ is CH₃C≡C—, phenyl-C≡C—, CH₃CH(OH)C≡C—, CH₃CH₂C≡C—, cyclopropyl-C≡C—, 1-hydroxy-cyclopentyl-C≡C—, 2-hydroxy-cyclopentyl-C≡C—, 3-hydroxy-cyclopentyl-C≡C—, cyclopentyl-C≡C—, isopropyl-C≡C—, tert-butyl-C≡C—, cyclohexyl-C≡C—, cyclohexen-1-yl-C≡C—, or (CH₃)₂NCH₂—C≡C—, and R₂ and R₃ are both H.

In another embodiment, this invention contemplates a compound of Formula IX or a salt thereof, wherein X is N, R₃ is cyclopropyl, cyclobutyl, or cyclopentyl, and R₂ and R₃ are both H.

In another embodiment, this invention contemplates a compound of Formula IX or a salt thereof, where none of X, Y, or Z is N, R₂ is H, and one of R₃, R₄, or R₅ is thiazolyl, isothiazolyl, thienyl, furyl, pyrrolyl, phenyl, oxazolyl, isoxazolyl, or imidazolyl.

In another embodiment, this invention contemplates a compound of Formula IX or a salt thereof, where none of X, Y, or Z is N, R₂ is H, and one of R₃, R₄, or R₅ is methyl, ethyl, n-propyl, cyclopropyl, F, Cl, CF₃, Br, OH, methoxy, nitro, vinyl, acetyl, acetoxy, amino, methylamino, dimethylamino, or CN.

In another embodiment, this invention provides a method of treating Hepatitis C infection in a human comprising providing to a person in need of treatment thereof a therapeutically effective concentration of a compound of Formula I.

In another embodiment, the invention provides a method of treating Hepatitis C infection in a human comprising providing to a person in need of treatment thereof a therapeutically effective concentration of a compound of Formula II.

In another embodiment, the invention provides a method of treating Hepatitis C infection in a human comprising providing to a person in need of treatment thereof a therapeutically effective concentration of a compound of Formula III.

In another embodiment, the invention provides a method of treating Hepatitis C infection in a human comprising providing to a person in need of treatment thereof a therapeutically effective concentration of a compound of Formula IV.

In another embodiment, the invention provides a method of treating Hepatitis C infection in a human comprising providing to a person in need of treatment thereof a therapeutically effective concentration of a compound of Formula V.

The present invention is directed to various substituted isothiazoles as represented in formulas I-VIII, as well as all tautomers and salts thereof, to methods of use of such compounds in the treatment of HCV infection, and to compounds of formula IX, which are synthetic precursors of those isothiazoles. The inventors further contemplate numerous compositions and alternative uses for the compounds according to the inventive subject matter, especially as they relate to compounds, compositions and methods for treatment of diseases in humans.

All prodrugs and metabolites of the compounds according to Formulas I-X are also contemplated. There are numerous prodrug modifications of pharmacologically active molecules known in the art, and all of such modifications are considered suitable for use herein. However, especially preferred prodrugs include those that deliver contemplated compounds to a target cell (e.g., hepatocyte infected with HCV) or target organ (e.g., liver infected with HCV), wherein the prodrug form may be converted within a cell, organ, or other body compartment in an enzymatic or non-enzymatic manner. Further preferred prodrugs particularly include those in which the prodrug form is less active as compared to the corresponding non-prodrug form. Thus, specifically preferred compounds may include a moiety that increases uptake of the prodrug into a cell, or that increases preferential retention of the compound (which may or may not be in prodrug form) in a cell. Alternatively, contemplated compounds may be formulated to increase target specificity of the compound (e.g., organ specific liposomes).

With respect to the metabolite, it should be recognized that metabolites of contemplated compounds may be formed by one or more enzymatic reactions (e.g., via hydrolysis, oxidation, reduction, lyase, or ligase reaction, or even via a polymerase action), or via non-enzymatic reactions (e.g., acid hydrolysis, reduction). For example, a hydrolase or lyase may cleave a portion of contemplated compounds to a more active form.

It is generally contemplated that compounds according to the inventive subject matter may be employed in the treatment of viral diseases, and will advantageously inhibit viral replicase complexes, and especially the RNA dependent RNA polymerase of HCV. However, in further contemplated aspects, diseases caused by a virus other than the HCV may also be treated with the compounds according to the inventive subject matter. For example, contemplated compounds may be effective in treatment of HIV-infected individuals. Yet further contemplated uses of the compounds according to the inventive subject matter include treatment of inflammatory diseases, autoimmune diseases, cancer, diabetes, lupus, infections, and various other viruses.

Thus, in especially preferred aspects of the inventive subject matter, contemplated compounds will have biological activities that include in vitro and in vivo inhibition of HCV RNA replication. It is especially preferred that contemplated compounds may function as a direct inhibitor for HCV replicase complexes, and especially for HCV NS5B polymerase, but may also serve as a prodrug for delivery to a cell infected with a virus, thereby exhibiting further antiviral effect.

The anti-HCV activities of the exemplary compounds were tested in various biological assays, including a cell-based HCV replicon assay, in vitro NS5B polymerase assay, replicase complex assay and cytotoxicity assay.

Of course it should be recognized that use of contemplated compounds is not limited to in vitro systems, and it is particularly contemplated that suitable compounds will be formulated for administration to a mammal, and especially to a human with a condition that is responsive to the administration of such compounds. Therefore, where contemplated compounds are administered in a pharmacological composition, it is contemplated that contemplated compounds can be formulated in admixture with a pharmaceutically acceptable carrier. For example, contemplated compounds can be administered orally as pharmacologically acceptable salts, or intravenously in a physiological saline solution (e.g., buffered to a pH of about 7.2 to 7.5). Conventional buffers such as phosphates, bicarbonates or citrates can be used for this purpose. Of course, one of ordinary skill in the art may modify the formulations within the teachings of the specification to provide numerous formulations for a particular route of administration. In particular, contemplated compounds may be modified to render them more soluble in water or other vehicles, which for example, may be easily accomplished with minor modifications (salt formulation, esterification, etc.) that are well within the ordinary skill in the art. It is also well within the ordinary skill of the art to modify the route of administration and dosage regimen of a particular compound in order to manage the pharmacokinetics of the present compounds for maximum beneficial effect in a patient.

In certain pharmaceutical dosage forms, prodrug forms of contemplated compounds may be formed for various purposes, including reduction of toxicity, increasing the organ or target cell specificity, etc. Among various prodrug forms, acylated (acetylated or other) derivatives, pyridine esters and various salt forms of the present compounds are preferred. One of ordinary skill in the art will recognize how to readily modify the present compounds to prodrug forms to facilitate delivery of active compounds to a target site within the host organism or patient. One of ordinary skill in the art will also take advantage of favorable pharmacokinetic parameters of the prodrug forms, where applicable, in delivering the present compounds to a targeted site within the host organism or patient to maximize the intended effect of the compound.

In addition, contemplated compounds may be administered alone or in combination with other agents for the treatment of various diseases or conditions. Combination therapies according to the present invention comprise the administration of at least one compound of the present invention or a functional derivative thereof and at least one other pharmaceutically active ingredient. Preferred second pharmaceutically active agents for combination therapy include antivirals (e.g., nucleoside analogs or non-nucleoside inhibitors), immune modulators (e.g., cytokines, interferons, histidine, etc.), and/or anti-inflammatory compounds (e.g., steroids). The active ingredient(s) and pharmaceutically active agents may be administered separately or together and when administered separately this may occur simultaneously or separately in any order. Furthermore, the amounts of the active ingredient(s) and pharmaceutically active agent(s) and the relative timings of administration will be selected in order to achieve the desired combined therapeutic effect.

Synthetic Procedures

Compounds listed in Table 1 can be made by the synthetic procedure described in Scheme 1 and US 2004/0039037 A1.

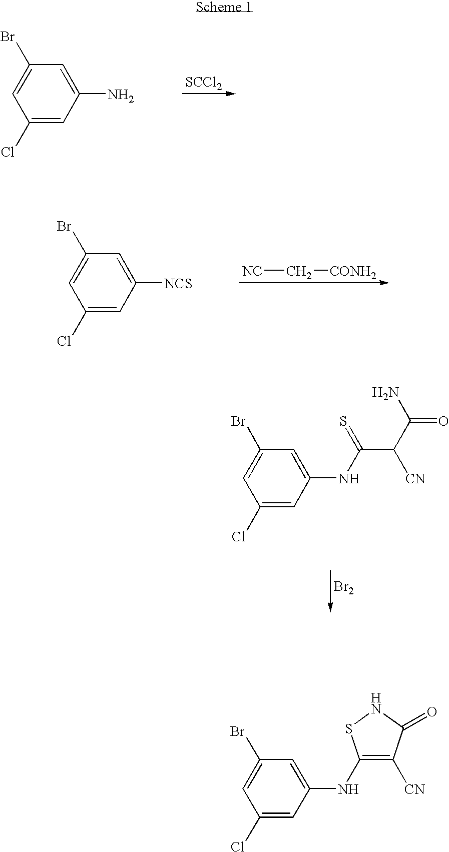

TABLE 1
Prophetic Examples - Compounds accessible using Scheme 1

| | |
|---|---|
| 1. | 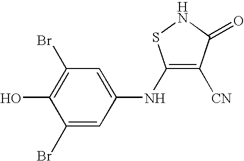 |
| 2. | 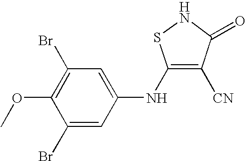 |
| 3. | 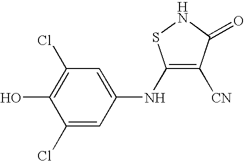 |
| 4. | 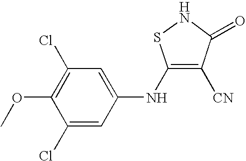 |
| 5. | 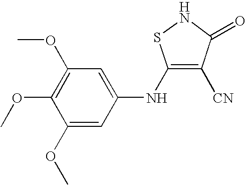 |
| 6. | 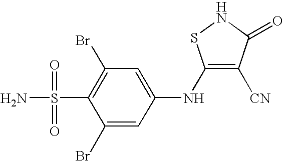 |
| 7. | 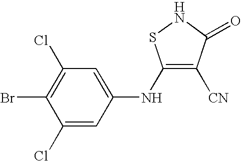 |
| 8. | 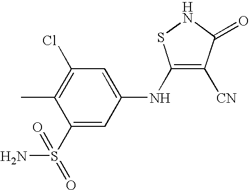 |
| 9. | 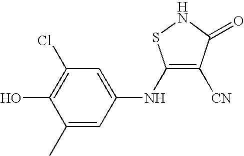 |
| 10. | 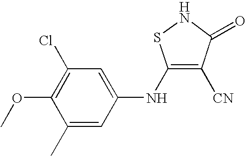 |
| 11. | 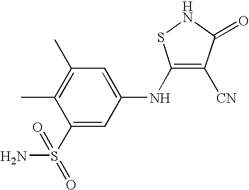 |
| 12. | 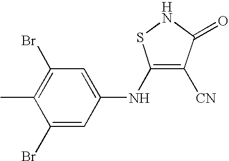 |
| 13. | 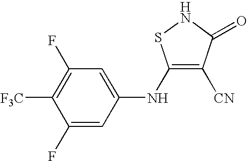 |
| 14. | 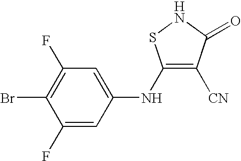 |
| 15. | 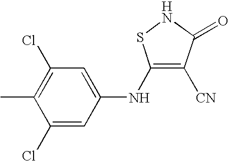 |
| 16. | 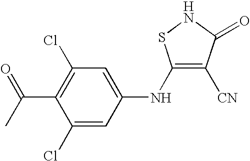 |
| 17. | 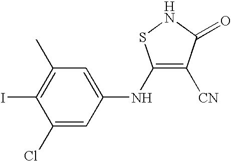 |
| 18. | 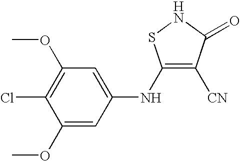 |
| 19. | 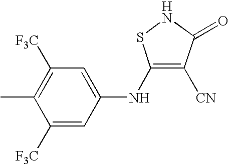 |
| 20. | 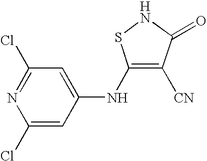 |
| 21. | 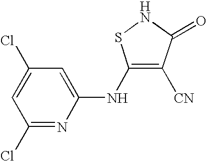 |

Scheme 2 shows how the procedure of Scheme 1 can be extended to produce the additional compounds shown in Table 2. The starting anilines can be prepared using the well-known procedures of Heck, Stille, Suzuki or Sonogashira.

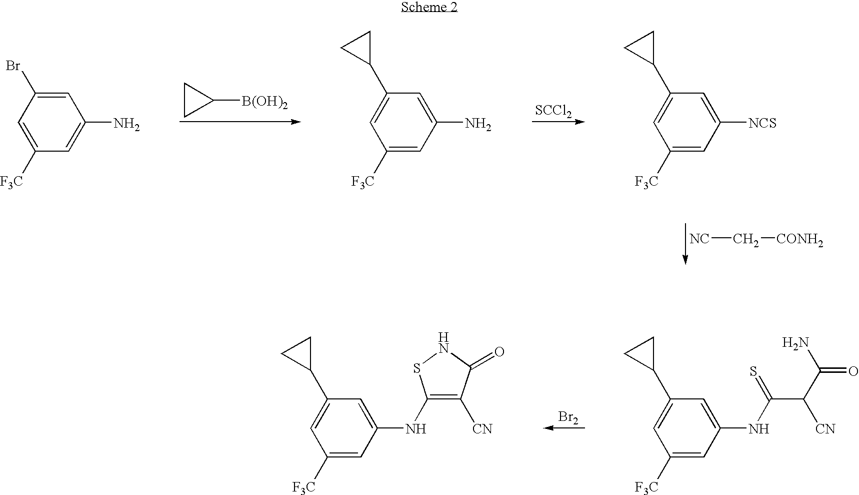

TABLE 2
Prophetic Examples - Compounds accessible using Scheme 2

| | |
|---|---|
| 1. | 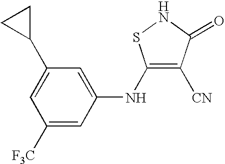 |
| 2. | 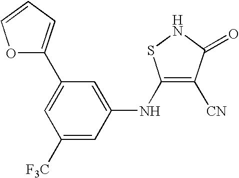 |
| 3. | 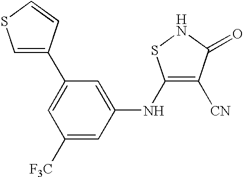 |
| 4. | 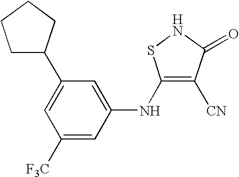 |
| 5. | 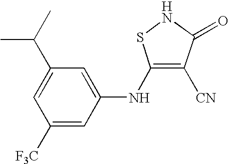 |
| 6. | 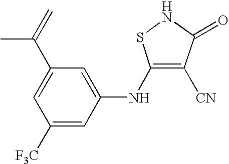 |
| 7. | 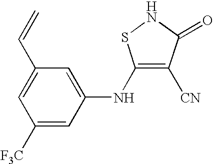 |
| 8. | 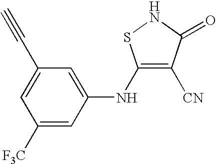 |
| 9. | 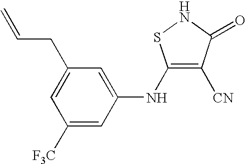 |
| 10. | 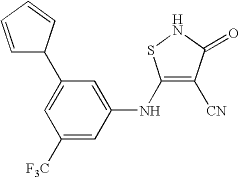 |
| 11. | 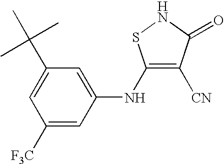 |
| 12. | 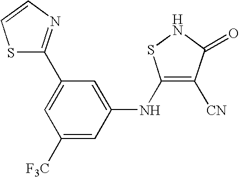 |
| 13. | 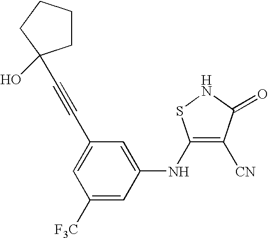 |
| 14. | 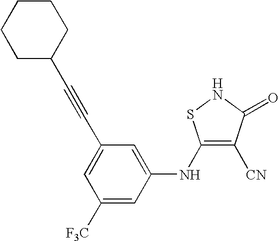 |
| 15. | 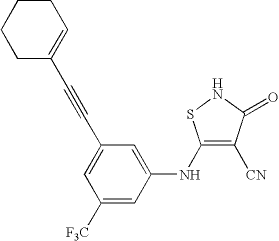 |
| 16. | 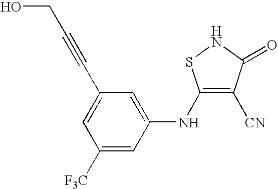 |
| 17. | 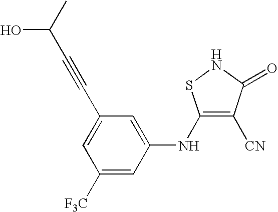 |
| 18. | 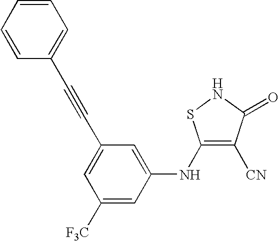 |
| 19. | 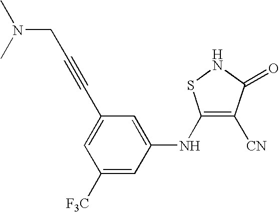 |
| 20. | 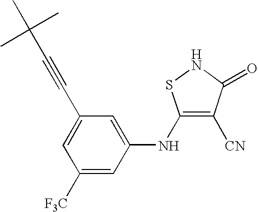 |
| 21. | 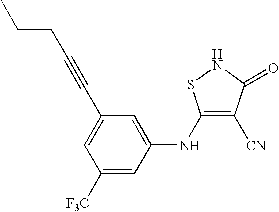 |
| 22. | 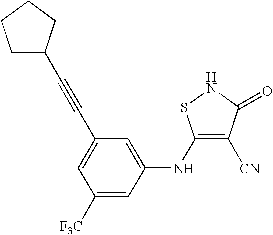 |
| 23. | 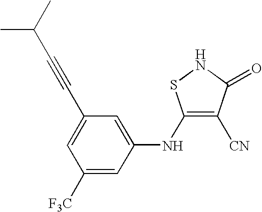 |
| 24. | 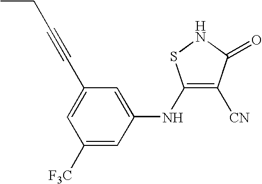 |
| 25. | 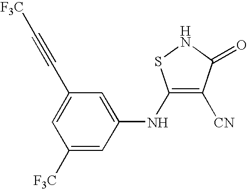 |
| 26. | 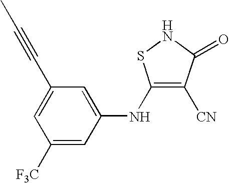 |
| 27. | 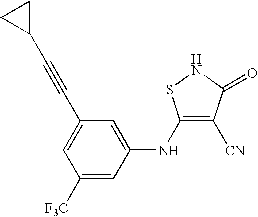 |
| 28. | 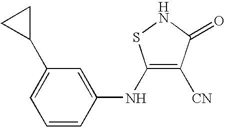 |
| 29. | 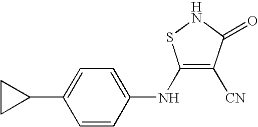 |
| 30. | 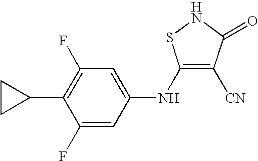 |
| 31. | 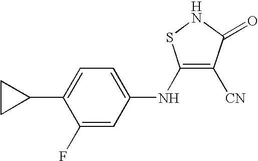 |
| 32. | 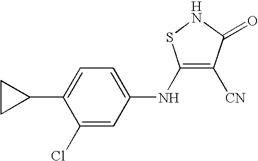 |
| 33. | 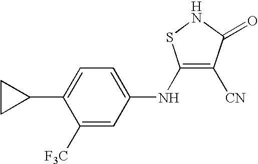 |

Scheme 3 describes an alternative route to many of the compounds of this invention, particularly those shown in Table 3. See Brown D. W. et al., Science of Synthesis 2002, 11, 507; Wolfe P. J. et al., J. Org. Chem. 2000, 65, 1158. A benzyl bromide can be used in place of the phenyl bromide in the final step, to produce a 5-benzylamino isothiazole.

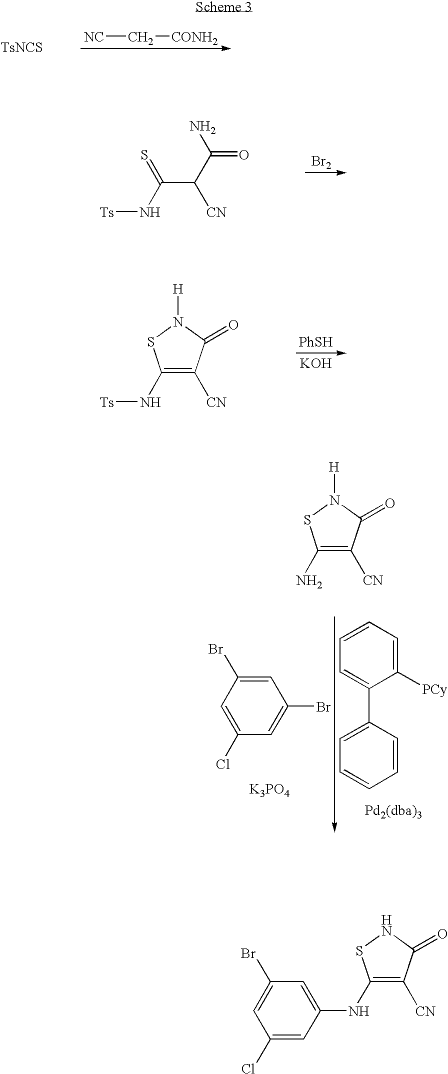

TABLE 3
Prophetic Examples - Compounds accessible using Scheme 3

| | |
|---|---|
| 1. | 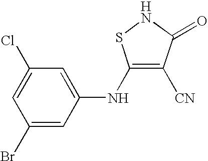 |
| 2. | 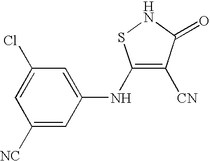 |
| 3. | 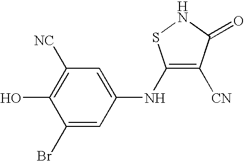 |
| 4. | 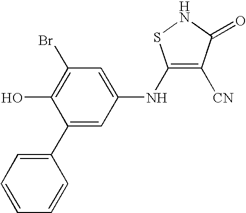 |
| 5. | 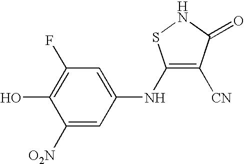 |
| 6. | 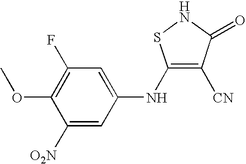 |
| 7. | 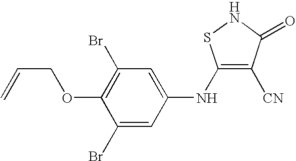 |
| 8. | 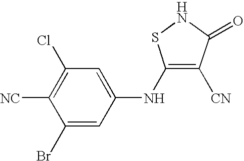 |
| 9. | 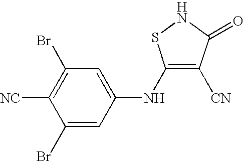 |
| 10. | 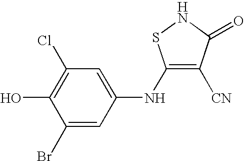 |
| 11. | 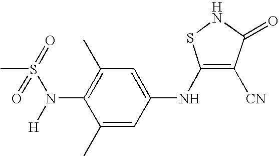 |
| 12. | 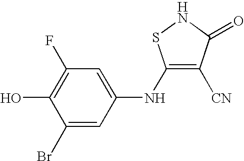 |
| 13. | 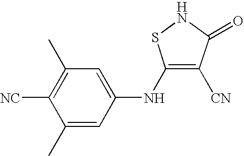 |
| 14. | 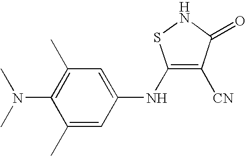 |
| 15. | 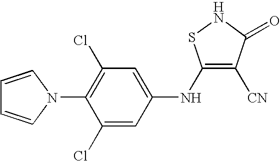 |
| 16. | 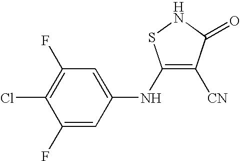 |
| 17. | 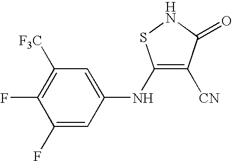 |
| 18. | 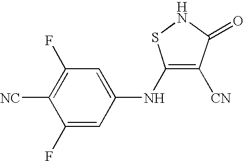 |
| 19. | 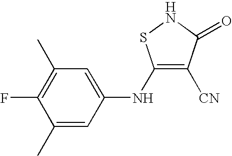 |
| 20. | 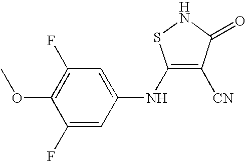 |
| 21. | 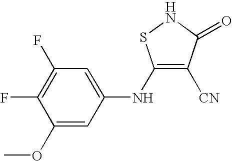 |
| 22. | 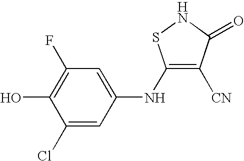 |
| 23. | 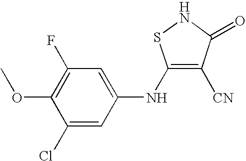 |
| 24. | 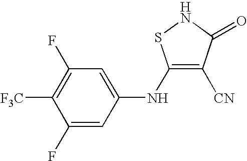 |
| 25. | 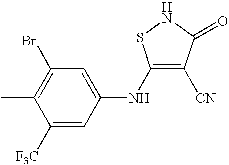 |
| 26. | 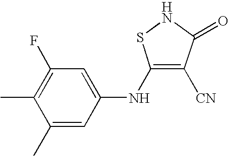 |
| 27. | 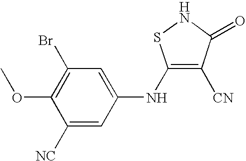 |

TABLE 4
Contemplated and Prophetic Examples of Pyrimidylamino Isothiazoles

| | |
|---|---|
| 1. | 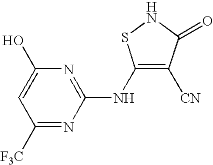 |
| 2. | 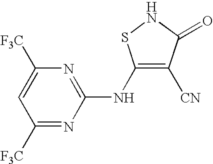 |
| 3. | 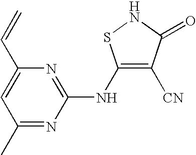 |
| 4. | 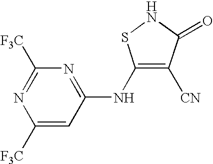 |
| 5. | 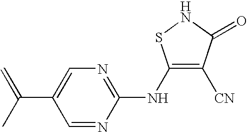 |
| 6. | 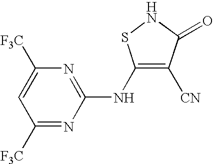 |
| 7. | 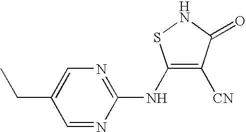 |
| 8. | 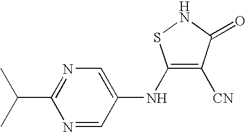 |
| 9. | 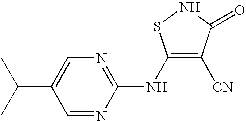 |
| 10. | 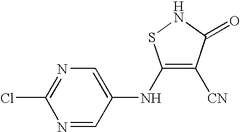 |
| 11. | 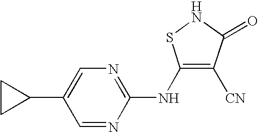 |
| 12. | 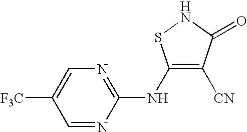 |
| 13. | 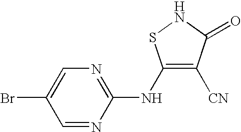 |
| 14. | 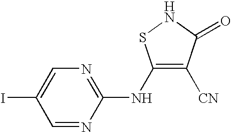 |
| 15. | 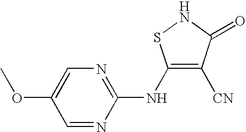 |
| 16. | 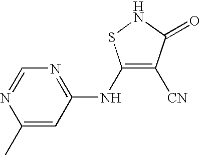 |
| 17. | 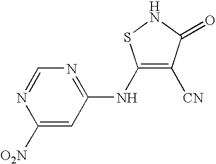 |
| 18. | 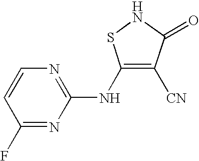 |
| 19. | 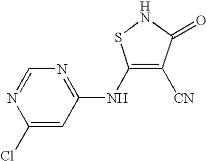 |
| 20. | 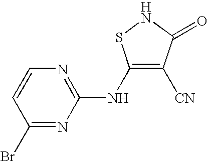 |
| 21. | 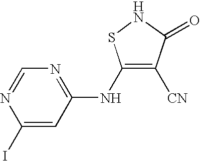 |
| 22. | 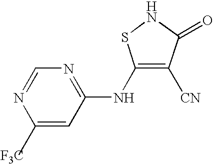 |
| 23. | 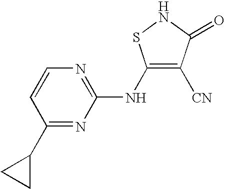 |
| 24. | 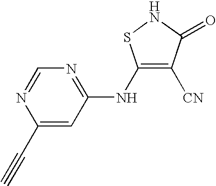 |
| 25. | 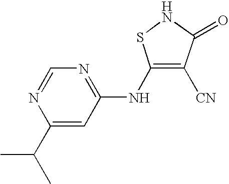 |
| 26. | 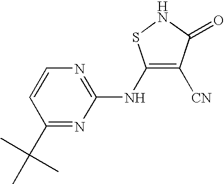 |
| 27. | 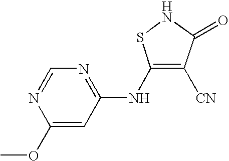 |
| 28. | 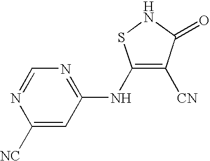 |
| 29. | 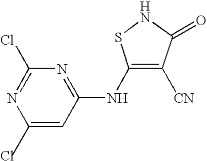 |
| 30. | 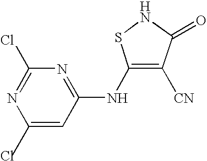 |
| 31. | 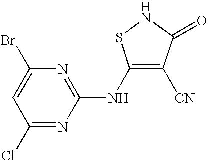 |
| 32. | 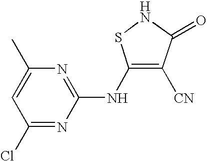 |
| 33. | 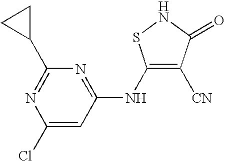 |
| 34. | 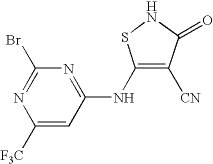 |
| 35. | 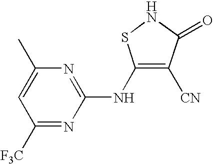 |
| 36. | 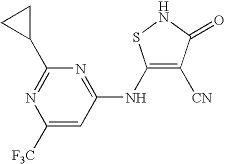 |
| 37. | 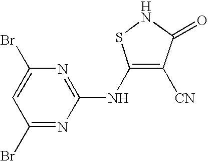 |
| 38. | 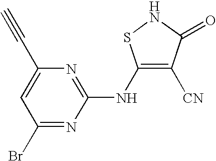 |
| 39. | 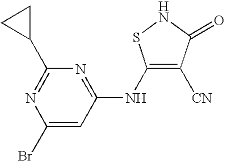 |
| 40. | 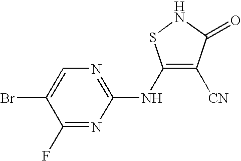 |
| 41. | 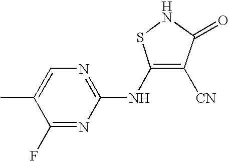 |
| 42. | 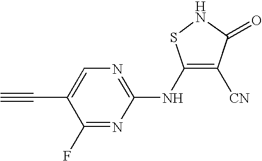 |
| 43. | 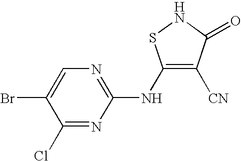 |
| 44. | 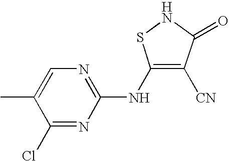 |
| 45. | 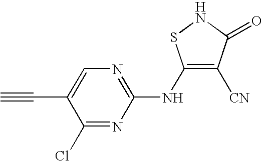 |
| 46. | 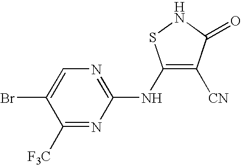 |
| 47. | 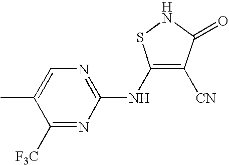 |
| 48. | 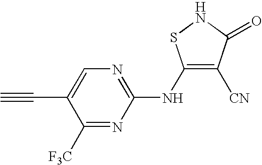 |

TABLE 5
Further Contemplated and Prophetic Examples of Pyrimidyl Isothiazoles

| | |
|---|---|
| 1. | 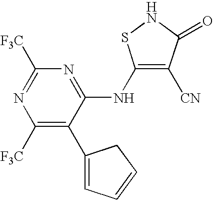 |
| 2. | 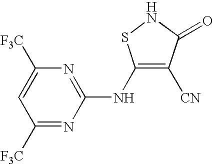 |
| 3. | 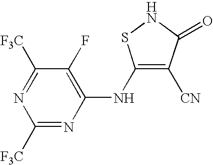 |
| 4. | 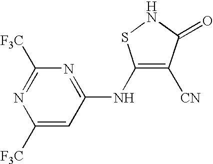 |
| 5. | 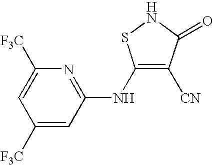 |
| 6. | 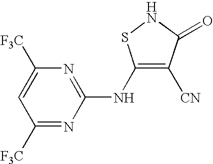 |
| 7. | 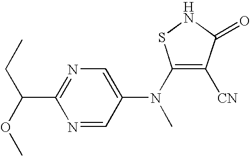 |
| 8. | 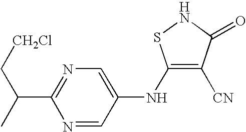 |
| 9. | 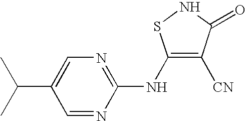 |
| 10. | 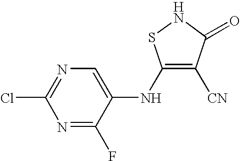 |
| 11. | 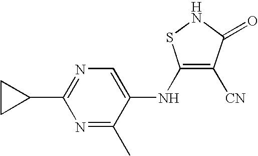 |
| 12. | 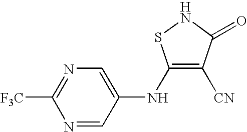 |
| 13. | 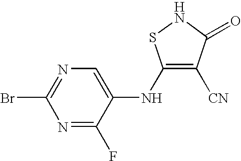 |
| 14. | 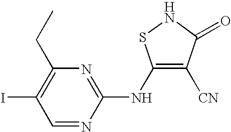 |
| 15. | 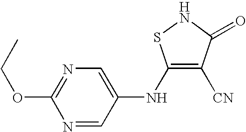 |
| 16. | 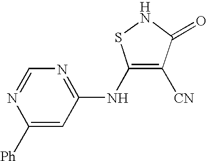 |
| 17. | 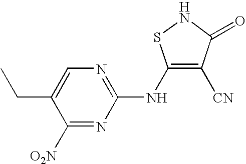 |
| 18. | 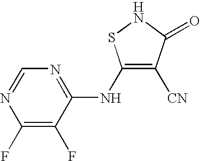 |
| 19. | 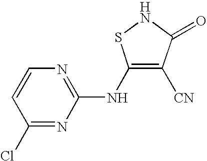 |
| 20. | 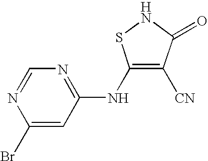 |
| 21. | 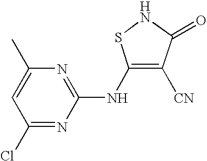 |
| 22. | 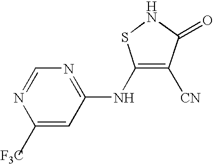 |
| 23. | 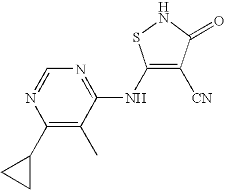 |
| 24. | 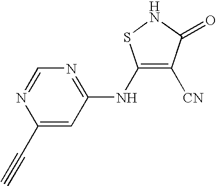 |
| 25. | 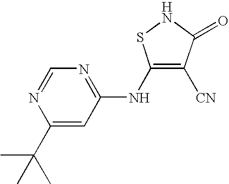 |
| 26. | 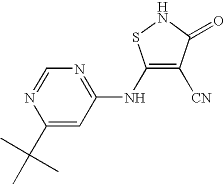 |
| 27. | 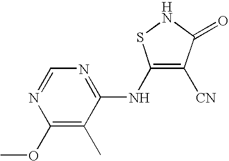 |
| 28. | 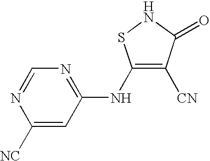 |
| 29. | 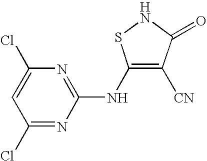 |
| 30. | 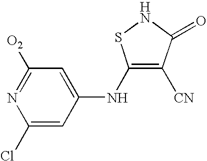 |
| 31. | 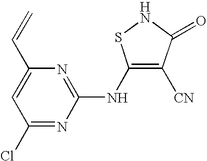 |
| 32. | 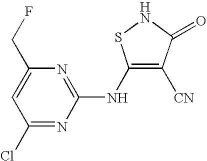 |
| 33. | 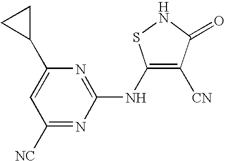 |
| 34. | 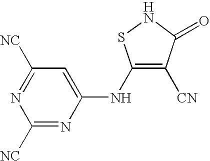 |
| 35. | 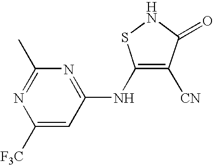 |
| 36. | 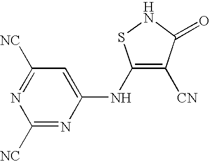 |
| 37. | 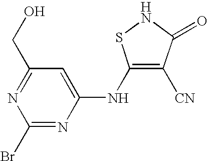 |
| 38. | 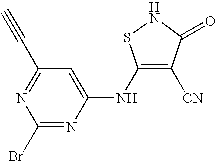 |
| 39. | 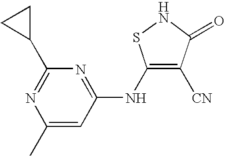 |

TABLE 6
Prophetic Examples of Compounds of Formula IIIa

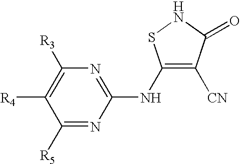

| Com- | | | |
|---|---|---|---|
| pound # | R₃ = | R₄ = | R₅ = |
| 1 | Br | OH | Br |
| 2 | Br | OMe | Br |
| 3 | Cl | OH | Cl |
| 4 | Cl | OMe | Cl |
| 5 | OMe | OMe | OMe |
| 6 | Br | H₂N—SO₂— | Br |
| 7 | Cl | CH₃C(═O)NH | H |
| 8 | Cl | Me | H₂N—SO₂— |
| 9 | Cl | OH | Me |
| 10 | Cl | OMe | Me |
| 11 | Me | Me | H₂N—SO₂— |
| 12 | Br | Me | Br |
| 13 | F | CF₃ | F |
| 14 | F | Br | F |
| 15 | Cl | Me | Cl |
| 16 | Cl | CH₃C(═O)O | H |
| 17 | Me | I | Cl |
| 18 | OMe | Cl | OMe |
| 19 | CF₃ | Me | CF₃ |
| 20 | Cl | H | Cl |
| 21 | Cl | I | H |
| 22 | Me | Me—C≡C | H |
| 23 | isothiazol-3-yl | H | CF₃ |
| 24 | 1-OH-cyclopentyl-C≡C— | H | CF₃ |
| 25 | cyclohexyl-C≡C— | H | CF₃ |
| 26 | cyclohexen-1-yl-C≡C— | H | CF₃ |
| 27 | HOCH₂—C≡C— | H | CF₃ |
| 28 | CH₃CH(OH)C≡C— | H | CF₃ |
| 29 | C₆H₅—C≡C— | H | CF₃ |
| 30 | CH₃C(═O)O | H | CN |

TABLE 7
Prophetic Examples of Thiocarbamoyl Precursors of Compounds of
Formula IIIa

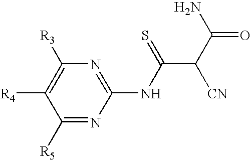

| Com- | | | |
|---|---|---|---|
| pound # | R₃ = | R₄ = | R₅ = |
| 31 | Br | OH | Br |
| 32 | Br | OMe | Br |
| 33 | Cl | OH | Cl |
| 34 | Cl | OMe | Cl |
| 35 | OMe | OMe | OMe |
| 36 | Br | H₂N—SO₂— | Br |
| 37 | Cl | CH₃C(═O)NH | H |
| 38 | Cl | Me | H₂N—SO₂— |
| 39 | Cl | OH | Me |
| 40 | Cl | OMe | Me |
| 41 | Me | Me | H₂N—SO₂— |
| 42 | Br | Me | Br |
| 43 | F | CF₃ | F |
| 44 | F | Br | F |
| 45 | Cl | Me | Cl |
| 46 | Cl | CH₃C(═O)O | H |
| 47 | Me | I | Cl |
| 48 | OMe | Cl | OMe |
| 49 | CF₃ | Me | CF₃ |
| 50 | Cl | H | Cl |
| 51 | Cl | I | H |
| 52 | Me | Me—C≡C | H |
| 53 | isothiazol-3-yl | H | CF₃ |
| 54 | 1-OH-cyclopentyl-C≡C— | H | CF₃ |
| 55 | cyclohexyl-C≡C— | H | CF₃ |
| 56 | cyclohexen-1-yl-C≡C— | H | CF₃ |
| 57 | HOCH₂—C≡C— | H | CF₃ |
| 58 | CH₃CH(OH)C≡C— | H | CF₃ |
| 59 | C₆H₅—C≡C— | H | CF₃ |
| 60 | H | thien-3-yl | F |

TABLE 8
Prophetic Examples of Compounds of Formula IIIb

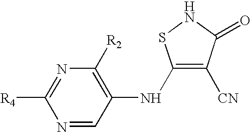

| Compound # | R₂ = | R₄ = |
|---|---|---|
| 61 | H | OH |
| 62 | H | OMe |
| 63 | Me | OH |
| 64 | Cl | OMe |
| 65 | H | OMe |
| 66 | H | H₂N—SO₂— |
| 67 | Cl | CH₃C(═O)NH |
| 68 | Cl | H₂N—SO₂— |
| 69 | Cl | OH |
| 70 | Cl | OMe |
| 71 | Me | Me |
| 72 | F | F |
| 73 | F | CF₃ |
| 74 | F | Br |
| 75 | Cl | Me |
| 76 | H | CH₃C(═O)O |
| 77 | Me | Me—C≡C |
| 78 | H | isothiazol-3-yl |
| 79 | 1-OH-cyclopentyl-C≡C— | H |
| 80 | cyclohexyl-C≡C— | H |
| 81 | cyclohexen-1-yl-C≡C— | H |
| 82 | HOCH₂—C≡C— | H |
| 83 | CH₃CH(OH)C≡C— | H |
| 84 | C₆H₅—C≡C— | H |
| 85 | CH₃C(═O)O | H |

TABLE 9
Prophetic Examples of Thiocarbamoyl Precursors to Compounds of
Formula IIIb

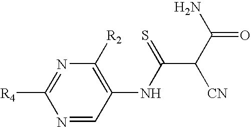

| Compound # | R₂ = | R₄ = |
|---|---|---|
| 86 | H | OH |
| 87 | H | OMe |
| 88 | Me | OH |
| 89 | Cl | OMe |
| 90 | H | OMe |
| 91 | H | H₂N—SO₂— |
| 92 | Cl | CH₃C(═O)NH |
| 93 | Cl | H₂N—SO₂— |
| 94 | Cl | OH |
| 95 | Cl | OMe |
| 96 | Me | Me |
| 97 | F | F |
| 98 | F | CF₃ |
| 99 | F | Br |
| 100 | Cl | Me |
| 101 | H | CH₃C(═O)O |
| 102 | Me | Me—C≡C |
| 103 | H | isothiazol-3-yl |
| 104 | 1-OH-cyclopentyl-C≡C— | H |
| 105 | cyclohexyl-C≡C— | H |
| 106 | cyclohexen-1-yl-C≡C— | H |
| 107 | HOCH₂—C≡C— | H |
| 108 | CH₃CH(OH)C≡C— | H |
| 109 | C₆H₅—C≡C— | H |
| 110 | CH₃C(═O)O | H |

TABLE 10
Prophetic Examples of Compounds of Formula IIIc

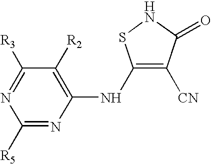

| Compound # | R₂ = | R₃ = | R₅ = |
|---|---|---|---|
| 111 | H | OH | Br |
| 112 | H | OMe | Br |
| 113 | Cl | OH | Cl |
| 114 | Cl | OMe | Cl |
| 115 | H | OMe | OMe |
| 116 | H | H₂N—SO₂— | Br |
| 117 | H | CH₃C(═O)NH | H |
| 118 | Cl | Me | H₂N—SO₂— |
| 119 | Cl | OH | Me |
| 120 | Cl | OMe | Me |
| 121 | Me | Me | H₂N—SO₂— |
| 122 | Br | Me | Br |
| 123 | F | CF₃ | F |
| 124 | F | Br | F |
| 125 | Cl | Me | Cl |
| 126 | Cl | CH₃C(═O)O | H |
| 127 | Me | I | Cl |
| 128 | OMe | Cl | OMe |
| 129 | CF₃ | Me | CF₃ |
| 130 | H | HOCH₂—C≡C— | H |
| 131 | H | CH₃CH(OH)C≡C— | CF₃ |
| 132 | H | C₆H₅—C≡C— | CF₃ |
| 133 | CH₃OC(═O)— | H | CN |

TABLE 11
Prophetic Examples of Thiocarbamoyl Precursors to Compounds of
Formula IIIc

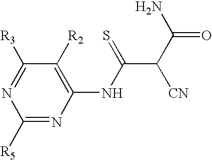

| Compound # | R₂ = | R₃ = | R₅ = |
|---|---|---|---|
| 134 | H | OH | Br |
| 135 | H | OMe | Br |
| 136 | Cl | OH | Cl |
| 137 | Cl | OMe | Cl |
| 138 | H | OMe | OMe |
| 139 | H | H₂N—SO₂— | Br |
| 140 | H | CH₃C(═O)NH | H |
| 141 | Cl | Me | H₂N—SO₂— |
| 142 | Cl | OH | Me |
| 143 | Cl | OMe | Me |
| 144 | Me | Me | H₂N—SO₂— |
| 145 | Br | Me | Br |
| 146 | F | CF₃ | F |
| 147 | F | Br | F |
| 148 | Cl | Me | Cl |
| 149 | Cl | CH₃C(═O)O | H |
| 150 | Me | I | Cl |
| 151 | OMe | Cl | OMe |
| 152 | CF₃ | Me | CF₃ |
| 153 | H | HOCH₂—C≡C— | H |
| 154 | H | CH₃CH(OH)C≡C— | CF₃ |
| 155 | H | C₆H₅—C≡C— | CF₃ |
| 156 | CH₃OC(═O)— | H | CN |

TABLE 8
Prophetic Examples of Compounds of Formula IIc

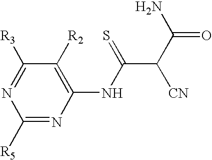

| Compound # | R₂ = | R₃ = | R₅ = |
|---|---|---|---|
| 157 | H | OH | Br |
| 158 | H | OMe | Br |
| 159 | Cl | OH | Cl |
| 160 | Cl | OMe | Cl |
| 161 | H | OMe | OMe |
| 162 | H | H₂N—SO₂— | Br |
| 163 | H | CH₃C(═O)NH | H |
| 164 | Cl | Me | H₂N—SO₂— |
| 165 | Cl | OH | Me |
| 166 | Cl | OMe | Me |
| 167 | Me | Me | H₂N—SO₂— |
| 168 | Br | Me | Br |
| 169 | F | CF₃ | F |
| 170 | F | Br | F |
| 171 | Cl | Me | Cl |
| 172 | Cl | CH₃C(═O)O | H |
| 173 | Me | I | Cl |
| 174 | OMe | Cl | OMe |
| 175 | CF₃ | Me | CF₃ |
| 176 | H | HOCH₂—C≡C— | H |
| 177 | H | CH₃CH(OH)C≡C— | CF₃ |
| 178 | H | C₆H₅—C≡C— | CF₃ |
| 179 | CH₃OC(═O)— | H | CN |

Compounds of Formulas IV, V, and VI, shown in tables below, can be prepared using standard procedures from the corresponding 4-isocyanato isothiazoles, as shown in Scheme 4. As those skilled in the art will note, the hydroxy group is protected until the final step. 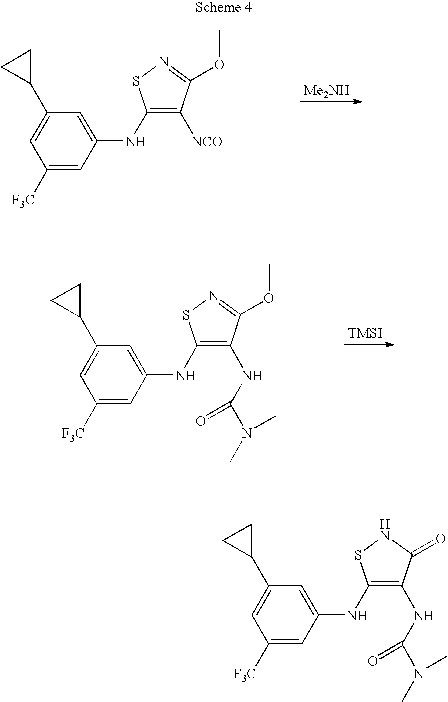
The tables below show a wide variety of prophetic examples.

Additional prophetic examples are shown in the following tables

TABLE 9

| | |
|---|---|
| 1. | 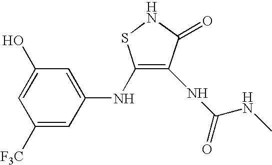 |
| 2. | 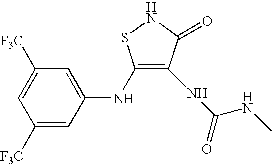 |
| 3. | 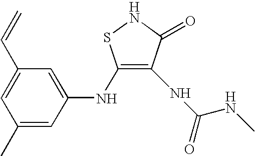 |
| 4. | 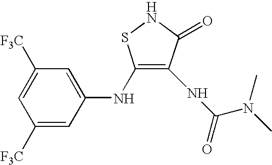 |
| 5. | 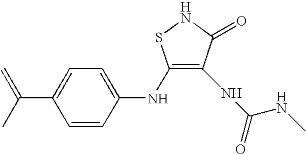 |
| 6. | 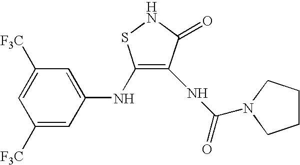 |
| 7. | 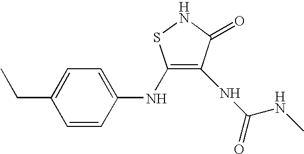 |
| 8. | 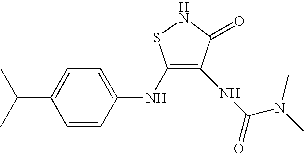 |
| 9. | 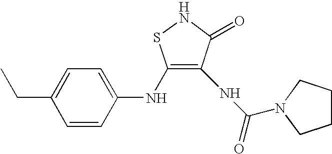 |
| 10. | 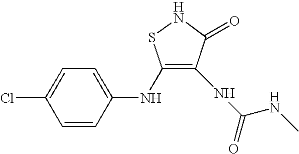 |
| 11. | 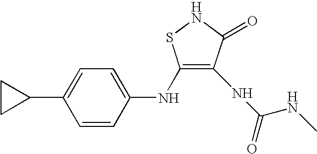 |
| 12. | 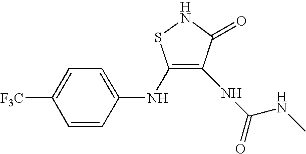 |
| 13. | 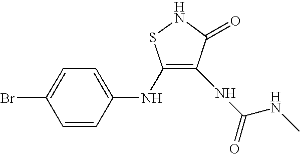 |
| 14. | 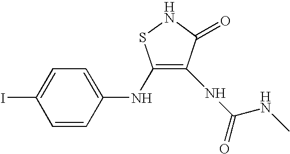 |
| 15. | 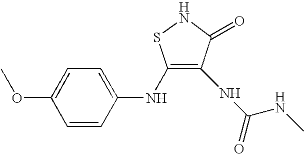 |
| 16. | 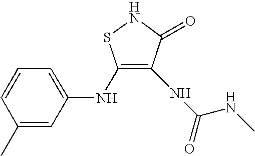 |
| 17. | 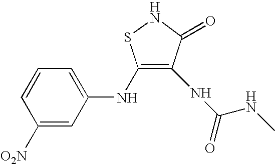 |
| 18. | 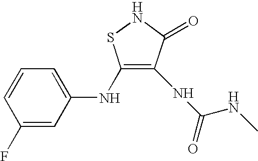 |
| 19. | 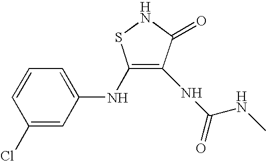 |
| 20. | 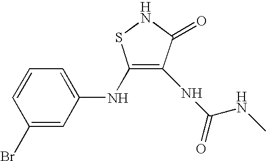 |
| 21. | 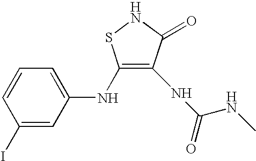 |
| 22. | 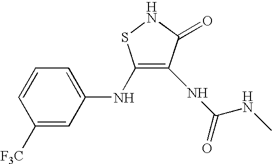 |
| 23. | 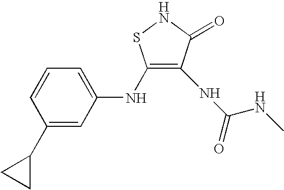 |
| 24. | 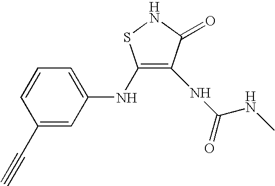 |
| 25. | 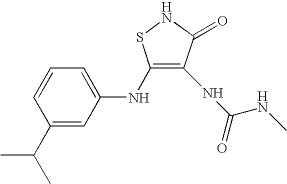 |
| 26. | 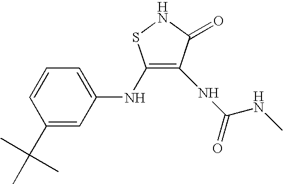 |
| 27. | 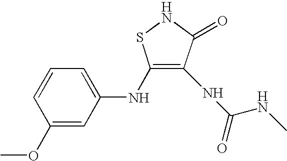 |
| 28. | 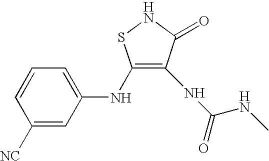 |
| 29. | 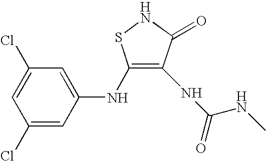 |
| 30. | 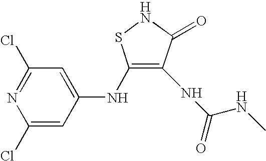 |
| 31. | 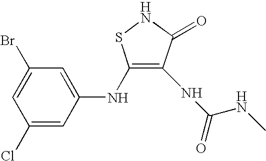 |
| 32. | 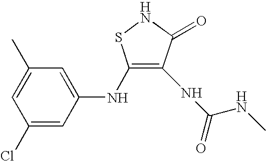 |
| 33. | 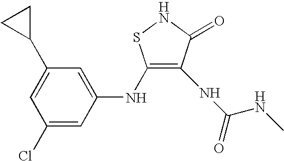 |
| 34. | 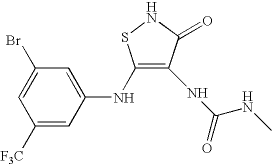 |
| 35. | 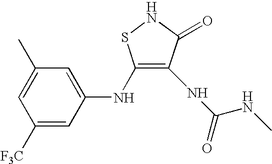 |
| 36. | 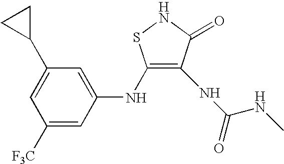 |
| 37. | 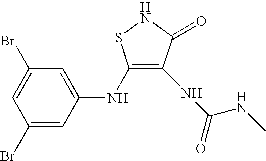 |
| 38. | 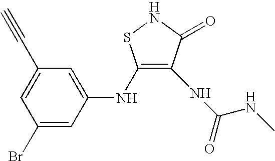 |
| 39. | 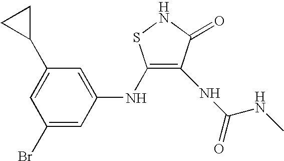 |
| 40. | 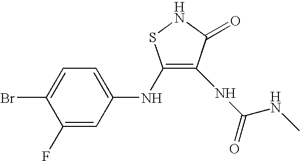 |
| 41. | 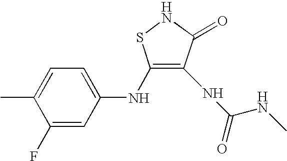 |
| 42. | 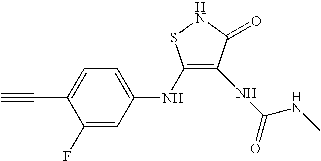 |
| 43. | 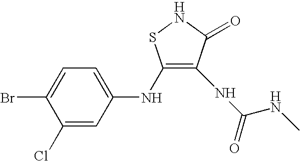 |
| 44. | 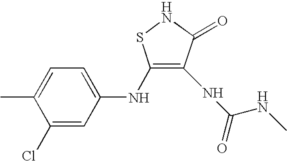 |
| 45. | 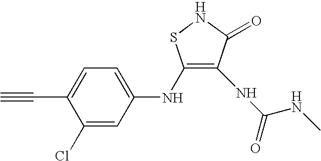 |
| 46. | 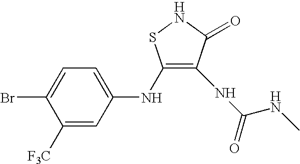 |
| 47. | 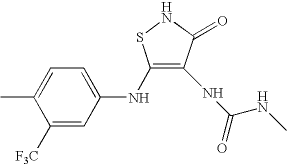 |
| 48. | 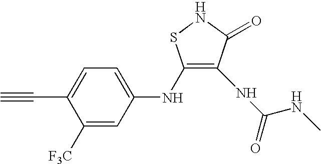 |

TABLE 10

| | |
|---|---|
| 1. | 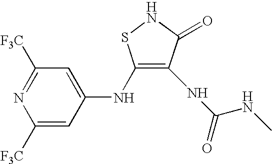 |
| 2. | 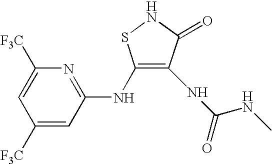 |
| 3. | 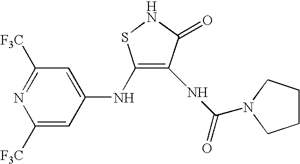 |
| 4. | 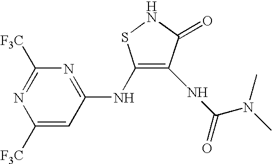 |
| 5. | 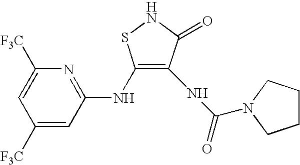 |
| 6. | 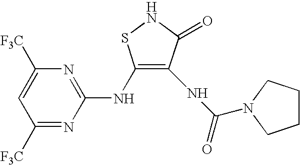 |
| 7. | 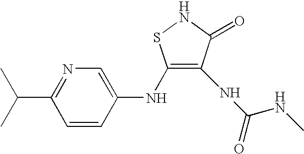 |
| 8. | 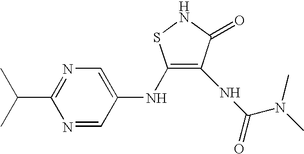 |
| 9. | 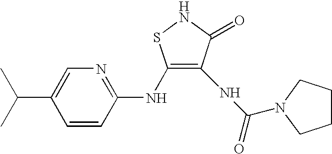 |
| 10. | 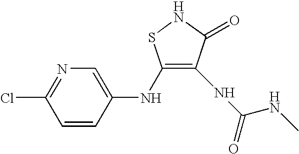 |
| 11. | 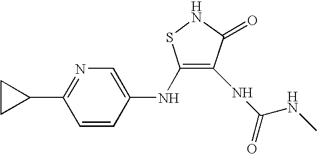 |
| 12. | 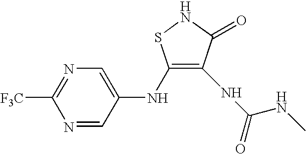 |
| 13. | 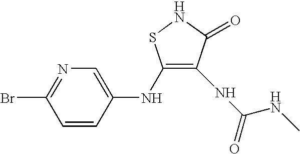 |
| 14. | 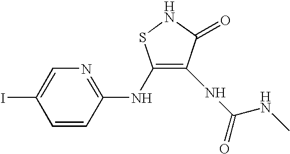 |
| 15. | 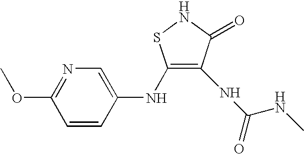 |
| 16. | 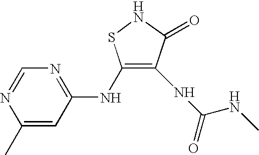 |
| 17. | 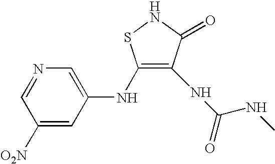 |
| 18. | 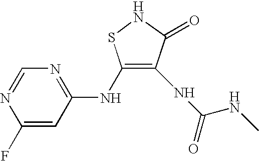 |
| 19. | 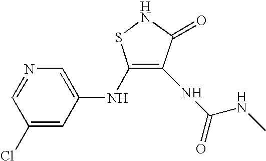 |
| 20. | 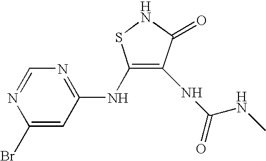 |
| 21. | 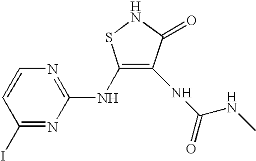 |
| 22. | 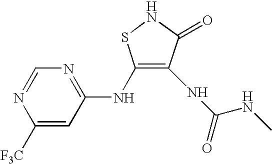 |
| 23. | 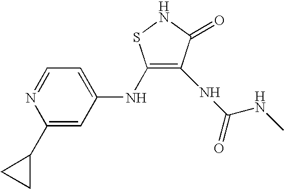 |
| 24. | 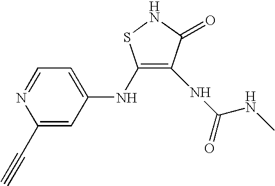 |
| 25. | 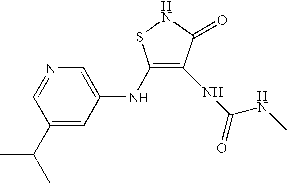 |
| 26. | 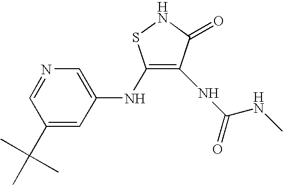 |
| 27. | 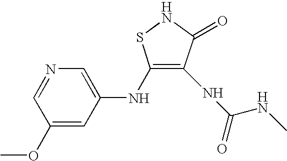 |
| 28. | 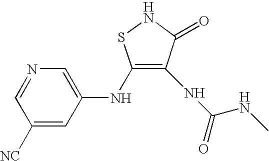 |
| 29. | 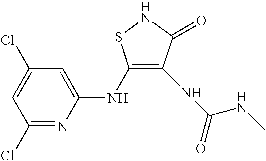 |
| 30. | 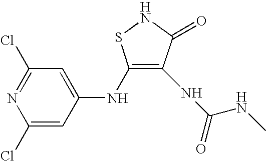 |
| 31. | 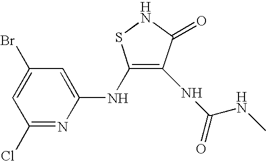 |
| 32. | 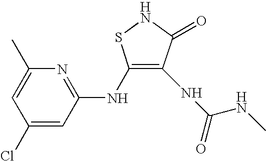 |
| 33. | 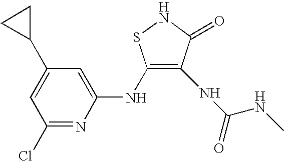 |
| 34. | 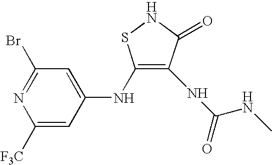 |
| 35. | 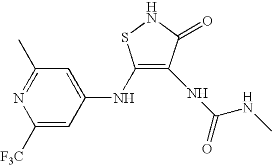 |
| 36. | 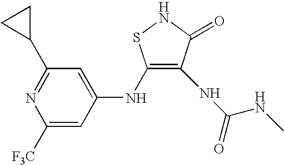 |
| 37. | 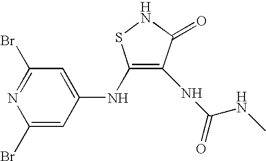 |
| 38. | 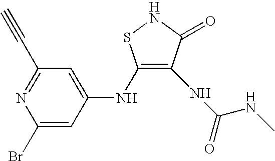 |
| 39. | 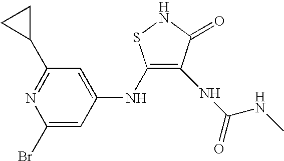 |
| 40. | 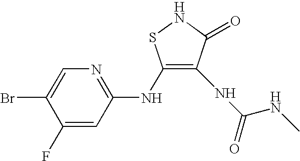 |
| 41. | 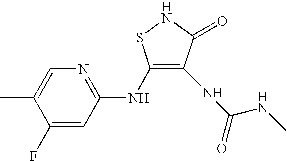 |
| 42. | 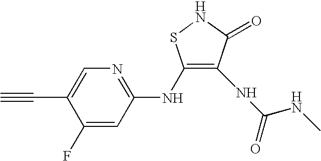 |
| 43. | 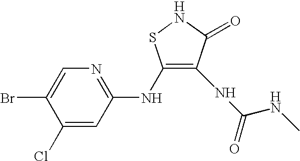 |
| 44. | 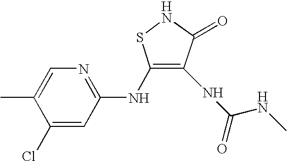 |
| 45. | 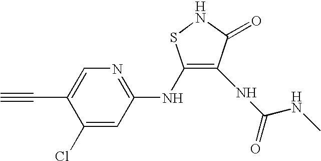 |
| 46. | 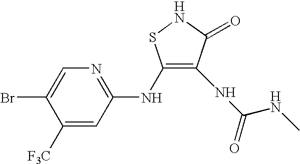 |
| 47. | 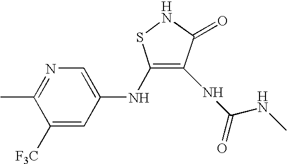 |
| 48. | 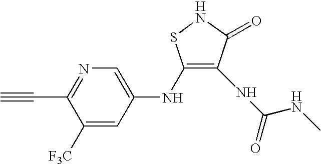 |

Additional contemplated compounds and prophetic examples are shown in the tables below.

TABLE 11
Prophetic Examples of Compounds

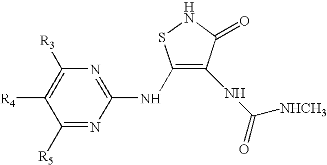

| Com- | | | |
|---|---|---|---|
| pound # | R₃ = | R₄ = | R₅ = |
| 1 | Br | OH | Br |
| 2 | Br | OMe | Br |
| 3 | Cl | OH | Cl |
| 4 | Cl | OMe | Cl |
| 5 | OMe | OMe | OMe |
| 6 | Br | H₂N—SO₂— | Br |
| 7 | Cl | CH₃C(═O)NH | H |
| 8 | Cl | Me | H₂N—SO₂— |
| 9 | Cl | OH | Me |
| 10 | Cl | OMe | Me |
| 11 | Me | Me | H₂N—SO₂— |
| 12 | Br | Me | Br |
| 13 | F | CF₃ | F |
| 14 | F | Br | F |
| 15 | Cl | Me | Cl |
| 16 | Cl | CH₃C(═O)O | H |
| 17 | Me | I | Cl |
| 18 | OMe | Cl | OMe |
| 19 | CF₃ | Me | CF₃ |
| 20 | Cl | H | Cl |
| 21 | Cl | I | H |
| 22 | Me | Me—C≡C | H |
| 23 | isothiazol-2-yl | H | CF₃ |
| 24 | 1-OH-cyclopentyl-C≡C— | H | CF₃ |
| 25 | cyclohexyl-C≡C— | H | CF₃ |
| 26 | cyclohexen-1-yl-C≡C— | H | CF₃ |
| 27 | HOCH₂—C≡C— | H | CF₃ |
| 28 | CH₃CH(OH)C≡C— | H | CF₃ |
| 29 | C₆H₅—C≡C— | H | CF₃ |
| 30 | CH₃C(═O)O | H | CN |

TABLE 12
Prophetic Examples of Compounds

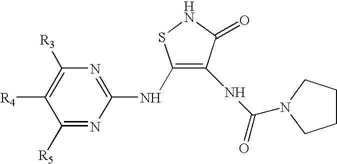

| Com- | | | |
|---|---|---|---|
| pound # | R₃ = | R₄ = | R₅ = |
| 31 | Br | OH | Br |
| 32 | Br | OMe | Br |
| 33 | Cl | OH | Cl |
| 34 | Cl | OMe | Cl |
| 35 | OMe | OMe | OMe |
| 36 | Br | H₂N—SO₂— | Br |
| 37 | Cl | CH₃C(═O)NH | H |
| 38 | Cl | Me | H₂N—SO₂— |
| 39 | Cl | OH | Me |
| 40 | Cl | OMe | Me |
| 41 | Me | Me | H₂N—SO₂— |
| 42 | Br | Me | Br |
| 43 | F | CF₃ | F |
| 44 | F | Br | F |
| 45 | Cl | Me | Cl |
| 46 | Cl | CH₃C(═O)O | H |
| 47 | Me | I | Cl |
| 48 | OMe | Cl | OMe |
| 49 | CF₃ | Me | CF₃ |
| 50 | Cl | H | Cl |
| 51 | Cl | I | H |
| 52 | Me | Me—C≡C | H |
| 53 | isothiazol-2-yl | H | CF₃ |
| 54 | 1-OH-cyclopentyl-C≡C— | H | CF₃ |
| 55 | cyclohexyl-C≡C— | H | CF₃ |
| 56 | cyclohexen-1-yl-C≡C— | H | CF₃ |
| 57 | HOCH₂—C≡C— | H | CF₃ |
| 58 | CH₃CH(OH)C≡C— | H | CF₃ |
| 59 | C₆H₅—C≡C— | H | CF₃ |
| 60 | H | thien-3-yl | F |

TABLE 13
Prophetic Examples of Compounds

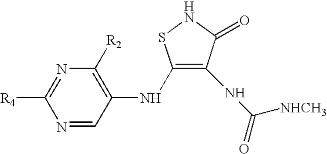

| Compound # | R₂ = | R₄ = |
|---|---|---|
| 61 | H | OH |
| 62 | H | OMe |
| 63 | Me | OH |
| 64 | Cl | OMe |
| 65 | H | OMe |
| 66 | H | H₂N—SO₂— |
| 67 | Cl | CH₃C(═O)NH |
| 68 | Cl | H₂N—SO₂— |
| 69 | Cl | OH |
| 70 | Cl | OMe |
| 71 | Me | Me |
| 72 | F | F |
| 73 | F | CF₃ |
| 74 | F | Br |
| 75 | Cl | Me |
| 76 | H | CH₃C(═O)O |
| 77 | Me | Me—C≡C |
| 78 | H | isothiazol-2-yl |
| 79 | 1-OH-cyclopentyl-C≡C— | H |
| 80 | cyclohexyl-C≡C— | H |
| 81 | cyclohexen-1-yl-C≡C— | H |
| 82 | HOCH₂—C≡C— | H |
| 83 | CH₃CH(OH)C≡C— | H |
| 84 | C₆H₅—C≡C— | H |
| 85 | CH₃C(═O)O | H |

TABLE 14
Prophetic Examples of Compounds

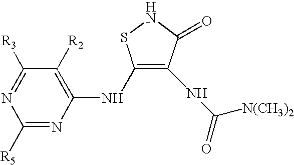

| Compound # | R₂ = | R₃ = | R₅ = |
|---|---|---|---|
| 86 | H | OH | Br |
| 87 | H | OMe | Br |
| 88 | Cl | OH | Cl |
| 89 | Cl | OMe | Cl |
| 90 | H | OMe | OMe |
| 91 | H | H₂N—SO₂— | Br |
| 92 | H | CH₃C(═)NH | H |
| 93 | Cl | Me | H₂N—SO₂— |
| 94 | Cl | OH | Me |
| 95 | Cl | OMe | Me |
| 96 | Me | Me | H₂N—SO₂— |
| 97 | Br | Me | Br |
| 98 | F | CF₃ | F |
| 99 | F | Br | F |
| 100 | Cl | Me | Cl |
| 101 | Cl | CH₃C(═O)O | H |
| 103 | Me | I | Cl |
| 104 | OMe | Cl | OMe |
| 105 | CF₃ | Me | CF₃ |
| 57 | H | HOCH₂—C≡C— | H |
| 58 | H | CH₃CH(OH)C≡C— | CF₃ |
| 59 | H | C₆H₅—C≡C— | CF₃ |
| 60 | F | F | H |

Compounds of Formulas VII, VIII, and IX can be prepared from 5-arylamino-3-alkoxy-isothiazole-4-carbonitriles and conventional reagents, as shown in Schemes 5-7. All procedures are standard. In certain cases, which will be obvious to those skilled in the art, protecting groups will be required. These are typical protecting groups which are well-known in the art of synthetic organic chemistry.

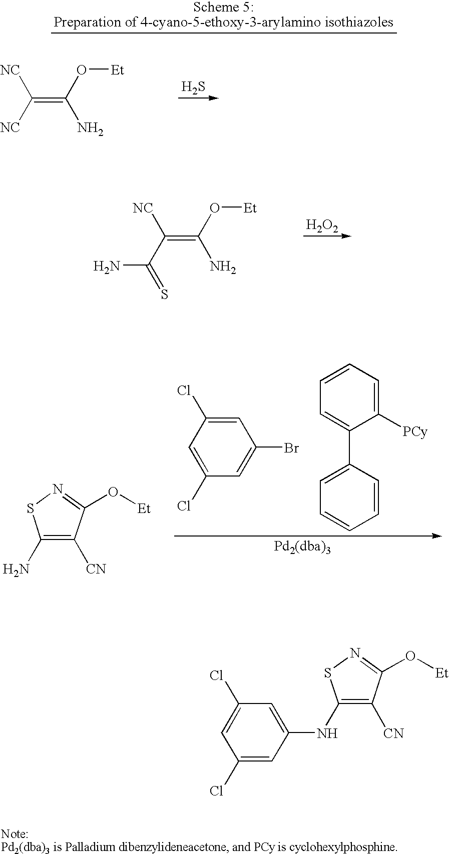

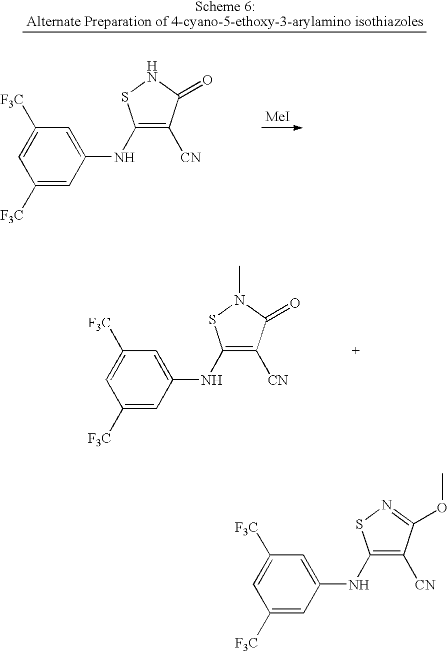

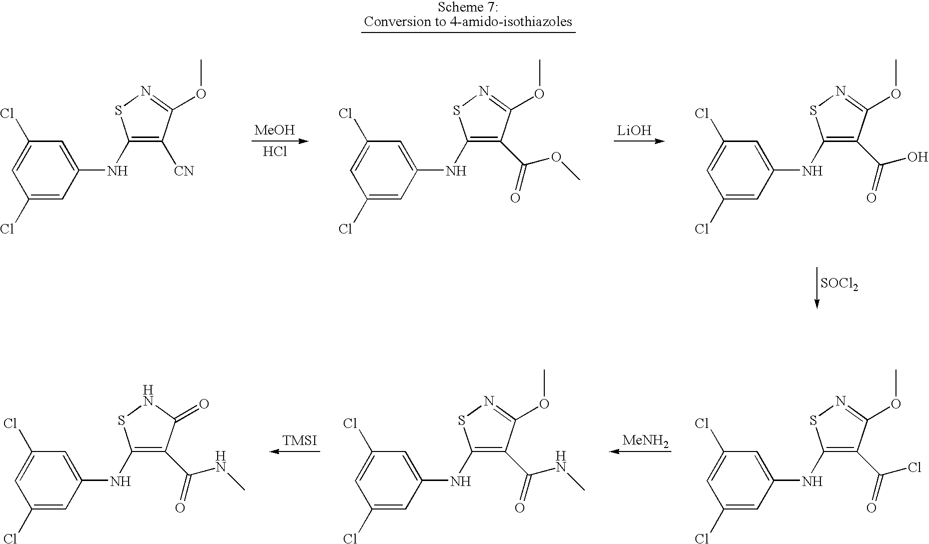

Additional prophetic are shown in tables below.

TABLE 15

| | |
|---|---|
| 1. | 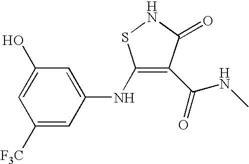 |
| 2. | 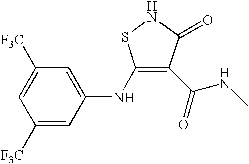 |
| 3. | 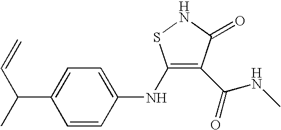 |
| 4. | 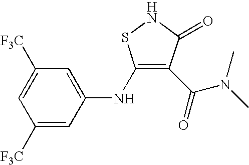 |
| 5. | 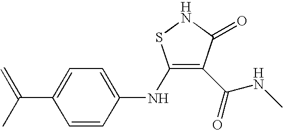 |
| 6. | 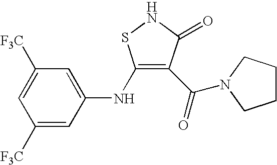 |
| 7. | 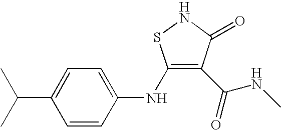 |
| 8. | 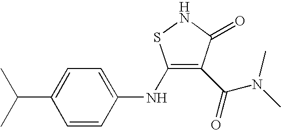 |
| 9. | 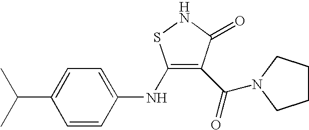 |
| 10. | 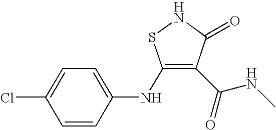 |
| 11. | 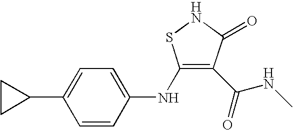 |
| 12. | 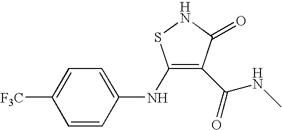 |
| 13. | 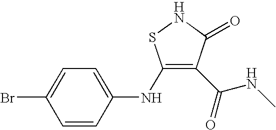 |
| 14. | 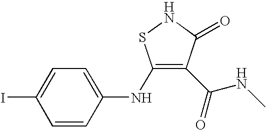 |
| 15. | 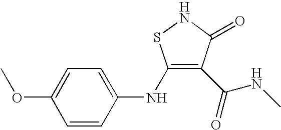 |
| 16. | 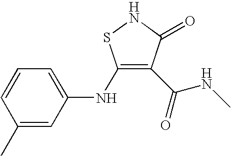 |
| 17. | 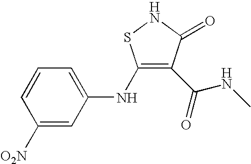 |
| 18. | 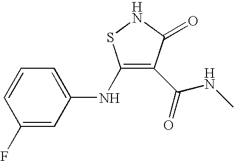 |
| 19. | 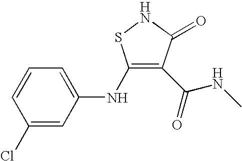 |
| 20. | 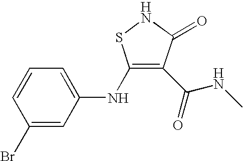 |
| 21. | 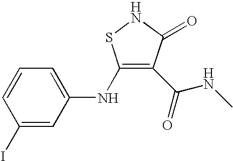 |
| 22. | 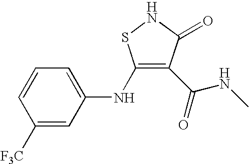 |
| 23. | 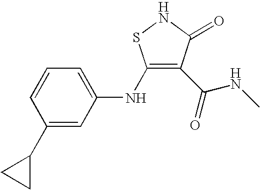 |
| 24. | 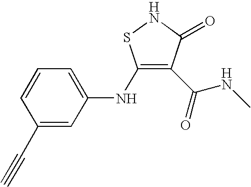 |
| 25. | 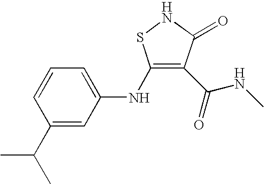 |
| 26. | 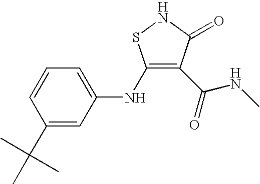 |
| 27. | 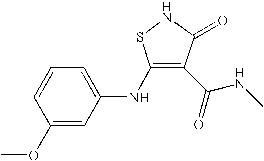 |
| 28. | 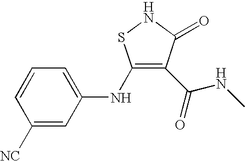 |
| 29. | 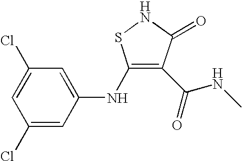 |
| 30. | 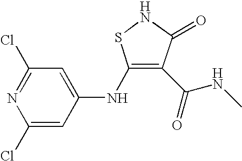 |
| 31. | 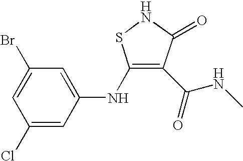 |
| 32. | 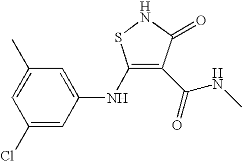 |
| 33. | 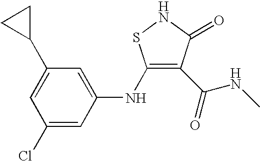 |
| 34. | 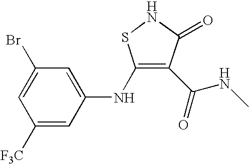 |
| 35. | 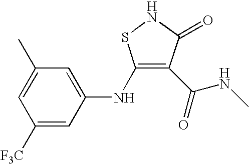 |
| 36. | 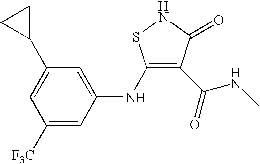 |
| 37. | 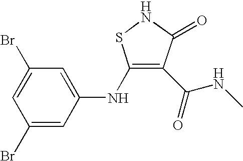 |
| 38. | 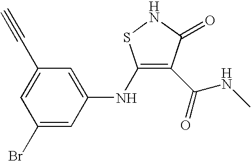 |
| 39. | 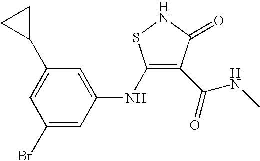 |
| 40. | 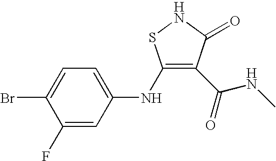 |
| 41. | 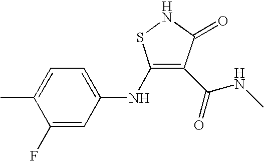 |
| 42. | 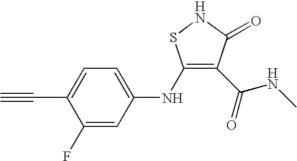 |
| 43. | 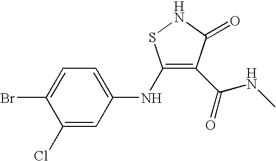 |
| 44. | 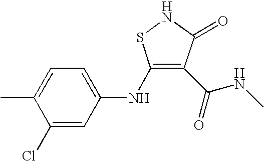 |
| 45. | 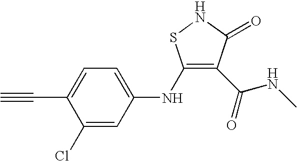 |
| 46. | 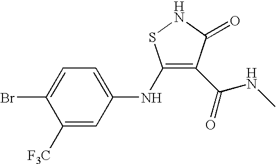 |
| 47. | 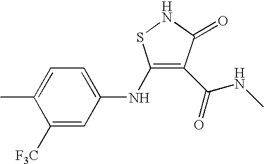 |
| 48. | 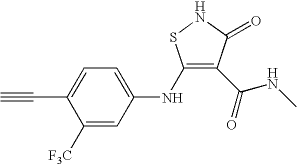 |

TABLE 16

| | |
|---|---|
| 1. | 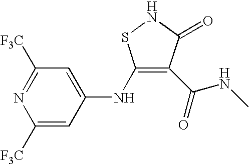 |
| 2. | 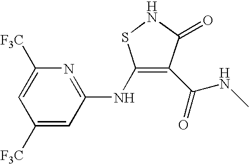 |
| 3. | 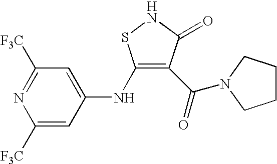 |
| 4. | 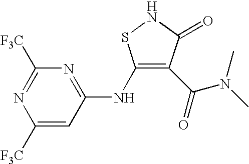 |
| 5. | 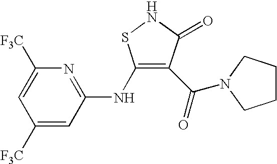 |
| 6. | 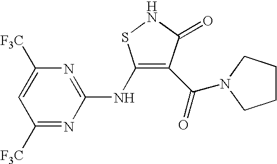 |
| 7. | 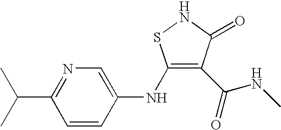 |
| 8. | 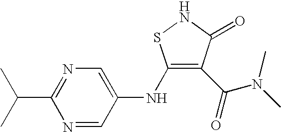 |
| 9. | 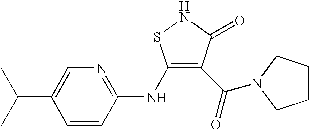 |
| 10. | 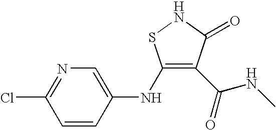 |
| 11. | 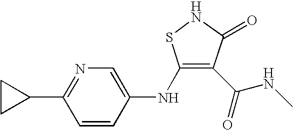 |
| 12. | 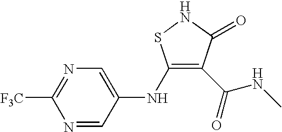 |
| 13. | 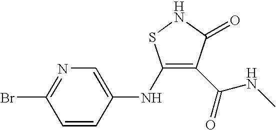 |
| 14. | 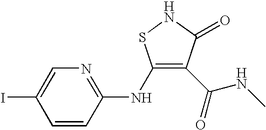 |
| 15. | 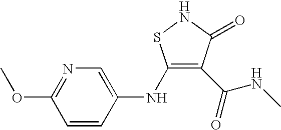 |
| 16. | 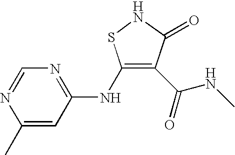 |
| 17. | 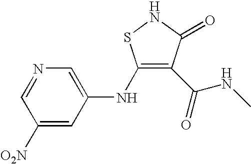 |
| 18. | 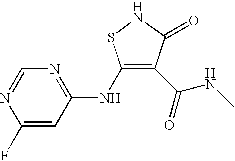 |
| 19. | 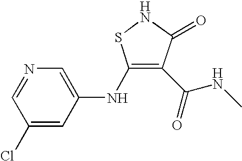 |
| 20. | 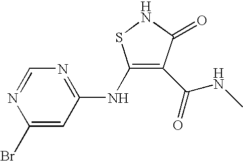 |
| 21. | 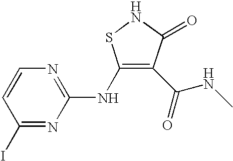 |
| 22. | 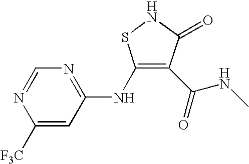 |
| 23. | 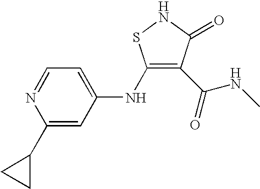 |
| 24. | 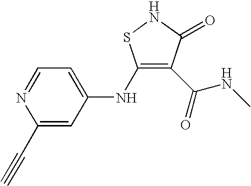 |
| 25. | 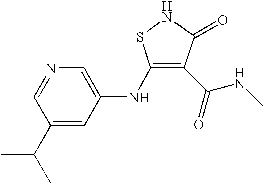 |
| 26. | 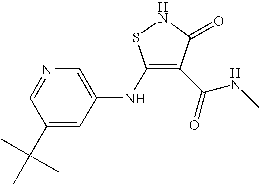 |
| 27. | 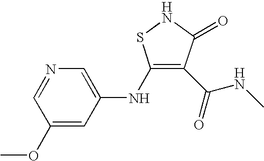 |
| 28. | 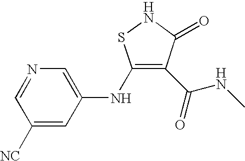 |
| 29. | 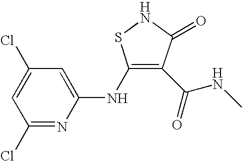 |
| 30. | 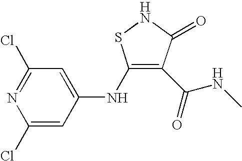 |
| 31. | 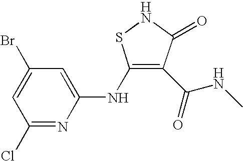 |
| 32. | 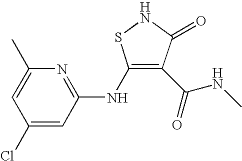 |
| 33. | 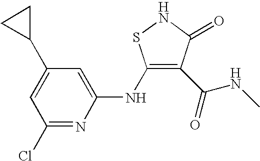 |
| 34. | 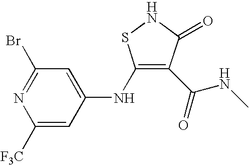 |
| 35. | 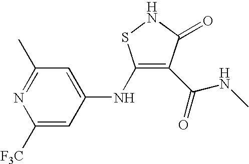 |
| 36. | 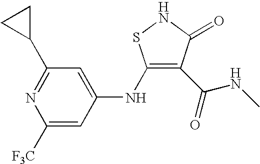 |
| 37. | 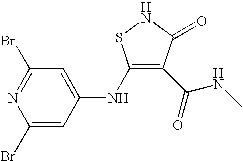 |
| 38. | 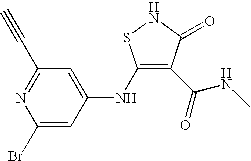 |
| 39. | 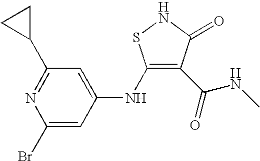 |
| 40. | 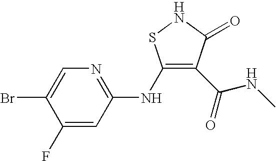 |
| 41. | 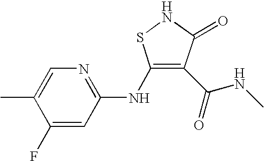 |
| 42. | 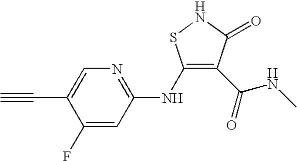 |
| 43. | 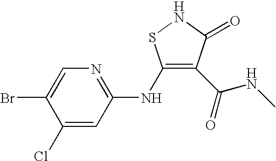 |
| 44. | 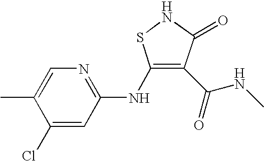 |
| 45. | 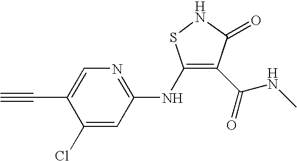 |
| 46. | 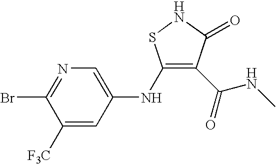 |
| 47. | 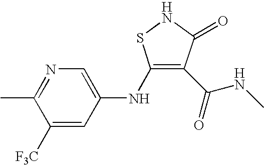 |
| 48. | 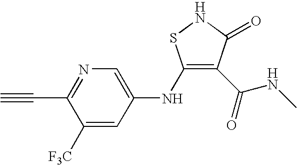 |

Additional contemplated compounds and prophetic examples are shown in the tables below.

TABLE 16
Prophetic Examples of Compounds of the Type

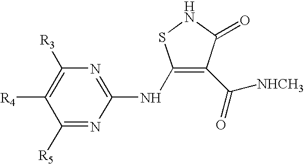

| Com- | | | |
|---|---|---|---|
| pound # | R₃ = | R₄ = | R₅ = |
| 1 | Br | OH | Br |
| 2 | Br | OMe | Br |
| 3 | Cl | OH | Cl |
| 4 | Cl | OMe | Cl |
| 5 | OMe | OMe | OMe |
| 6 | Br | H₂N—SO₂— | Br |
| 7 | Cl | CH₃C(═O)NH | H |
| 8 | Cl | Me | H₂N—SO₂— |
| 9 | Cl | OH | Me |
| 10 | Cl | OMe | Me |
| 11 | Me | Me | H₂N—SO₂— |
| 12 | Br | Me | Br |
| 13 | F | CF₃ | F |
| 14 | F | Br | F |
| 15 | Cl | Me | Cl |
| 16 | Cl | CH₃C(═O)O | H |
| 17 | Me | I | Cl |
| 18 | OMe | Cl | OMe |
| 19 | CF₃ | Me | CF₃ |
| 20 | Cl | H | Cl |
| 21 | Cl | I | H |
| 22 | Me | Me—C≡C | H |
| 23 | isothiazol-3-yl | H | CF₃ |
| 24 | 1-OH-cyclopentyl-C≡C— | H | CF₃ |
| 25 | cyclohexyl-C≡C— | H | CF₃ |
| 26 | cyclohexen-1-yl-C≡C— | H | CF₃ |
| 27 | HOCH₂—C≡C— | H | CF₃ |
| 28 | CH₃CH(OH)C≡C— | H | CF₃ |
| 29 | C₆H₅—C≡C— | H | CF₃ |
| 30 | CH₃C(═O)O | H | CN |

TABLE 17
Prophetic Examples of Compounds of the Type

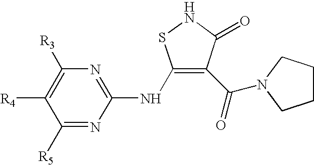

| Com- | | | |
|---|---|---|---|
| pound # | R₃ = | R₄ = | R₅ = |
| 31 | Br | OH | Br |
| 32 | Br | OMe | Br |
| 33 | Cl | OH | Cl |
| 34 | Cl | OMe | Cl |
| 35 | OMe | OMe | OMe |
| 36 | Br | H₂N—SO₂— | Br |
| 37 | Cl | CH₃C(═O)NH | H |
| 38 | Cl | Me | H₂N—SO₂— |
| 39 | Cl | OH | Me |
| 40 | Cl | OMe | Me |
| 41 | Me | Me | H₂N—SO₂— |
| 42 | Br | Me | Br |
| 43 | F | CF₃ | F |
| 44 | F | Br | F |
| 45 | Cl | Me | Cl |
| 46 | Cl | CH₃C(═O)O | H |
| 47 | Me | I | Cl |
| 48 | OMe | Cl | OMe |
| 49 | CF₃ | Me | CF₃ |
| 50 | Cl | H | Cl |
| 51 | Cl | I | H |
| 52 | Me | Me—C≡C | H |
| 53 | isothiazol-3-yl | H | CF₃ |
| 54 | 1-OH-cyclopentyl-C≡C— | H | CF₃ |
| 55 | cyclohexyl-C≡C— | H | CF₃ |
| 56 | cyclohexen-1-yl-C≡C— | H | CF₃ |
| 57 | HOCH₂—C≡C— | H | CF₃ |
| 58 | CH₃CH(OH)C≡C— | H | CF₃ |
| 59 | C₆H₅—C≡C— | H | CF₃ |
| 60 | H | thien-3-yl | F |

TABLE 18
Prophetic Examples of Compounds of the Type

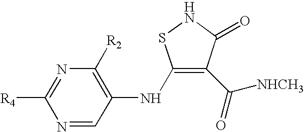

| Compound # | R₂ = | R₄ = |
|---|---|---|
| 61 | H | OH |
| 62 | H | OMe |
| 63 | Me | OH |
| 64 | Cl | OMe |
| 65 | H | OMe |
| 66 | H | H₂N—SO₂— |
| 67 | Cl | CH₃C(═O)NH |
| 68 | Cl | H₂N—SO₂— |
| 69 | Cl | OH |
| 70 | Cl | OMe |
| 71 | Me | Me |
| 72 | F | F |
| 73 | F | CF₃ |
| 74 | F | Br |
| 75 | Cl | Me |
| 76 | H | CH₃C(═O)O |
| 77 | Me | Me—C≡C |
| 78 | H | isothiazol-3-yl |
| 79 | 1-OH-cyclopentyl-C≡C— | H |
| 80 | cyclohexyl-C≡C— | H |
| 81 | cyclohexen-1-yl-C≡C— | H |
| 82 | HOCH₂—C≡C— | H |
| 83 | CH₃CH(OH)C≡C— | H |
| 84 | C₆H₅—C≡C— | H |
| 85 | CH₃C(═O)O | H |

TABLE 19
Prophetic Examples of Compounds of the Type

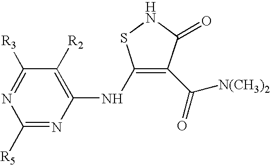

| Compound # | R₂ = | R₃ = | R₅ = |
|---|---|---|---|
| 86 | H | OH | Br |
| 87 | H | OMe | Br |
| 88 | Cl | OH | Cl |
| 89 | Cl | OMe | Cl |
| 90 | H | OMe | OMe |
| 91 | H | H₂N—SO₂— | Br |
| 92 | H | CH₃C(═O)NH | H |
| 93 | Cl | Me | H₂N—SO₂— |
| 94 | Cl | OH | Me |
| 95 | Cl | OMe | Me |
| 96 | Me | Me | H₂N—SO₂— |
| 97 | Br | Me | Br |
| 98 | F | CF₃ | F |
| 99 | F | Br | F |
| 100 | Cl | Me | Cl |
| 101 | Cl | CH₃C(═O)O | H |
| 103 | Me | I | Cl |
| 104 | OMe | Cl | OMe |
| 105 | CF₃ | Me | CF₃ |
| 57 | H | HOCH₂—C≡C— | H |
| 58 | H | CH₃CH(OH)C≡C— | CF₃ |
| 59 | H | C₆H₅—C≡C— | CF₃ |
| 60 | | | |

Biological Studies of Exemplary Compounds
1. HCV Replicon Assay

A human hepatoma cell line (Huh-7) containing replicating HCV Con1 subgenomic replicon with a luciferase reporter gene (luc-ubi-neo) was used to evaluate anti-HCV activity of the compounds. In this assay, the level of luciferase signal correlates directly with the viral RNA replication. The HCV replicon-reporter cell line (NK/luc-ubi-neo) was cultured in DMEM medium supplemented with 10% fetal bovine serum and 0.5 mg/ml Geneticin (G418). Cells were maintained in a subconfluent state to ensure high levels of HCV replicon RNA synthesis.

To evaluate the antiviral activity of compounds, serial dilutions were prepared with concentrations ranging from 0.14 to 300 μM. Diluted compounds were transferred to a 96-well plate followed by the addition of replicon cells (6000 cells per well). Cells were incubated with the compounds for 48 hours after which luciferase activity was measured. Reduction of luciferase signal reflected the decrease of HCV replicon RNA in the treated cells and was used to determine the EC₅₀ value (concentration which yielded a 50% reduction in luciferase activity). A series of compounds with various substitutions were analyzed in the HCV replicon assay and their biological activities were summarized in Table 20.

2. Cytotoxicity Assay

A Huh-7 cell line carrying a luciferase reporter gene (driven by a HIV LTR promoter) stably integrated into the chromosome was used to analyze the cytotoxic effect of the selected compounds. This cell line (LTR-luc) was maintained in DMEM medium with 10% FBS. Design of the cytotoxicity assay was similar to that of the HCV replicon assay. Reduction of luciferase activity in the treated cells correlated with the cytotoxic effect of the test compound and was used to calculate the CC₅₀ value (concentration that inhibited cell growth by 50%). As shown in Table 4, most of the compounds were not toxic to Huh-7 cells at concentrations up to 50 μM, indicating that compounds have direct inhibitory effect on viral RNA replication in the HCV replicon cells.

3. In vitro HCV Replicase Complex Assay

Crude membrane fraction containing the replicase complexes (RC) was isolated from the HCV subgenomic replicon cell line (I₃₈₉/NS3-3) (Lohmann et al., Science 285:110-113, 1999) through Dounce homogenization and high-speed centrifugation (Lai et al., J. Virol. 73:2295-2300). Standard assay mixtures contained 50 mM HEPES (pH 7.3), 10 mM KCl, 10 mM MgCl₂, 20 units of ribonuclease inhibitor, 10 μg/ml actinomycin D, 0.5 mM of ATP, GTP, CTP, 10 μCi of [α-³³P] UTP and 0.5 μl of the membrane fraction in a total volume of 20 μl. To determine the inhibitory effect of compounds on the HCV replicase complexes, pre-incubation of the RC membrane fraction with tested compounds was performed in a reaction mixture at 4° C. for 15 minutes. The reaction was then initiated by the addition of the radiolabeled UTP and cold nucleotides and incubated at 30° C. for 1 hour. Products were extracted with phenol/chloroform, precipitated with ethanol, and separated on a 1% agarose gel. After electrophoresis, the gel was dried prior to autoradiography. Radioactivity incorporated into viral RNA was quantitated by using a Phosphorimager (Amersham Biosciences Corp., Piscataway, N.J.) and was used to determine the IC₅₀ value. The activities of selected compounds in the replicase complex assay were summarized in Table 2. A number of the exemplary compounds were potent inhibitors that blocked viral RNA synthesis catalyzed by HCV replicase complexes, with IC₅₀ values in the nM range (Table 2).

4. Isolation of Resistant Replicon

A replicon-containing cell line (I₃₈₉/NS3-3) was used to select the resistant replicons against Compound 1 (Lohmann et al., Science 285:110-113, 1999). Replicon cells were plated in a 100 mm dish, under a subconfluent condition (approximately at 40% confluency). After 24 hours, Compound 1 was added to the cells with the final concentration of 2 μM in the medium containing 1 mg of G418 per ml. After 3 weeks, colonies of cells resistant to Compound 1 were isolated and expanded. Three independent cell lines (w1, w3 and w4) with growth rate similar to that of the parental replicon cells were selected to test for their sensitivity to Compound 1.

To confirm that viral RNAs in the selected cell lines were resistant to Compound 1, the cells were treated with the compound with concentrations from 0.05 to 5 μM. After 72-hours of treatment, cells were lysed and the cell lysates were diluted with RNase-free water, from which 5 μl was used to quantify the HCV replicon RNA by using real-time quantitative reverse transcriptase (RT) PCR assay (Cheney et al., Virology 297:298-306, 2002). Same quantitative RT-PCR protocol was used to determine the level of the cellular glyceraldhehyde-3-phosphate dehydrogenase (GAPDH) RNA in the same cell lysate, which served as an internal control to normalize the difference of total RNAs. To calculate the EC₅₀ values of Compound 1 against the resistant cell lines, the relative cycle threshold (CT) for the HCV and GAPDH was determined. Replication of HCV RNA in the three selected cell lines was more resistant to Compound 1, with 29 to 66 folds increase in the EC50 values when compared with that of the parental replicon cells (Table 3). These cell lines were still sensitive to inhibition by the nucleoside inhibitor 2′-C-methyl-adenosine (Carroll et al., J. Biol. Chem 278:11979-11984, 2003), suggesting that resistance was specific to Compound 1 (Table 3). 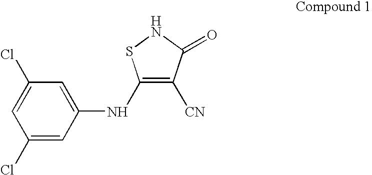
5. Identification of the Molecular Target

To determine the targeted viral gene of Compound 1, total RNA was isolated from the resistant cell lines and from the parental replicon cells. The cDNA fragment encoding the HCV nonstructural proteins (NS3-NS5B) was amplified from the total cellular RNA by using a Thermoscript RT-PCR kit (Invitrogen). The cDNA fragments were cloned into pCR4-TOPO vector by using a TOPO TA cloning kit (Invitrogen). The entire DNA sequence of the HCV nonstructural coding region was determined for multiple independent cDNA clones.

Eight different cDNA clones were generated from three independently cell lines selected by Compound 1 (cell lines w1, w3 and w4) and three cDNA clones from the parental cell line. DNA sequence analysis revealed that a single nucleotide change in the region of viral NS5B polymerase was present in all the eight cDNA clones of the three resistant cell lines. This mutation resulted in a change of the amino acid residue cysteine at position 316 of NS5B polymerase to a tyrosine residue (amino acid 2735 in the full-length of the HCV polyprotein).

The biological activities and cytotoxicity of the selected compounds are summarized in Table 20.

TABLE 20
Activity against HCV replicon and cytotoxicity of selected compounds

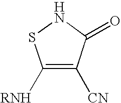

| | Range | |
|---|---|---|
| | of EC₅₀ | |
| | in HCV | Range of CC₅₀ |
| | replicon | in cytotoxicity |
| R | assay | assay |
| 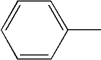 | B | B |
| 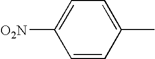 | D | B |
| 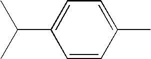 | B | A |
| 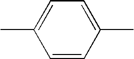 | D | B |
| 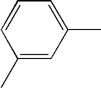 | B | B |
| 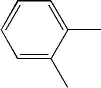 | C | B |
| 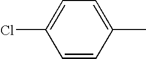 | B | B |
| 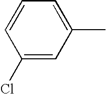 | B | B |
| 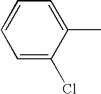 | C | B |
| 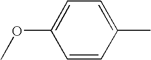 | C | B |
| 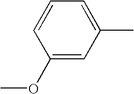 | D | B |
| 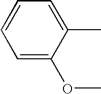 | C | B |
| 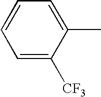 | C | B |
| 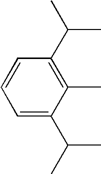 | C | B |
| 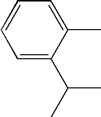 | D | B |
| 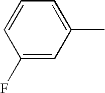 | B | B |
| 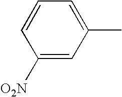 | B | B |
| 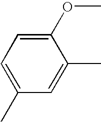 | C | B |
| 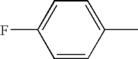 | C | B |
| 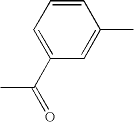 | D | B |
| 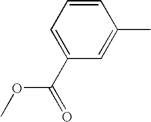 | D | B |
| 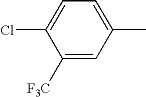 | B | B |
| 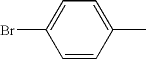 | A | B |
| 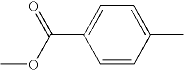 | C | B |
| 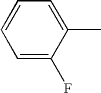 | C | B |
| 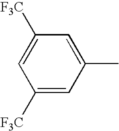 | B | B |
| 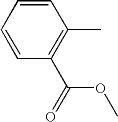 | D | B |
| 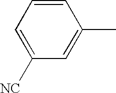 | B | B |
| 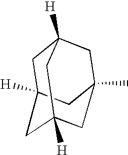 | B | B |
| 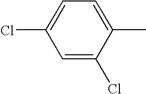 | C | B |
| 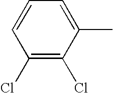 | D | B |
| 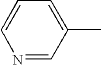 | C | B |
| 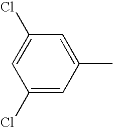 | A | B |
| 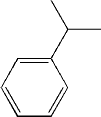 | C | B |
| 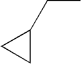 | D | B |
| 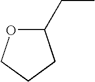 | D | B |
| 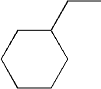 | B | B |
| 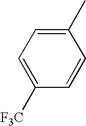 | B | B |
| 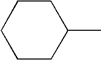 | D | B |
| 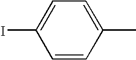 | B | B |
| 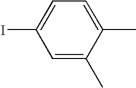 | C | B |
| 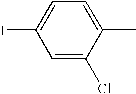 | B | A |
| 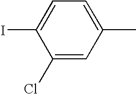 | A | B |
| 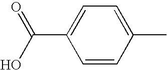 | D | B |
| 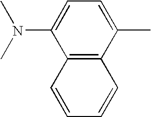 | D | B |
| 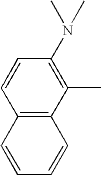 | D | B |
| 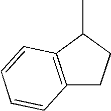 | B | B |
| 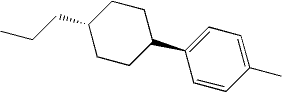 | B | A |
| 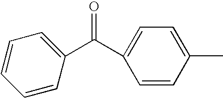 | C | B |
| 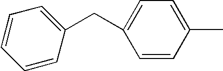 | D | B |
| 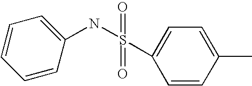 | D | B |
| 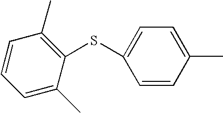 | B | A |
| 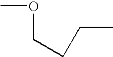 | D | B |
| 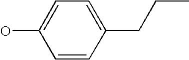 | D | B |
| 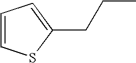 | D | B |
| 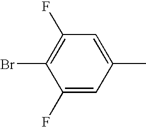 | A | B |
| 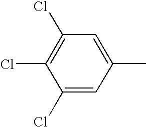 | A | A |
| 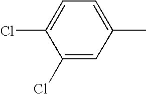 | A | B |
| 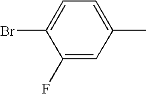 | A | B |
| 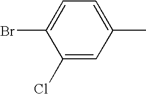 | A | B |
| 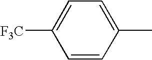 | B | B |
| 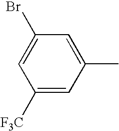 | A | B |
| 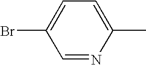 | C | B |
| 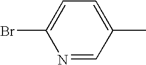 | C | B |
| 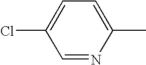 | C | B |
| 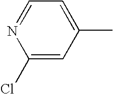 | C | B |
| 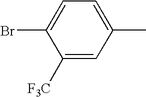 | B | B |
| 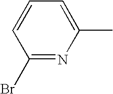 | C | B |
| 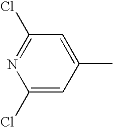 | B | A |
| 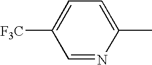 | C | B |

Anti-HCV activities (EC₅₀ values) of the test compounds were categorized into four different groups: A (<1 μM), B (1 to 10 μM), C (10 to 50 μM), and D (>50 μM).
The cytotoxicity (CC₅₀ values) of the compounds was categorized into two groups: A (<50 μM) and B (>50 μM).

TABLE 21
Activity of selected compounds in the HCV replicase complex assay

| Compound | IC₅₀ (nM) |
|---|---|
| 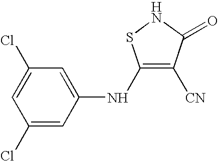 | A |
| 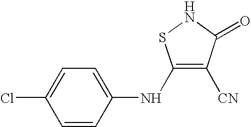 | B |
| 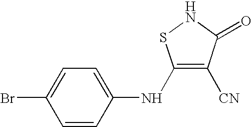 | A |
| 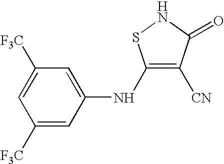 | C |
| 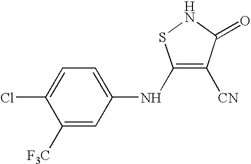 | B |

A = 0-50 nM;
B = 50-100 nm;
C = 100-250 nM

TABLE 22
Sensitivity of wild-type and resistant
replicon cell lines to Compound 1

| | |
|---|---|
| Compound 1 | 2′-C-methyl-adenosine |

| | | Fold over | | Fold over |
|---|---|---|---|---|
| Cell line | EC₅₀ (μM) | wild-type | EC₅₀ (μM) | wild-type |
| Wild-type | 0.07 | 1 | 0.36 | 1 |
| W1 | 3.68 | 52 | 0.16 | 0.4 |
| W3 | 2.03 | 29 | 0.26 | 0.7 |
| W4 | 4.62 | 66 | 0.31 | 0.9 |

What is claimed is

1. A compound of Formula I or a salt thereof 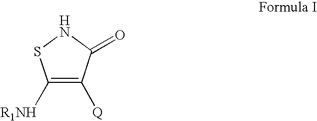
where Q is CN, NHCONRₐR_{b}, or CONRₐR_{b}, where Rₐ is C₁-C₃ alkyl or C₁-C₃ alkenyl, said alkyl and said alkenyl groups optionally substituted with, independently, 1, 2, or 3 halogen atoms, O—C₁₋₂alkyl, C₁-C₃ alkenyl, and N(H)C₁₋₂ alkyl, R_{b} is H or C₁-C₃ alkyl; or Rₐ and R_{b}, together with the atoms to which they are attached, form a 5- or 6-membered ring, optionally containing one or two ring double bonds, and optionally containing one ring oxygen atom or one additional ring nitrogen atom; and R₁ is cyclohexyl, adamantan-1-yl, indan-1-yl, Ar¹(CH₂)ₙ, or Ar², where n is zero or 1, Ar¹ is 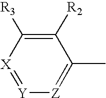
where X is C—R₄ or N, Y is C—R₅ or N, and Z is CH or N, provided that Ar contains no more than two ring nitrogen atoms; R₂, R₃, R₄, and R₅ are, independently, H, F, Cl, Br, I, OH, CN, CF₃, NO₂, C₁-C₆ alkyl, C₂-C₆ alkenyl, C₃-C₆ cycloalkyl, C₅-C₆ cycloalkenyl, O—C₁-C₆ alkyl, O—C₂-C₆ alkenyl, C₁-C₆ alkyl-C(═O)—, C₁-C₆ alkyl-O—C(═O)—, C₁-C₆ alkenyl-O—C(═O)—, C₁-C₆ alkyl-C(═O)O—, C₁-C₆ alkenyl-C(═O)O, isothiazolyl, isoxazolyl, isoxazolinyl, isoxazolidinyl, oxazolyl, oxazolinyl, oxazolidinyl, thiazolyl, thienyl, furyl, pyrrolyl, pyrrolinyl, pyrrolidinyl, thiazolinyl, thiazolidinyl, isothiazolinyl, isothiazolidinyl, imidazolinyl, imidazolyl, pyridyl, piperidinyl, phenyl, CH₃SO₂—, NH₂SO₂—, CH₃NHSO₂—, CH₃SO₂NH—, R₆R₇N—, R₆R₇NCH₂—, R₇C(═O)NH, or R₆R₇NC(═O), wherein R₆ and R₇ are, independently, H, C₁-C₄ alkyl, C₂-C₆ alkenyl; R₈—C≡C—, wherein R₈ is H, C₁-C₆ alkyl, C₂-C₆ alkenyl, C₃-C₆ cycloalkyl, C₅-C₆ cycloalkenyl, (CH₃)₂NCH₂—, (CH₃)₂NCH₂CH₂—, or phenyl; wherein all alkyl, cycloalkyl, alkenyl, and cycloalkenyl groups in Rₐ-R_{b} and R₂-R₈ are optionally substituted with one, two, or three halogen atoms, one C₁-C₃ alkyl group, or one hydroxyl group; and all aryl and heteroaryl groups in Rₐ-R_{b} and R₂-R₈ are optionally substituted with one hydroxy group and with one, two, or three groups selected from F, Cl, Br, I, and C₁-C₄ alkyl, provided that when n is zero and Ar is phenyl, then 1) R₂ is either H or halogen; and 2) at least one of R₂, R₃, R₄, and R₅ is neither H not C; and further provided that Ar is not 4-chloro-3-trichloromethyl-phenyl;
and Ar² is 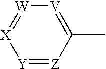
wherein V is N or CR₂, W is N or CR₃, X is N or CR₄, Y is N or CR₅, and Z is N or CH, provided that exactly two of V, W, X, Y, and Z are N, and further provided that the two ring nitrogen atoms are not adjacent, and R₂-R₅ are, independently, H, F, Cl, Br, I, OH, CN, CF₃, NO₂, C₁-C₆ alkyl, C₂-C₆ alkenyl, C₃-C₆ cycloalkyl, C₅-C₆ cycloalkenyl, C₅-C₆ cycloalkadienyl, O—C₁-C₆ alkyl, O—C₂-C₆ alkenyl, C₁-C₆ alkyl-C(═O)—, C₁-C₆ alkyl-O—C(═O)—, C₁-C₆ alkenyl-O—C(═O)—, C₁-C₆ alkyl-C(═O)O—, C₁-C₆ alkenyl-C(═O)O, isothiazolyl, isoxazolyl, isoxazolinyl, isoxazolidinyl, oxazolyl, oxazolinyl, oxazolidinyl, thiazolyl, thienyl, furyl, pyrrolyl, pyrrolinyl, pyrrolidinyl, thiazolinyl, thiazolidinyl, isothiazolinyl, isothiazolidinyl, imidazolinyl, imidazolyl, pyridyl, piperidinyl, phenyl, CH₃SO₂—, NH₂SO₂—, CH₃NHSO₂—, CH₃SO₂NH—, R₆R₇N—, R₆R₇NCH₂—, R₇C(═O)NH, or R₆R₇NC(═O), wherein R₆ and R₇ are, independently, H, C₁-C₄ alkyl, C₂-C₆ alkenyl; R₈—C≡C—, wherein R₈ is H, C₁-C₆ alkyl, C₂-C₆ alkenyl, C₃-C₆ cycloalkyl, C₅-C₆ cycloalkenyl, (CH₃)₂NCH₂—, (CH₃)₂NCH₂CH₂—, or phenyl; wherein all alkyl, cycloalkyl, alkenyl, and cycloalkenyl groups in R₂-R₅ and R₆-R₈ are optionally substituted with one, two, or three halogen atoms, one C₁-C₃ alkyl group, or one hydroxyl group; and all aryl and heteroaryl groups in R₂-R₅ and R₆-R₈ are optionally substituted with one, two, or three groups selected from F, Cl, Br, I, and C₁-C₄ alkyl;
Rₐ is H, C₁-C₃ alkyl or C₁-C₃ alkenyl, optionally substituted with O—C₁₋₂ alkyl, C₁-C₃ alkenyl, or N(H)C₁₋₂ alkyl; and R_{b} is C₁-C₃ alkyl, or Rₐ and R_{b}, together with the atoms to which they are attached, form a 5- or 6-membered ring, optionally containing one or two ring double bonds, and optionally containing one ring oxygen atom or one additional ring nitrogen atom.

2. The compound of claim 1, or a salt thereof, where R₁ is Ar¹(CH₂)ₙ.

3. The compound of claim 2, where Q is CN.

4. The compound of claim 2, where Q is NHCONRₐR_{b}.

5. The compound of claim 2, where Q is CONRₐR_{b}.

6. The compound of any of claims 3, 4, and 5, where X is C—R₄, Y is C—R₅, and Z is CH.

7. The compound of claim 6, where n is zero and NRₐR_{b} is pyrrolidino, NEt₂, NHCH₃ or N(CH₃)₂.

8. The compound of claim 6, where n is 1 and NRₐR_{b} is pyrrolidino, NHCH₃ or N(CH₃)₂.

9. The compound of claim 7, where none of R₃, R₄, or R₅ is H.

10. The compound of claim 8, where none of R₃, R₄, or R₅ is H.

11. The compound of claim 9 or claim 10, where at least one of R₃, R₄, or R₅ is selected from the group consisting of halogen, C₂-C₄ alkenyl, C₁-C₄ alkyl, C₁-C₄ alkoxy, C₂-C₄ alkynyl, C₃-C₆ cycloalkyl, acetyl, and trifluoromethyl.

12. The compound of claim 7 or claim 8, where either one or both of R₃ and R₅ are selected from the group consisting of halogen, methoxy, methyl, cyclopropyl, and trifluoromethyl.

13. The compound of any of claims 3, 4, and 5, where X is N, Y is C—R₅, and Z is CH.

14. The compound of any of claims 3, 4, and 5, wherein X is C—R₄, Y is N, and Z is CH.

15. The compound of any of claims 3, 4, and 5, wherein X is C—R₄, Y is C—R₅, and Z is N.

16. The compound of any of claims 3, 4, and 5, where n is zero.

17. The compound of claim 13, where R₃ is selected from the group consisting of halogen, C₂-C₄ alkenyl, C₁-C₄ alkyl, C₁-C₄ alkoxy, C₂-C₄ alkynyl, C₃-C₆ cycloalkyl, cyano, acetyl, and trifluoromethyl.

18. The compound of claim 17, where R₅ is H, cyano, C₂-C₄ alkenyl, C₁-C₄ alkyl, C₁-C₄ alkoxy, C₂-C₄ alkynyl, C₃-C₆ cycloalkyl, (C₁-C₄ alkyl)-C(═O)—, (C₁-C₄ alkyl)-C(═O)O—, (C₁-C₄ alkyl)-O—C(═O)—, (C₁-C₃ alkyl)-C(═O)NH—, (C₁-C₃ alkyl)NH—, methyl sulfonyl, methylsulfonylamino, and trifluoromethyl.

19. The compound of claim 14, where R₃ is selected from the group consisting of halogen, C₂-C₄ alkenyl, C₁-C₄ alkyl, C₁-C₄ alkoxy, C₂-C₄ alkynyl, C₃-C₆ cycloalkyl, cyano, acetyl, and trifluoromethyl.

20. The compound of claim 19, where R₄ is H, cyano, C₂-C₄ alkenyl, C₁-C₄ alkyl, C₁-C₄ alkoxy, C₂-C₄ alkynyl, C₃-C₆ cycloalkyl, (C₁-C₄ alkyl)-C(═O)—, (C₁-C₄ alkyl)-C(═O)O—, (C₁-C₄ alkyl)-O—C(═O)—, (C₁-C₃ alkyl)-C(═O)NH—, (C₁-C₃ alkyl)NH—, methyl sulfonyl, methylsulfonylamino, and trifluoromethyl.

21. The compound of claim 15, where R₃ is selected from the group consisting of halogen, C₂-C₄ alkenyl, C₁-C₄ alkyl, C₁-C₄ alkoxy, C₂-C₄ alkynyl, C₃-C₆ cycloalkyl, cyano, acetyl, and trifluoromethyl.

22. The compound of claim 21, where R₄ is H, cyano, C₂-C₄ alkenyl, C₁-C₄ alkyl, C₁-C₄ alkoxy, C₂-C₄ alkynyl, C₃-C₆ cycloalkyl, (C₁-C₄ alkyl)-C(═O)—, (C₁-C₄ alkyl)-C(═O)O—, (C₁-C₄ alkyl)-O—C(═O)—, (C₁-C₃ alkyl)-C(═O)NH—, (C₁-C₃ alkyl)NH—, methyl sulfonyl, methylsulfonylamino, and trifluoromethyl.

23. The compound of claim 1, where R¹ is Ar².

24. The compound of claim 23, where Q is CN.

25. The compound of claim 23, where Q is NHCONRₐR_{b}.

26. The compound of claim 23, where Q is CONRₐR_{b}.

27. A compound of Formula IX or a salt thereof, 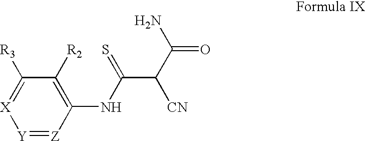
wherein X is N or CR₄, Y is N or CR₅, and Z is N or CH, provided that no more than one of X, Y, and Z is N or CH, where all substituents are as described for Formula I, and provided that at least one of R₂-R₅ is other than H.

28. The compound of claim 23, where is X is CR₄, Y is CR₅, and Z is CH.

29. A method of treating Hepatitis C infection in a human comprising providing to a person in need of treatment thereof a therapeutically effective concentration of a compound of Formula I.

30. The method of claim 29, where the compound is selected from Formulas II, III, IV, V, VI, VII, or VIII.
